(19) United States
(12) Patent Application Publication
(10) Pub. No.: US 2026/0176236 A1
(43) Pub. Date: Jun. 25, 2026

(54) LINEAR AMINO ACID SURFACTANTS HAVING SURFACE-ACTIVE PROPERTIES

(71) Applicant: AdvanSix Resins & Chemicals LLC, Parsippany, NJ (US)

(72) Inventors: Edward Asirvatham, Chatham, NJ (US); Kavssery Parameswaran Ananthapadmanabhan, Loveland, OH (US); Harshita Kumari, Cincinnati, OH (US); Ramana Reddy Mittapalli, Cincinnati, OH (US); Suchitra Dilipkumar Panigrahi, Cincinnati, OH (US)

(21) Appl. No.: 19/429,331

(22) Filed: Dec. 22, 2025

Related U.S. Application Data
(60) Provisional application No. 63/738,280, filed on Dec. 23, 2024.

(51) Int. Cl. C07D 213/04; C07C 229/10; C07C 229/12; C07C 229/14; C07C 229/16; C07C 233/47; C07C 235/12; C07C 237/06; C07C 237/22; C07C 309/14; C07D 295/15; C07D 295/24

(52) U.S. Cl. CPC C07D 213/04; C07C 229/10; C07C 229/12; C07C 229/14; C07C 229/16; C07C 233/47; C07C 235/12; C07C 237/06; C07C 237/22; C07C 309/14; C07D 295/15; C07D 295/24

(57) ABSTRACT

Derivatives of amino acids that have surface-active properties are provided. The amino acid can be naturally occurring or synthetic, or they may be obtained via a ring-opening reaction of a lactam, such as caprolactam. The amino acid can be naturally occurring or synthetic, or they may be obtained via a ring-opening reaction of a lactam, such as caprolactam. The amino acid may be functionalized to form a compound that is surface-active and have advantageous surfactant characteristics. The compounds of the present disclosure have low critical micelle concentrations (CMC) as well as superior ability to lower the surface tension at an interface of otherwise immiscible phases of materials.

### CROSS REFERENCE TO RELATED APPLICATIONS

This application claims priority to U.S. Provisional Patent Application Ser. No. 63/738,280, filed Dec. 23, 2024, the entire disclosure of which is incorporated by reference in its entirety.

### FIELD

The present disclosure pertains to derivatives of amino acids and methods for their synthesis, wherein the derivatives have surface-active properties. The present disclosure therefore provides the derivates of amino acids as surfactants.

### BACKGROUND

Surfactants (molecules with surface-active properties) are an important class of molecules with highly sought-after characteristics. Surfactants may be uncharged, zwitterionic, cationic, or anionic. Often, these compounds are amphiphilic molecules with a water-insoluble hydrophobic “tail” group and a water-soluble hydrophilic “head” group. These compounds may adsorb at an interface, such as an interface between two liquids, a liquid and a gas, or a liquid and a solid. In the case of an interface between water and oil, the hydrophilic head group extends into the water, while the hydrophobic tail extends into the oil. When added to water, the hydrophilic head group extends into the water, while the hydrophobic tail extends into the air. The presence of the surfactant disrupts the intermolecular interaction between water molecules, replacing it with weaker interactions between water molecules and the surfactant. This results in lowered surface tension and can also serve to stabilize the interface.

At sufficiently high concentrations, surfactants may form aggregates to limit the exposure of the hydrophobic tail to the polar solvent. One such aggregate is a micelle, in which the molecules are arranged in a sphere with the hydrophobic tails inside the sphere and the hydrophilic heads on the outside to interact with a polar solvent. The effect that a given compound has on surface tension and the concentration at which it forms micelles may serve as defining characteristics for a surfactant.

Surfactants are used in a wide range of commercial applications in formulations ranging from detergents, agricultural products such as herbicidal and other biocidal formulations, surface cleaning agents used in households and industrial and healthcare settings, paints and coating formulations, personal care products ranging from personal hygiene products to hair care products to cosmetics and the like. In personal care space alone, compounds with surface-active properties are used as soaps, detergents, lubricants, wetting agents, foaming agents, and spreading agents, among others. Surfactants are used in many industrial applications, such as in the micro-electronics/semiconductor chips manufacturing processes, in oil and gas extraction, and in the manufacturing of pharmaceuticals and medicaments, drug delivery vehicles as well as in healthcare facilities such as clinics, physician's office, and hospitals. Thus, there is an ongoing need to identify new surface-active compounds, synthesize, evaluate, and optimize them to make compositions that confer superior surface actions and functionalities in end uses.

However, solely from its structure, it can be difficult to predict whether a given compound would have surface-active properties or the requisite surface-active properties demanded by the end-use, let alone other important characteristics such as interfacial adsorption dynamics, minimum surface tension achievable, and/or ability to wet hydrophobic and/or oleophobic surfaces, which are also integral to whether the compound would become a useful surfactant. Certain amino acids and their derivatives, for example, are desirable as building blocks for surfactants, but the selection of which amino acids to use is far from intuitive. Synthesis of such compounds adds another layer of difficulty due to the differences of solubilities attributable to different elements and moieties present in the same molecules. There remains a need for high-efficacy surfactants that can be readily synthesized at commercial scale via straightforward routes.

### SUMMARY

The present disclosure provides derivatives of amino acids that can be used as surfactants, as they have surface-active properties. The amino acids may be naturally occurring or synthetic amino acids, or they may be obtained via ring-opening reactions of molecules such as lactams, for example caprolactam. The amino acids may be functionalized with different types of siloxane groups to form compounds with surface-active properties. Characteristically, these compounds may have low critical micelle concentrations (CMC) and/or the ability to reduce the surface tension of a liquid.

The present disclosure provides a compound of Formula I, wherein the compound of Formula I can be a salt, below:

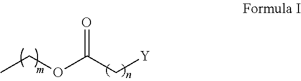

wherein Y is selected from formula (1-1), (1-2), or (1-3)

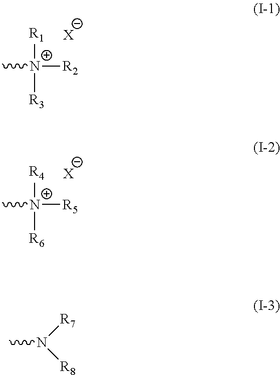

the wave-line denoting the connection to Formula I;
   wherein
R₁ and R₂ form a ring that may be substituted or unsubstituted, saturated or unsaturated; optionally the ring may include one or more of oxygen, nitrogen, or sulfur atoms or groups that include at least one of these atoms at any suitable position in the ring, and the ring may be optionally substituted with one or more substituents selected from the group consisting of alkyl, hydroxyl, amino, amido, sulfonyl, sulfonate, carbonyl, carboxyl, and carboxylate;
   wherein the ring formed from R₁ and R₂ is optionally linked and/or fused to a further ring;
R₃ is selected from the group consisting of hydrogen, alkyl, alkenyl, alkynyl, ester, hydroxyl, aryl alkyl, alkoxy alkyl ether, alkyl phosphate, C₃-C₈ carboxylic acid, C₁-C₁₀ alkyl benzoic acid, oxygen, and sulfur;
R₄ and R₅ may be the same or different, and are selected from the group consisting of hydrogen and C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate;
R₆ is selected from the group consisting of alkenyl, alkynyl, ester, hydroxyl, aryl alkyl, alkoxy alkyl ether, alkyl phosphate, C₃-C₈ carboxylic acid, C₁-C₁₀ alkyl benzoic acid, amino, amido, and sulfur; wherein R⁶ may optionally be substituted with one or more substituents selected from the group consisting of aryl, heteroaryl, hydroxyl, amino, amido, sulfonyl, sulfonate, carbonyl, carboxyl, and carboxylate;
R₇ and R₈ may be the same or different, and are selected from the group consisting of hydrogen and C₁-C₆ alkyl, wherein the C₁-C₆ alkyl is substituted with one or more substituents selected from the group consisting of hydroxyl, amino, amido, sulfonyl, sulfonate, carbonyl, carboxyl, and carboxylate, or a salt thereof, wherein a counterion W associated with any of the hydroxyl, sulfonate, or carboxylate group is selected from Na, K or Ca;
X is selected from the group consisting of chloride, bromide, and iodide.
n is an integer in the range of 2 to 5; and
m is an integer in the range of 9 to 20.

The compound provided by the present disclosure is a compound of Formula I, wherein in R₁ and R₂ form a ring with 4 to 20, with 4 to 15, with 4 to 10, with 4 to 8, with 4, 5 and 6 atoms, or in particular with 6 atoms.

For example, the structure of the formula (1-1) may include piperidine, pyridine, pyrimidine, pyrrole, imidazole, pyrazole, piperazine, or morpholine.

For example, structure of the formula (1-1) is selected from:

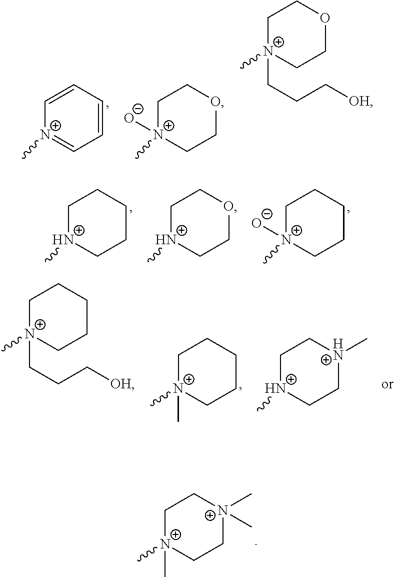

For example, structure of the formula (1-2) or (1-3) is selected from:

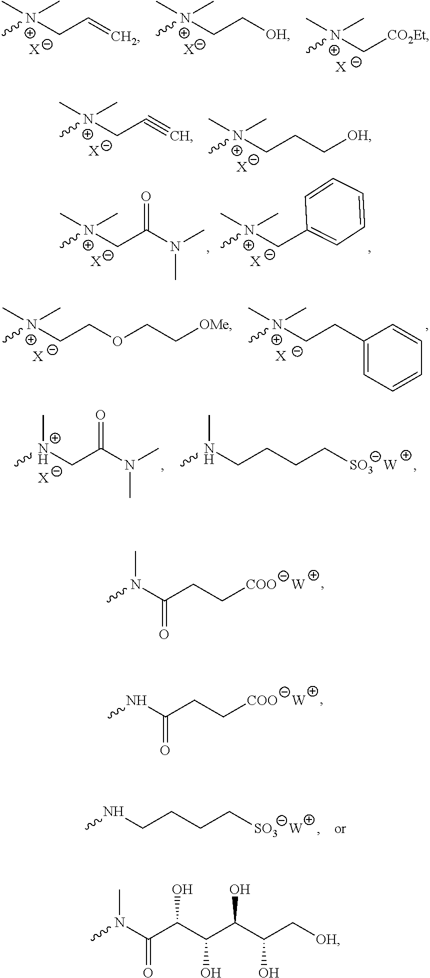

wherein
X is selected from the group consisting of chloride, bromide, and iodide, and W is selected from the group consisting of sodium, potassium, or calcium.

Compounds provided by the present disclosure are compounds of Formula I, wherein n is 2, 3, 4 or 5, preferably 5.

For any of the preceding n values, which can be 2, 3, 4, or 5, the compounds provided by the present disclosure are compounds of Formula I, wherein m is 9, 10, 11, 12, 13, 14, 15, 16, 17, 18, 19, or 20, preferably 11.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a morpholine structure.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a morpholine structure, R₃ is methyl, and X is chloride.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a morpholine structure, R₃ is methyl, and X is iodide.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a morpholine structure, R₃ is methyl, and X is bromide.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a piperidine structure.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a piperidine structure, R₃ is methyl, and X is chloride.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a piperidine structure, R₃ is methyl, and X is iodide.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a piperidine structure, R₃ is methyl, and X is bromide.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a piperazine structure.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a piperazine structure, R₃ is methyl, and X is chloride.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a piperazine structure, R₃ is methyl, and X is iodide.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a piperazine structure, R₃ is methyl, and X is bromide.

For any of the preceding n values, which can be 2, 3, 4, or 5, and any of the preceding m values, which can be 9, 10, 11, 12, 13, 14, 15, 16, 17, 18, 19, or 20, other compounds provided by the present disclosure are compounds of Formula I, wherein R₃ or R₆ is methyl.

For any of the preceding n values, which can be 2, 3, 4, or 5, and any of the preceding m values, which can be 9, 10, 11, 12, 13, 14, 15, 16, 17, 18, 19, or 20, other compounds provided by the present disclosure are compounds of Formula I, wherein R₃ or R₆ is methyl, and X is chloride.

For any of the preceding n values, which can be 2, 3, 4, or 5, and any of the preceding m values, which can be 9, 10, 11, 12, 13, 14, 15, 16, 17, 18, 19, or 20, other compounds provided by the present disclosure are compounds of Formula I, wherein R₃ or R₆ is methyl, and X is iodide.

For any of the preceding n values, which can be 2, 3, 4, or 5, and any of the preceding m values, which can be 9, 10, 11, 12, 13, 14, 15, 16, 17, 18, 19, or 20, other compounds provided by the present disclosure are compounds of Formula I, wherein R₃ or R₆ is methyl, and X is bromide.

For any of the preceding n values, which can be 2, 3, 4, or 5, and any of the preceding m values, which can be 9, 10, 11, 12, 13, 14, 15, 16, 17, 18, 19, or 20, compounds provided by the present disclosure include compounds of Formula I, wherein the counterion X is chloride.

For any of the preceding n values, which can be 2, 3, 4, or 5, and any of the preceding m values, which can be 9, 10, 11, 12, 13, 14, 15, 16, 17, 18, 19, or 20, compounds provided by the present disclosure include compounds of Formula I, wherein the counterion X is iodide.

For any of the preceding n values, which can be 2, 3, 4, or 5, and any of the preceding m values, which can be 9, 10, 11, 12, 13, 14, 15, 16, 17, 18, 19, or 20, compounds provided by the present disclosure include compounds of Formula I, wherein the counterion X is bromide.

For any of the preceding n values, which can be 2, 3, 4, or 5, and any of the preceding m values, which can be 9, 10, 11, 12, 13, 14, 15, 16, 17, 18, 19, or 20, compounds provided by the present disclosure include compounds of Formula I, wherein R₆ is oxygen.

For any of the preceding n values, which can be 2, 3, 4, or 5, and any of the preceding m values, which can be 9, 10, 11, 12, 13, 14, 15, 16, 17, 18, 19, or 20, compounds provided by the present disclosure include compounds of Formula I, wherein R₆ is oxygen and X is chloride.

For any of the preceding n values, which can be 2, 3, 4, or 5, and any of the preceding m values, which can be 9, 10, 11, 12, 13, 14, 15, 16, 17, 18, 19, or 20, compounds provided by the present disclosure include compounds of Formula I, wherein R₆ is oxygen and X is iodide.

For any of the preceding n values, which can be 2, 3, 4, or 5, and any of the preceding m values, which can be 9, 10, 11, 12, 13, 14, 15, 16, 17, 18, 19, or 20, compounds provided by the present disclosure include compounds of Formula I, wherein R₆ is oxygen and X is bromide.

For any of the preceding n values, which can be 2, 3, 4, or 5, and any of the preceding m values, which can be 9, 10, 11, 12, 13, 14, 15, 16, 17, 18, 19, or 20, compounds provided by the present disclosure include compounds of Formula I, wherein R₆ is one of a C₁-C₆ alkyl, optionally substituted with hydroxyl.

For any of the preceding n values, which can be 2, 3, 4, or 5, and any of the preceding m values, which can be 9, 10, 11, 12, 13, 14, 15, 16, 17, 18, 19, or 20, compounds provided by the present disclosure include compounds of Formula I, wherein R₆ is one of a C₁-C₆ alkyl, optionally substituted with hydroxyl and X is chloride.

For any of the preceding n values, which can be 2, 3, 4, or 5, and any of the preceding m values, which can be 9, 10, 11, 12, 13, 14, 15, 16, 17, 18, 19, or 20, compounds provided by the present disclosure include compounds of Formula I, wherein R₆ is one of a C₁-C₆ alkyl, optionally substituted with hydroxyl and X is iodide.

For any of the preceding n values, which can be 2, 3, 4, or 5, and any of the preceding m values, which can be 9, 10, 11, 12, 13, 14, 15, 16, 17, 18, 19, or 20, compounds provided by the present disclosure include compounds of Formula I, wherein R₆ is one of a C₁-C₆ alkyl, optionally substituted with hydroxyl and X is bromide.

For any of the preceding n values, which can be 2, 3, 4, or 5, and any of the preceding m values, which can be 9, 10, 11, 12, 13, 14, 15, 16, 17, 18, 19, or 20, compounds provided by the present disclosure include compounds of Formula I, wherein the counterion W is Na.

Compounds provided by the present disclosure are compounds of Formula I, wherein Y is of Formula (1-1), where the compound is selected from

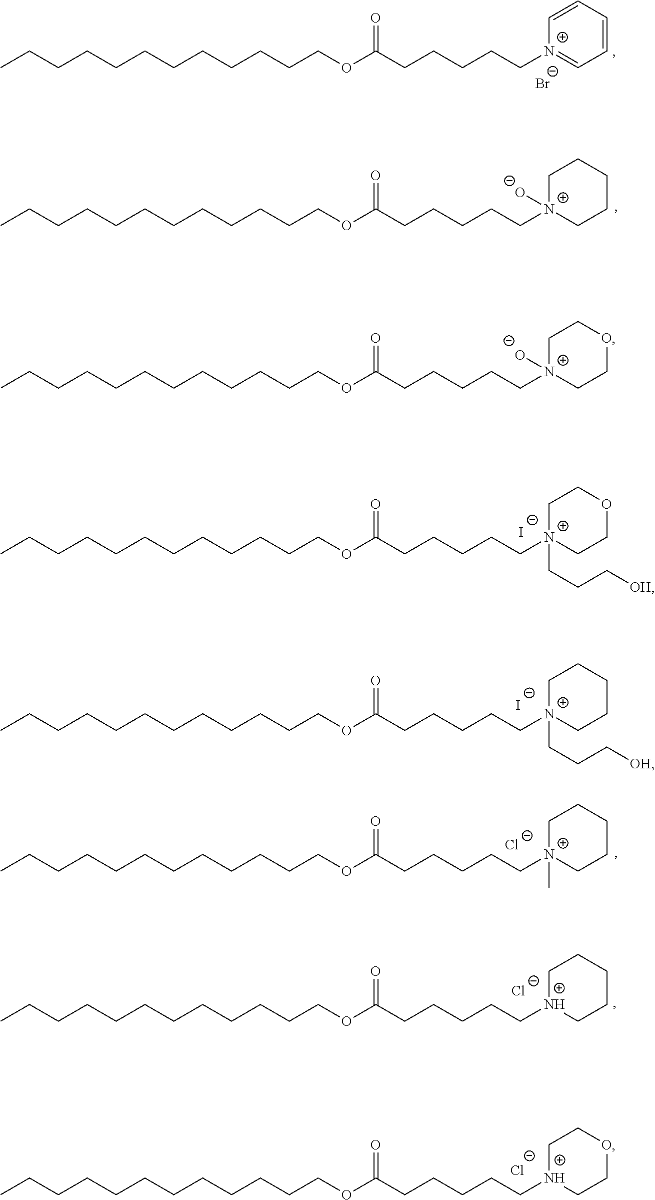

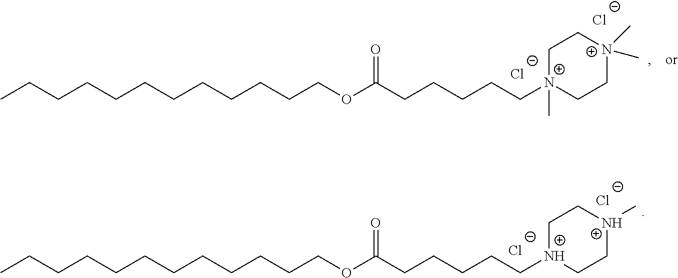

Compounds provided by the present disclosure are compounds of Formula I, where Y is of Formula (1-2), where the compound is selected from

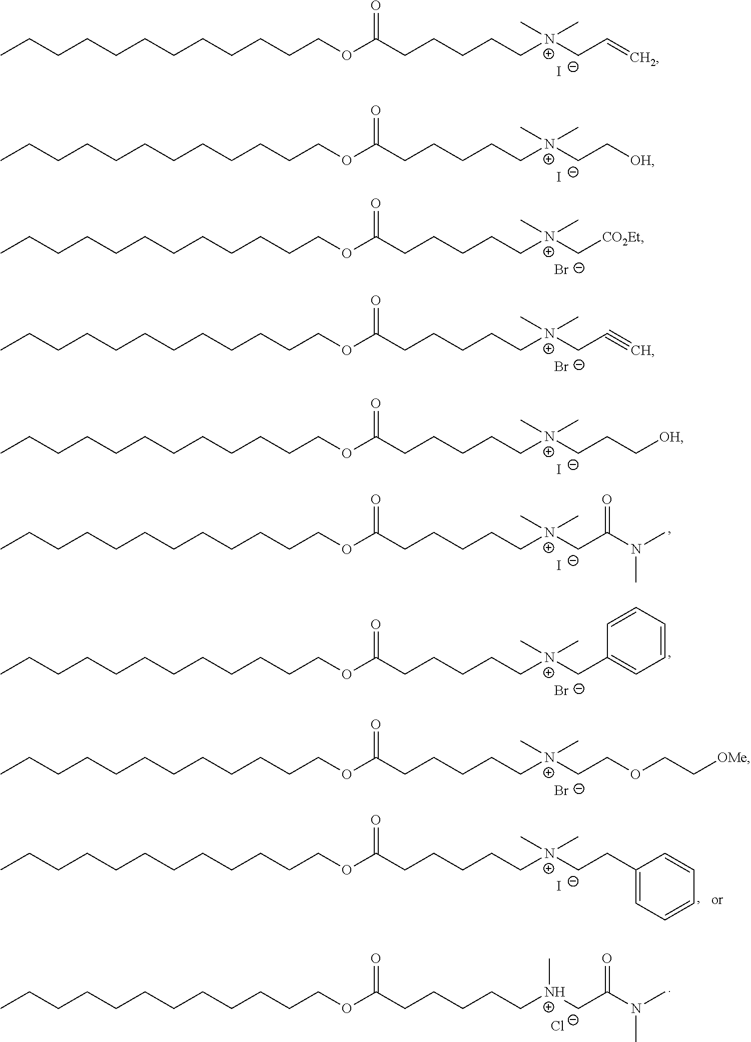

Compounds provided by the present disclosure are compounds of Formula I, where Y is of Formula (1-3), where the compound is selected from

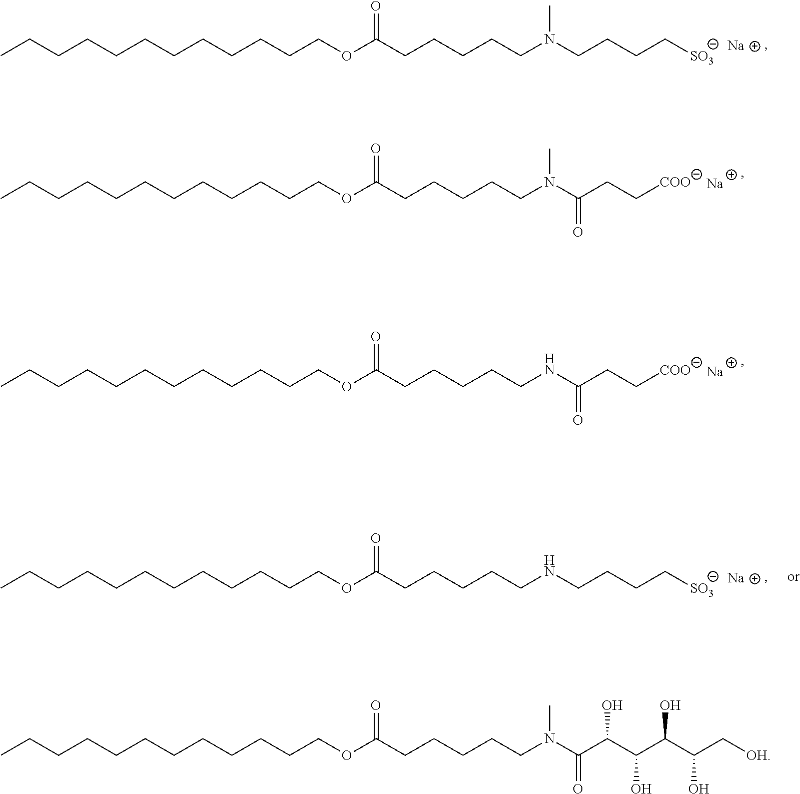

The present disclosure provides a compound of Formula II, wherein the compound of Formula II can be a salt, below:

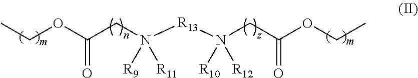

R₉, R₁₀, may be the same or different, and are selected from the group consisting of hydrogen and C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate, optionally, R₉ and R₁₀ are linked or otherwise connected together to form a 4 to 20 membered ring that may be substituted or unsubstituted, saturated or unsaturated; optionally the ring may include one or more of oxygen, nitrogen, or sulfur atoms or groups that include at least one of these atoms at any suitable position in the ring, and the ring may be optionally substituted with one or more substituents selected from the group consisting of one of C₁-C₆ alkyl, hydroxyl, amino, amido, sulfonyl, sulfonate, carbonyl, carboxyl, and carboxylate, wherein the ring formed from R₉ and Ru is optionally linked and/or fused to a further ring;
R₁₁, R₁₂ are optionally present, and if present are the same or different, and are selected from the group consisting of hydrogen and C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate;
if R₁₁ and R₁₂ are present, a counter ion Z is selected from the group consisting of chloride, bromide, and iodide.
R₁₃ is selected from the group consisting of C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate;
Z is selected from the group consisting of chloride, bromide, and iodide.
n and z are, independently, an integer in the range of 2 to 5; and
m is an integer in the range of 9 to 20.

Compounds provided by the present disclosure are compounds of Formula II, wherein n is 2, 3, 4 or 5, preferably 5.

Compounds provided by the present disclosure are compounds of Formula II, wherein z is 2, 3, 4, or 5, preferably 5.

Compounds provided by the present disclosure are compounds of Formula II, wherein m is 9, 10, 11, 12, 13, 14, 15, 16, 17, 18, 19, or 20, preferably 11.

Compounds provided by the present disclosure are compounds of Formula II, wherein the compound is selected from:

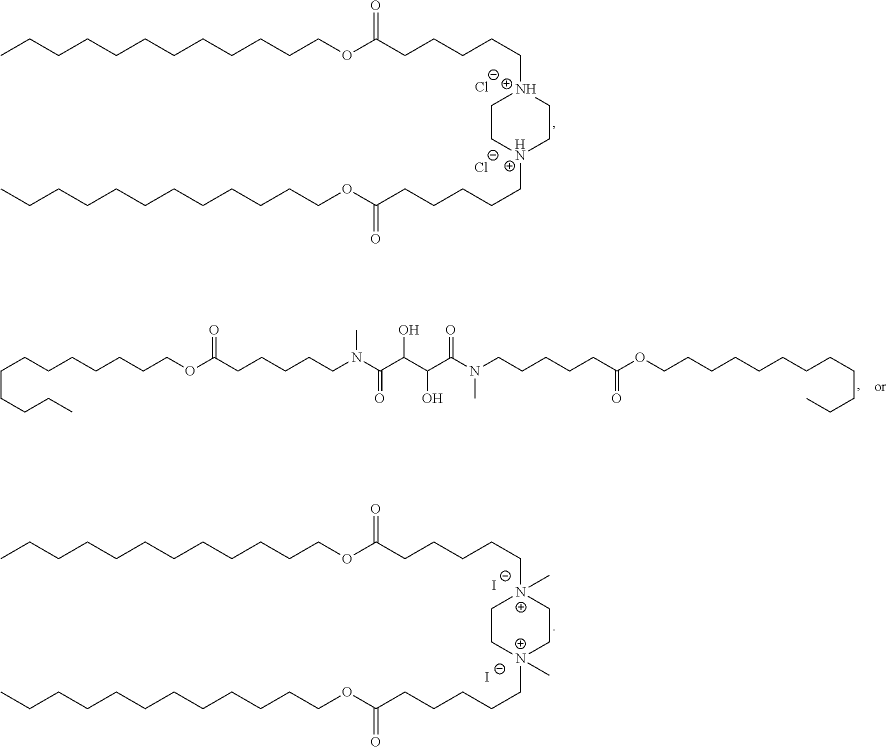

One specific compound provided by the present disclosure and referred to herein as Surfactant 1 is 1-(6-(dodecyloxy)-6-oxohexyl)pyridin-1-ium bromide:

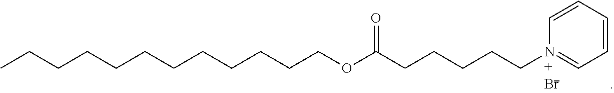

One specific compound provided by the present disclosure and referred to herein as Surfactant 2 is 4-(6-(dodecyloxy)-6-oxohexyl)-4-(3-hydroxypropyl)morpholin-4-ium iodide:

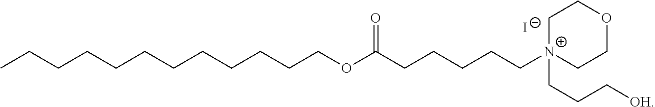 [SMILES: CCCCCCCCCCCCOC(=O)CCCCC[N+]1(CCCO)CCOCC1 ; [I-]]

One specific compound provided by the present disclosure and referred to herein as Surfactant 3 is 1-(6-(dodecyloxy)-6-oxohexyl)piperidin-1-ium chloride:

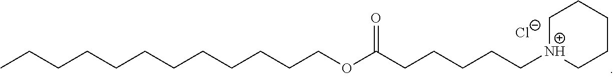 [SMILES: CCCCCCCCCCCCOC(=O)CCCCC[NH+]1CCCCC1 ; [Cl-]]

One specific compound provided by the present disclosure and referred to herein as Surfactant 4 is 4-(6-(dodecyloxy)-6-oxohexyl)morpholin-4-ium chloride:

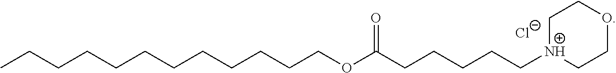 [SMILES: CCCCCCCCCCCCOC(=O)CCCCC[NH+]1CCOCC1 ; [Cl-]]

One specific compound provided by the present disclosure and referred to herein as Surfactant 5 is 1-(6-(dodecyloxy)-6-oxohexyl)-4-methylpiperazine-1,4-diium dichloride:

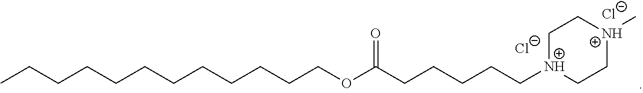 [SMILES: [Cl-] ; CCCCCCCCCCCCOC(=O)CCCCC[NH+]1CC[NH+](C)CC1 ; [Cl-]]

One specific compound provided by the present disclosure and referred to herein as Surfactant 6 is 4-(6-(dodecyloxy)-6-oxohexyl)morpholine 4-oxide:

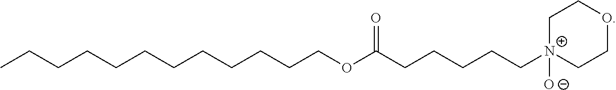 [SMILES: CCCCCCCCCCCCOC(=O)CCCCC[N+]1([O-])CCOCC1]

One specific compound provided by the present disclosure and referred to herein as Surfactant 7 is N-allyl-6-(dodecyloxy)-N,N-dimethyl-6-oxohexan-1-aminium iodide:

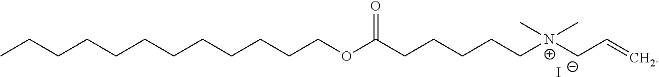 [SMILES: C=CC[N+](C)(C)CCCCCC(=O)OCCCCCCCCCCCC ; [I-]]

One specific compound provided by the present disclosure and referred to herein as Surfactant 8 is 6-(dodecyloxy)-N-(2-hydroxyethyl)-N,N-dimethyl-6-oxohexan-1-aminium iodide:

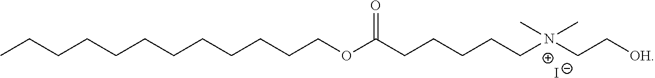 [SMILES: CCCCCCCCCCCCOC(=O)CCCCC[N+](C)(C)CCO ; [I-]]

One specific compound provided by the present disclosure and referred to herein as Surfactant 9 is 6-(dodecyloxy)-N-(2-ethoxy-2-oxoethyl)-N,N-dimethyl-6-oxohexan-1-aminium bromide:

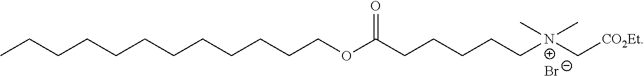 [SMILES: CCCCCCCCCCCCOC(=O)CCCCC[N+](C)(C)CC(=O)OCC ; [Br-]]

One specific compound provided by the present disclosure and referred to herein as Surfactant 10 is 6-(dodecyloxy)-N,N-dimethyl-6-oxo-N-(prop-2-yn-1-yl)hexan-1-aminium bromide:

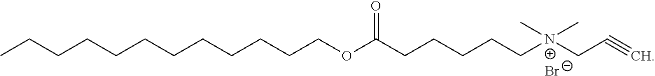 [SMILES: C#CC[N+](C)(C)CCCCCC(=O)OCCCCCCCCCCCC ; [Br-]]

One specific compound provided by the present disclosure and referred to herein as Surfactant 11 is 6-(dodecyloxy)-N-(3-hydroxypropyl)-N,N-dimethyl-6-oxohexan-1-aminium iodide:

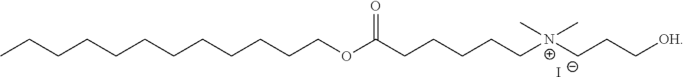 [SMILES: CCCCCCCCCCCCOC(=O)CCCCC[N+](C)(C)CCCO ; [I-]]

One specific compound provided by the present disclosure and referred to herein as Surfactant 12 is N-(2-(dimethylamino)-2-oxoethyl)-6-(dodecyloxy)-N,N-dimethyl-6-oxohexan-1-aminium iodide:

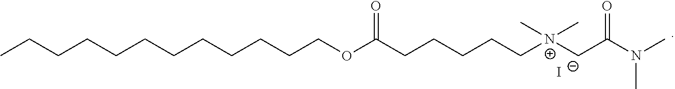 [SMILES: CCCCCCCCCCCCOC(=O)CCCCC[N+](C)(C)CC(=O)N(C)C ; [I-]]

One specific compound provided by the present disclosure and referred to herein as Surfactant 13 is N-benzyl-6-(dodecyloxy)-N,N-dimethyl-6-oxohexan-1-aminium bromide:

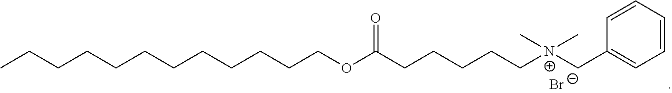 [SMILES: CCCCCCCCCCCCOC(=O)CCCCC[N+](C)(C)Cc1ccccc1 ; [Br-]]

One specific compound provided by the present disclosure and referred to herein as Surfactant 14 is 6-(dodecyloxy)-N-(2-(2-methoxyethoxy)ethyl)-N,N-dimethyl-6-oxohexan-1-aminium bromide:

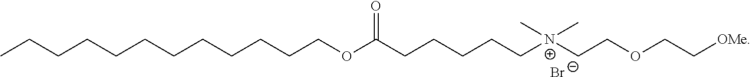 [SMILES: CCCCCCCCCCCCOC(=O)CCCCC[N+](C)(C)CCOCCOC ; [Br-]]

One specific compound provided by the present disclosure and referred to herein as Surfactant 15 is 6-(dodecyloxy)-N,N-dimethyl-6-oxo-N-phenethylhexan-1-aminium iodide:

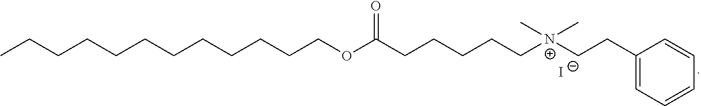 [SMILES: CCCCCCCCCCCCOC(=O)CCCCC[N+](C)(C)CCc1ccccc1 ; [I-]]

One specific compound provided by the present disclosure and referred to herein as Surfactant 16 is N-(2-(dimethylamino)-2-oxoethyl)-6-(dodecyloxy)-N-methyl-6-oxohexan-1-aminium chloride:

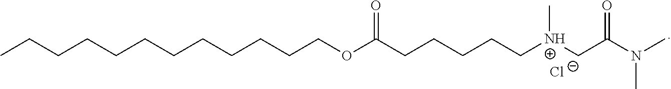 [SMILES: CCCCCCCCCCCCOC(=O)CCCCC[NH+](C)CC(=O)N(C)C ; [Cl-]]

One specific compound provided by the present disclosure and referred to herein as Surfactant 17 is sodium 4-((6-(dodecyloxy)-6-oxohexyl)(methyl)amino)butane-1-sulfonate:

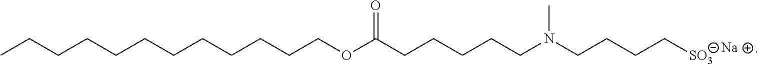

One specific compound provided by the present disclosure and referred to herein as Surfactant 18 is sodium 5-((6-(dodecyloxy)-6-oxohexyl)(methyl)amino)pentanoate:

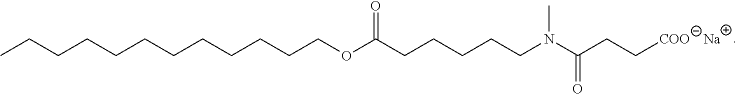

One specific compound provided by the present disclosure and referred to herein as Surfactant 19 is sodium 5-((6-(dodecyloxy)-6-oxohexyl)amino)pentanoate:

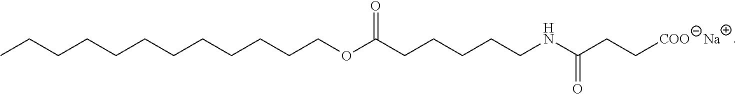

One specific compound provided by the present disclosure and referred to herein as Surfactant 20 is sodium 4-((6-(dodecyloxy)-6-oxohexyl)amino)butane-1-sulfonate:

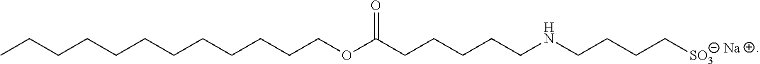

One specific compound provided by the present disclosure and referred to herein as Surfactant 21 is dodecyl 6-(2,3,4,5,6-pentahydroxy-N-methylhexanamido)hexanoate:

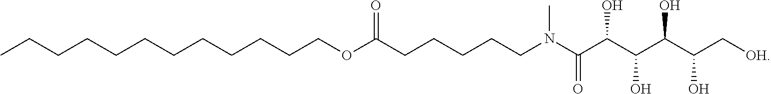 [SMILES: CCCCCCCCCCCCOC(=O)CCCCCN(C)C(=O)[C@H](O)[C@@H](O)[C@@H](O)[C@@H](O)CO]

One specific compound provided by the present disclosure and referred to herein as Surfactant 22 is 1,4-bis(6-(dodecyloxy)-6-oxohexyl)piperazine-1,4-diium dichloride:

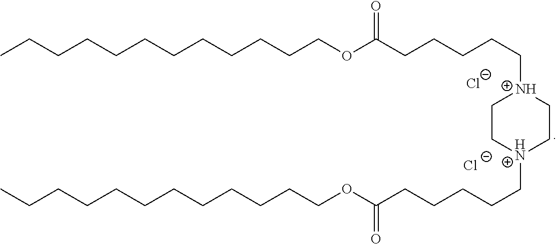

One specific compound provided by the present disclosure and referred to herein as Surfactant 23 is didodecyl 6,6′-((2,3-dihydroxysuccinyl)bis(methylazanediyl))dihexanoate:

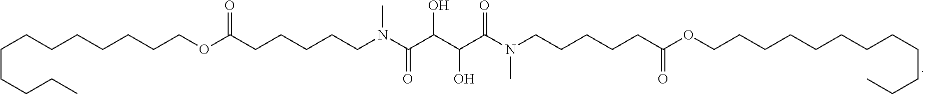 [SMILES: CCCCCCCCCCCCOC(=O)CCCCCN(C)C(=O)C(O)C(O)C(=O)N(C)CCCCCC(=O)OCCCCCCCCCCCC]

One specific compound provided by the present disclosure and referred to herein as Surfactant 24 is 1,4-bis(6-(dodecyloxy)-6-oxohexyl)-1,4-dimethylpiperazine-1,4-diium diiodide:

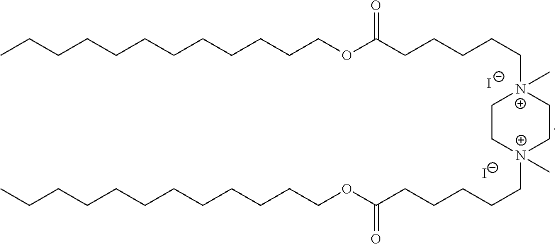 [SMILES: CCCCCCCCCCCCOC(=O)CCCCC[N+]1(C)CC[N+](C)(CCCCCC(=O)OCCCCCCCCCCCC)CC1 ; [I-] ; [I-]]

The above mentioned and other features of the disclosure, and the manner of attaining them, will become more apparent and will be better understood by reference to the following description taken in conjunction with the accompanying drawings.

### BRIEF DESCRIPTION OF THE DRAWINGS

FIG. 1 shows a plot of surface tension versus concentration for Surfactant 1 as described in Example 1a.

FIG. 2 shows a plot of surface tension versus concentration for Surfactant 2 as described in Example 2a.

FIG. 3 shows a plot of surface tension versus concentration for Surfactant 3 as described in Example 3a.

FIG. 4 shows a plot of surface tension versus concentration for Surfactant 4 as described in Example 4a.

FIG. 5 shows a plot of surface tension versus concentration for Surfactant 5 as described in Example 5a.

FIG. 6 shows a plot of surface tension versus concentration for Surfactant 6 as described in Example 6a.

FIG. 7 shows a plot of surface tension versus concentration for Surfactant 7 as described in Example 7a.

FIG. 8 shows a plot of surface tension versus concentration for Surfactant 8 as described in Example 8a.

FIG. 9 shows a plot of surface tension versus concentration for Surfactant 9 as described in Example 9a.

FIG. 10 shows a plot of surface tension versus concentration for Surfactant 10 as described in Example 10a.

FIG. 11 shows a plot of surface tension versus concentration for Surfactant 11 as described in Example 11a.

FIG. 12 shows a plot of surface tension versus concentration for Surfactant 12 as described in Example 12a.

FIG. 13 shows a plot of surface tension versus concentration for Surfactant 13 as described in Example 13a.

FIG. 14 shows a plot of surface tension versus concentration for Surfactant 14 as described in Example 14a.

FIG. 15 shows a plot of surface tension versus concentration for Surfactant 16 as described in Example 16a.

FIG. 16 shows a plot of surface tension versus concentration for Surfactant 17 as described in Example 17a.

FIG. 17 shows a plot of surface tension versus concentration for Surfactant 18 as described in Example 18a.

FIG. 18 shows a plot of surface tension versus concentration for Surfactant 19 as described in Example 19a.

FIG. 19 shows a plot of surface tension versus concentration for Surfactant 20 as described in Example 20a.

FIG. 20 shows a plot of surface tension versus concentration for Surfactant 21 as described in Example 21a.

FIG. 21 shows a plot of surface tension versus concentration for Surfactant 22 as described in Example 22a.

FIG. 22 shows a plot of surface tension versus concentration for Surfactant 23 as described in Example 23a.

FIG. 23 shows a plot of surface tension versus concentration for Surfactant 24 as described in Example 24a.

### DETAILED DESCRIPTION

As used herein, the phrase “within any range defined between any two of the foregoing values” means that any range may be selected from any two of the values listed prior to such phrase regardless of whether the values are in the lower part of the listing or in the higher part of the listing. For example, a pair of values may be selected from two lower values, two higher values, or a lower value and a higher value.

As used herein, the phrase “surface-active” means that the associated compound is able to lower the surface tension of the medium in which it is dissolved, and/or the interfacial tension with other phases, and, accordingly, may be adsorbed at the liquid/vapor and/or other interfaces. For the purposes of this disclosure, the surfactants of the invention are “surface active” compounds.

With respect terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error or minor adjustments made to optimize performance, for example. As an illustrative example, for scientific measures involving extensive manual preparations of reagents, when the amounts of such reagents used are small or even minute, and results are sensitive to small changes in terms of the amounts of reagents used, there would likely be a high potential for human errors, or when the instrumentations used are such that small changes in experimental procedures and reagent handling can cause changes in results, especially when the deviations can vary widely and not always reproducible, the term “about” used when reporting such results can be as high as plus or minus 15% of the stated value, for example, plus or minus 15% of the stated value, plus or minus 12% of the stated value, or plus or minus 10% of the stated value, or plus or minus 8% of the stated value, or plus or minus 6% of the stated value, or plus or minus 4% of the stated value, or even plus or minus 2% of the stated value and the like.

The present disclosure provides siloxane derivatives of amino acids. The amino acids may be naturally occurring or synthetic, or they may be obtained from ring-opening reactions of lactams, such as caprolactam. The compounds of the present disclosure have been shown to have surface-active properties, and can be used as surfactants including wetting agents, for example.

The present disclosure provides a compound of Formula I, wherein the compound of Formula I can be a salt, below:

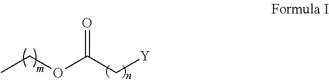

wherein Y is selected from formula (1-1), (1-2), or (1-3)

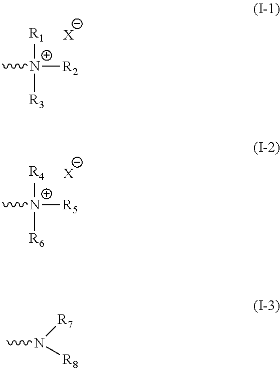

the wave-line denoting the connection to Formula I;
   wherein
R₁ and R₂ are each one of C₁-C₁₀ alkyl and are linked or otherwise connected together to form a 4 to 20 membered ring that may be substituted or unsubstituted, saturated or unsaturated; optionally the ring may include one or more of oxygen, nitrogen, or sulfur atoms or groups that include at least one of these atoms at any suitable position in the ring, and the ring may be optionally substituted with one or more substituents selected from the group consisting of one of C₁-C₆ alkyl, hydroxyl, amino, amido, sulfonyl, sulfonate, carbonyl, carboxyl, and carboxylate; wherein the ring formed from R₁ and R₂ is optionally linked and/or fused to a further ring;
R₃ is selected from the group consisting of hydrogen, C₁-C₆ alkyl, C₂-C₁₀ alkenyl, C₂-C₁₀ alkynyl, C₂-C₁₂ ester, hydroxyl, C₁-C₁₀ hydroxyl, aryl C₁-C₆ alkyl, C₂-C₁₂ alkoxy alkyl ether, C₁-C₆ alkyl phosphate, C₃-C₈ carboxylic acid, C₁-C₁₀ alkyl benzoic acid, oxygen, and sulfur;
R₄ and R₅ may be the same or different, and they are individually selected from the group consisting of hydrogen and C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate;
R₆ is and is selected from the group consisting of C₂-C₁₀ alkenyl, C₂-C₁₀ alkynyl, C₂-C₁₂ ester, hydroxyl, aryl C₁-C₆ alkyl, C₂-C₁₂ alkoxy alkyl ether, C₁-C₆ alkyl phosphate, C₃-C₈carboxylic acid, C₁-C₁₀alkyl benzoic acid, amino, amido, and sulfur; wherein R₆ may optionally be substituted with one or more substituents selected from the group consisting of aryl, heteroaryl, hydroxyl, amino, amido, sulfonyl, sulfonate, carbonyl, carboxyl, and carboxylate;
R₇ and R₈ may be the same or different, and are selected from the group consisting of hydrogen and C₁-C₆ alkyl, wherein the C₁-C₆ alkyl is substituted with one or more substituents selected from the group consisting of hydroxyl, amino, amido, sulfonyl, sulfonate, carbonyl, carboxyl, and carboxylate, or a salt thereof, wherein a counterion W associated with any of the hydroxyl, sulfonate, or carboxylate group is selected from Na, K or Ca;
X is selected from the group consisting of chloride, bromide, and iodide.
n is an integer in the range of 2 to 5; and
m is an integer in the range of 9 to 20.

The compound provided by the present disclosure is a compound of Formula I, wherein in R₁ and R₂ form a ring with 4 to 20, with 4 to 15, with 4 to 10, with 4 to 8, with 4, 5 or 6 atoms, preferably with 6 atoms.

For example, the structure of the formula (1-1) may include piperidine, pyridine, pyrimidine, pyrrole, imidazole, pyrazole, piperazine, or morpholine.

For example, structure of the formula (1-1) is selected from

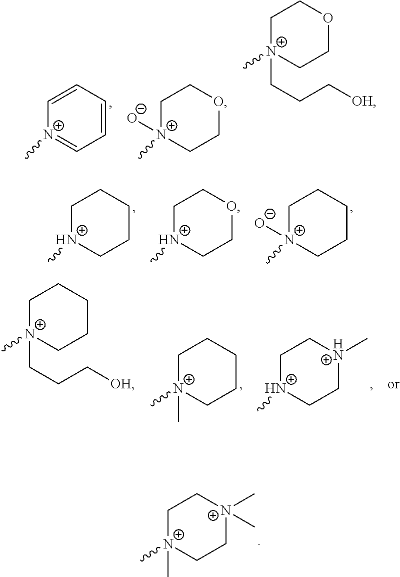

For example, structure of the formula (1-2) or (1-3) is selected from

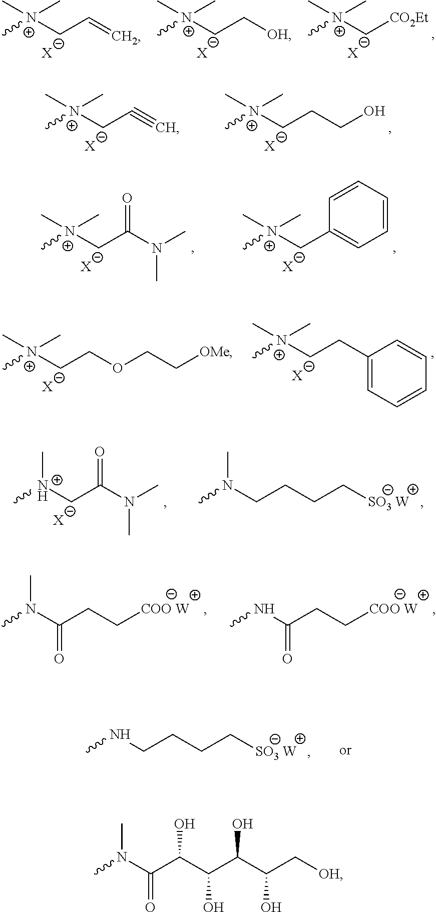

wherein
X is selected from the group consisting of chloride, bromide, and iodide, and
W is selected from the group consisting of sodium, potassium, or calcium.

Compounds provided by the present disclosure are compounds of Formula I, where n is 2, 3, 4, or 5, particularly wherein n is 5.

For any of the preceding n values, which can be 2, 3, 4, or 5, the compounds provided by the present disclosure are of Formula I, where m is 9, 10, 11, 12, 13, 14, 15, 16, 17, 18, 19, or 20, particularly wherein m is 11.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a morpholine structure.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a morpholine structure, R₃ is methyl, and X is chloride.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a morpholine structure, R₃ is methyl, and X is iodide.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a morpholine structure, R₃ is methyl, and X is bromide.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a piperidine structure.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a piperidine structure, R₃ is methyl, and X is chloride.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a piperidine structure, R₃ is methyl, and X is iodide.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a piperidine structure, R₃ is methyl, and X is bromide.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a piperazine structure.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a piperazine structure, R₃ is methyl, and X is chloride.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a piperazine structure, R₃ is methyl, and X is iodide.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure are compounds of Formula I, wherein R₁ and R₂ form a piperazine structure, R₃ is methyl, and X is bromide.

For any of the preceding n values, which can be 2, 3, 4, or 5, and any of the preceding m values, which can be 9, 10, 11, 12, 13, 14, 15, 16, 17, 18, 19, or 20, other compounds provided by the present disclosure are compounds of Formula I, wherein R₃ or R₆ is methyl.

For any of the preceding n values, which can be 2, 3, 4, or 5, and any of the preceding m values, which can be 9, 10, 11, 12, 13, 14, 15, 16, 17, 18, 19, or 20, other compounds provided by the present disclosure are compounds of Formula I, wherein R₃ or R₆ is methyl and X is chloride.

For any of the preceding n values, which can be 2, 3, 4, or 5, and any of the preceding m values, which can be 9, 10, 11, 12, 13, 14, 15, 16, 17, 18, 19, or 20, other compounds provided by the present disclosure are compounds of Formula I, wherein R₃ or R₆ is methyl and X is iodide.

For any of the preceding n values, which can be 2, 3, 4, or 5, and any of the preceding m values, which can be 9, 10, 11, 12, 13, 14, 15, 16, 17, 18, 19, or 20, other compounds provided by the present disclosure are compounds of Formula I, wherein R₃ or R₆ is methyl and X is bromide.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure include compounds of Formula I wherein the counterion X is chloride.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure include compounds of Formula I wherein the counterion X is iodide.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure include compounds of Formula I wherein the counterion X is bromide.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure include compounds of Formula I, wherein R₆ is oxygen.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure include compounds of Formula I, wherein R₆ is oxygen and X is chloride.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure include compounds of Formula I, wherein R₆ is oxygen and X is iodide.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure include compounds of Formula I, wherein R₆ is oxygen and X is bromide.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure include compounds of Formula I, wherein R₆ is one of C₁-C₆ alkyl, optionally substituted with hydroxyl.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure include compounds of Formula I, wherein R₆ is one of C₁-C₆ alkyl, optionally substituted with hydroxyl and X is chloride.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure include compounds of Formula I, wherein R₆ is one of C₁-C₆ alkyl, optionally substituted with hydroxyl and X is iodide.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure include compounds of Formula I, wherein R₆ is one of C₁-C₆ alkyl, optionally substituted with hydroxyl and X is bromide.

For any of the preceding n values, which can be 2, 3, 4, or 5, and any of the preceding m values, which can be 9, 10, 11, 12, 13, 14, 15, 16, 17, 18, 19, or 20, the compounds provided by the present disclosure include compounds of Formula I wherein the counterion W is Na.

Compounds provided by the present disclosure are compounds of Formula I, where Y is of Formula (1-1), where the compound is selected from

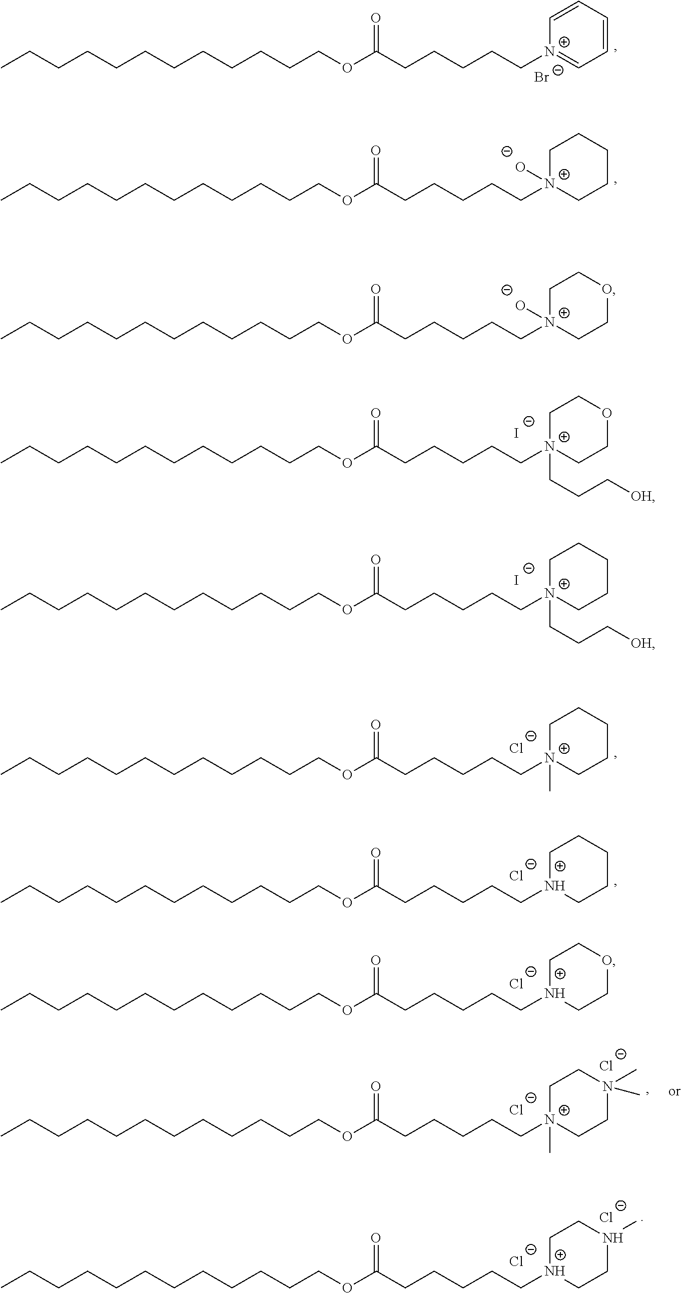

Compounds provided by the present disclosure are compounds of Formula I, where Y is of Formula (1-2), where the compound is selected from

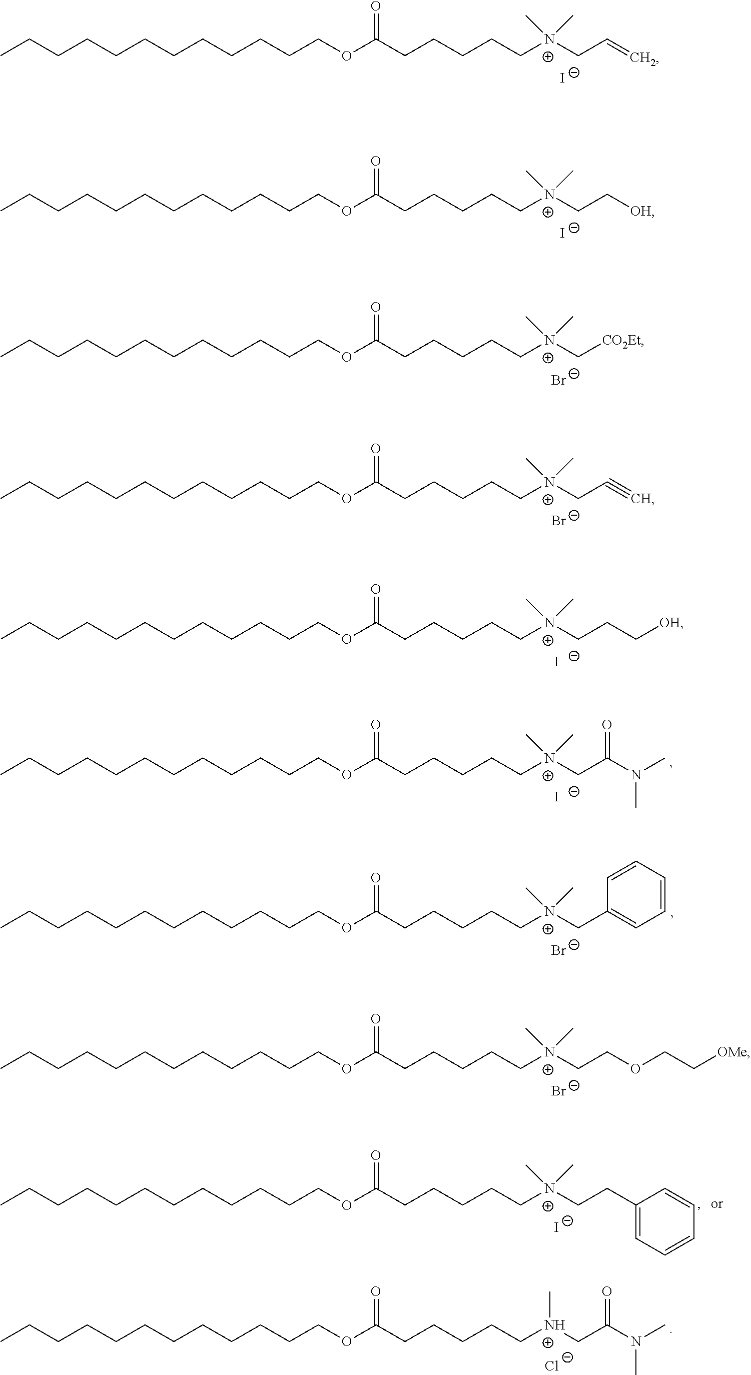

Compounds provided by the present disclosure are compounds of Formula 1, where Y is of Formula (1-3), where the compound is selected from

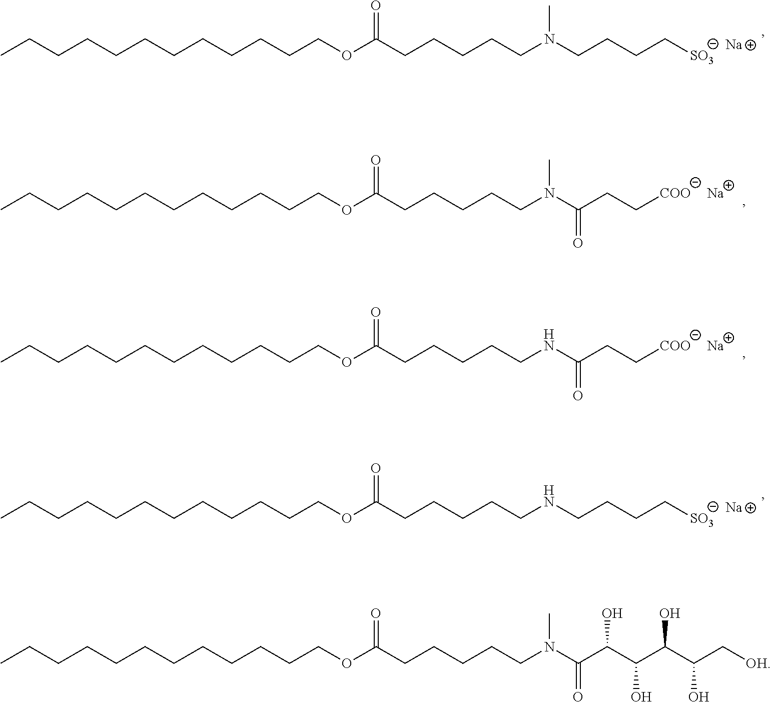

The present disclosure provides a compound of Formula II, wherein the compound of Formula II can be a salt, below:

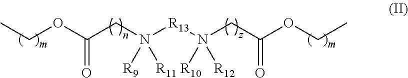

R₉, R₁₀, may be the same or different, and are selected from the group consisting of hydrogen and C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate, optionally, R₉ and R₁₀ are linked or otherwise connected together to form a 4 to 20 membered ring that may be substituted or unsubstituted, saturated or unsaturated, optionally the ring may include one or more of oxygen, nitrogen, or sulfur atoms or groups that include at least one of these atoms at any suitable position in the ring, and the ring may be optionally substituted with one or more substituents selected from the group consisting of any of C₁-C₆ alkyl, hydroxyl, amino, amido, sulfonyl, sulfonate, carbonyl, carboxyl, and carboxylate, wherein the ring formed from R₉ and R₁₀ is optionally linked and/or fused to a further ring;
R₁₁, R₁₂ are optionally present, and if present are the same or different, and are selected from the group consisting of hydrogen and C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate;
if R₁₁ and R₁₂ are present, a counter ion Z is selected from the group consisting of chloride, bromide, and iodide.
R₁₃ is selected from the group consisting of C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate;
Z is selected from the group consisting of chloride, bromide, and iodide.
n and z are, independently, an integer in the range of 2 to 5; and
m is an integer in the range of 9 to 20.

Compounds provided by the present disclosure are compounds of Formula II, wherein n is 2, 3, 4 or 5, preferably n is 5.

Compounds provided by the present disclosure are compounds of Formula II, wherein z is 2, 3, 4 or 5, preferably z is 5.

Compounds provided by the present disclosure are compounds of Formula II, wherein m is 9, 10, 11, 12, 13, 14, 15, 16, 17, 18, 19, or 20, preferably where m is 11.

Compounds provided by the present disclosure are compounds of Formula II, wherein the compound is selected from:

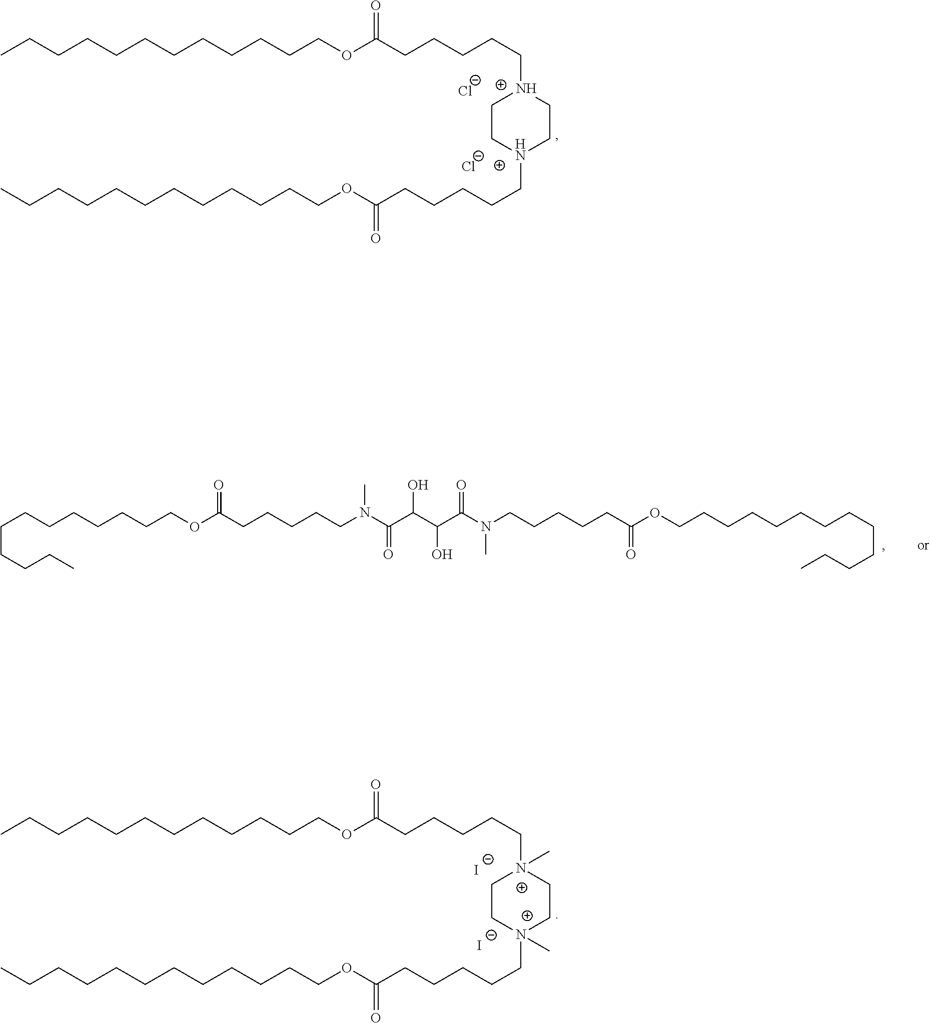

As used herein, the phrase “n may be an integer in the range of 1 to 12” means that n may be equal to 1, 2, 3, 4, 5, 6, 7, 8, 9, 10, 11, or 12, or within any range including any two of the foregoing as endpoints. Thus, n may be an integer selected from 1 to 8, from 2 to 6, from 3 to 6, 4 to 6, or n may be 5.

As used herein, the phrase “C₁-C₁₀ alkyl” means a straight chain or branched alkyl group containing 1, 2, 3, 4, 5, 6, 7, 8, 9 or 10 carbons or within any range including any two of the foregoing as endpoints. The alkyl groups may be C₁-C₆ alky groups. The phrase “C₁-C₆ alkyl” means a straight chain or branched alkyl group containing 1, 2, 3, 4, 5, or 6 carbons or within any range including any two of the foregoing as endpoints.

As used herein, the phrase “heteroalkyl” means a “C₁-C₆ alkyl,” as defined herein, wherein the alkyl group has at least one heteroatom (e.g., nitrogen, oxygen, sulfur, phosphorous, chlorine, bromine, and iodine).

As used herein, the phrase “C₂-C₁₀ alkenyl” means a straight chain or branched alkenyl group containing 2, 3, 4, 5, 6, 7, 8, 9, or 10 carbons or within any range including any two of the foregoing as endpoints.

As used herein, the phrase “C₂-C₁₀ alkynyl” means a straight chain or branched alkynyl group containing 2, 3, 4, 5, 6, 7, 8, 9, or 10 carbons or within any range including any two of the foregoing as endpoints.

As used herein, the phrase “C₂-C₁₂ ester” means a straight chain or branched ester group having a total of 2, 3, 4, 5, 6, 7, 8, 9, 10, 11 or 12 carbons or within any range including any two of the foregoing as endpoints.

As used herein, the phrase “C₂-C₁₂ alkoxy alkyl ether” means a straight chain or branched alkoxy alkyl ether group having a total of 2, 3, 4, 5, 6, 7, 8, 9, 10, 11 or 12 carbons or within any range including any two of the foregoing as endpoints.

As used herein, the phrase “hydroxyl” means —OH and “C₁-C₁₀ hydroxyl” means a hydroxyl attached to a straight chain or branched alkyl group containing 1, 2, 3, 4, 5, 6, 7, 8, 9, or 10 carbons or within any range including any two of the foregoing as endpoints.

As used herein, the phrase “aryl” means a hydrocarbon aromatic group preferably comprising from 5 to 10 carbon atoms and including one or more fused rings.

As used herein, the phrase “heteroaryl” means an aryl group, as defined herein, wherein the aryl group has at least one heteroatom (e.g., nitrogen, oxygen, or sulfur) in the ring.

As used herein, the phrase “C₃-C₈ carboxylic acid” means a carboxylic acid group attached to a straight chain or branched alkyl group with a containing 3, 4, 5, 6, 7, or 8, carbons or within any range including any two of the foregoing as endpoints.

As used herein, the phrase “C₁-C₁₀ alkyl benzoic acid” means a benzoic acid group attached to a straight chain or branched alkyl group containing 1, 2, 3, 4, 5, 6, 7, 8, 9, or 10 carbons or within any range including any two of the foregoing as endpoints.

As used herein, an amino group is —N(R₁₄)₂ where R₁₄ is hydrogen or one of C₁ to C₆ alkyl.

As used herein, an amido group is —C(O)NR₁₄— or —NR₁₄C(O)— where R₁₄ is hydrogen or one of C₁ to C₆ alkyl.

As used herein, a sulfonyl group is —SO₂R₁₄ where R₁₄ is hydrogen or one of C₁ to C₆ alkyl.

As used herein, a sulfonate group is —SO₃R₁₄ where R₁₄ is hydrogen or one of C₁ to C₆ alkyl.

As used herein, a carbonyl group is —COR₁₄ where R₁₄ is hydrogen or one of C₁ to C₆ alkyl.

As used herein, a carboxyl group is —CO₂H.

As used herein, a carboxylate group is —CO₂R₁₄ where R₁₄ is hydrogen or one of C₁ to C₆ alkyl.

For the purposes of this disclosure, the compound of Formula (I), wherein the compound of Formula I can be a salt, below:

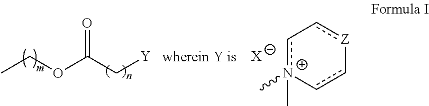

R₁₅ is either not present, or an oxygen, hydrogen, one of C₁-C₆ alkyl, or one of C₁-C₆ heteroalkyl;
Z is 0, S, NR₁₆R₁₇, or CR₁₈R₁₉
n is an integer in the range of 2 to 5 and m is an integer in the range of 9 to 20;
X is selected from the group consisting of chloride, bromide, and iodide; and
R₁₆ and R₁₇ are each independently hydrogen or one of C₁-C₆ alkyl;
R₁₈ and R₁₉ are each independently either not present, or a hydrogen, or one of C₁-C₆ alkyl;
[symbol] are single bonds or double bonds; and
wherein the ring may be optionally substituted with one or more substituents selected from the group consisting of one of C₁-C₆ alkyl, hydroxyl, amino, amido, sulfonyl, sulfonate, carbonyl, carboxyl, and carboxylate.

For the purposes of this disclosure, the compound of Formula (I), wherein the compound of Formula I can be a salt, below:

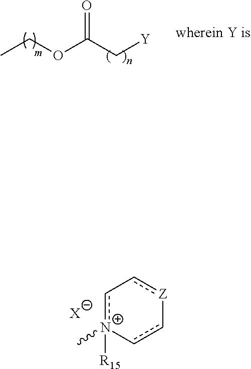

R₁₅ is oxygen, hydrogen, one of C₁-C₆ alkyl or one of C₁-C₆ heteroalkyl;
Z is O;
n is an integer in the range of 2 to 5 and m is an integer in the range of 9 to 20;
X is selected from the group consisting of chloride, bromide, and iodide;
[symbol] are single bonds; and
wherein the ring may be optionally substituted with one or more substituents selected from the group consisting of C₁-C₆ alkyl, hydroxyl, amino, amido, sulfonyl, sulfonate, carbonyl, carboxyl, and carboxylate.

For the purposes of this disclosure, the compound of Formula (I), wherein the compound of Formula I can be a salt, below:

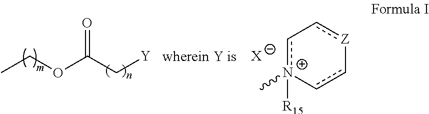

R₁₅ is either not present, or an oxygen, hydrogen, one of C₁-C₆ alkyl or one of C₁-C₆ heteroalkyl;
Z is CR₁₈R₁₉;
n is an integer in the range of 2 to 5 and m is an integer in the range of 9 to 20;
X is selected from the group consisting of chloride, bromide, and iodide;
R₁₈ and R₁₉ are each independently either not present, or a hydrogen, or one of C₁-C₆ alkyl; and
[symbol] are single bonds or double bonds;
wherein the ring may be optionally substituted with one or more substituents selected from the group consisting of C₁-C₆ alkyl, hydroxyl, amino, amido, sulfonyl, sulfonate, carbonyl, carboxyl, and carboxylate.

For the purposes of this disclosure, the compound of Formula (I), wherein the compound of Formula I can be a salt, below:

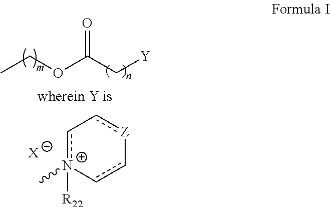

R₁₅ is oxygen, hydrogen, one of C₁-C₆ alkyl or one of C₁-C₆ heteroalkyl;
Z is NR₁₆R₁₇;
n is an integer in the range of 2 to 5 and m is an integer in the range of 9 to 20;
X is selected from the group consisting of chloride, bromide, and iodide;
R₁₆ and R₁₇ are each independently hydrogen or one of C₁-C₆ alkyl; and
[symbol] are single bonds; and
wherein the ring may be optionally substituted with one or more substituents selected from the group consisting of C₁-C₆ alkyl, hydroxyl, amino, amido, sulfonyl, sulfonate, carbonyl, carboxyl, and carboxylate.

For the purposes of this disclosure, the compound of Formula (I), wherein the compound of Formula I can be a salt, below:

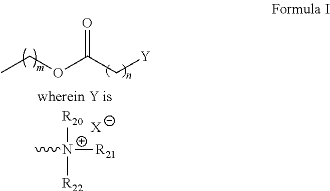

R₂₀ and R₂₁ are each independently methyl or hydrogen;
n is an integer in the range of 2 to 5 and m is an integer in the range of 9 to 20;
X is selected from the group consisting of chloride, bromide, and iodide;
R₂₂ is selected from the group consisting of C₂-C₁₀alkenyl, C₂-C₁₀alkynyl, C₂-C₁₂ ester, hydroxyl, aryl C₁-C₆ alkyl, C₂-C₁₂ alkoxy alkyl ether, C₁-C₆ alkyl phosphate, C₃-C₈ carboxylic acid, C₁-C₁₀alkyl benzoic acid, amino, amido, and sulfur; wherein R₂₂ may optionally be substituted with one or more substituents selected from the group consisting of aryl, heteroaryl, hydroxyl, amino, amido, sulfonyl, sulfonate, carbonyl, carboxyl, and carboxylate.

For the purposes of this disclosure, the compound of Formula (I), wherein the compound of Formula I can be a salt, below:

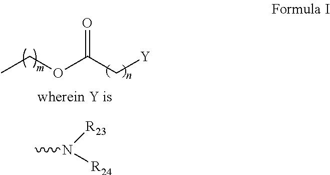

R₂₃ is methyl or hydrogen;
n is an integer from 2 to 5 and m is an integer from 9 to 20; and
R₂₄ is one of C₁-C₆ alkyl, wherein the C₁-C₆ alkyl is substituted with one or more substituents selected from the group consisting of hydroxyl, amino, amido, sulfonyl, sulfonate, carbonyl, carboxyl, and carboxylate, or a salt thereof, wherein a counterion W associated with any of the hydroxyl, sulfonate, or carboxylate group is selected from Na, K or Ca.

For the purposes of this disclosure, the compound of Formula (I), wherein the compound of Formula I can be a salt, below:

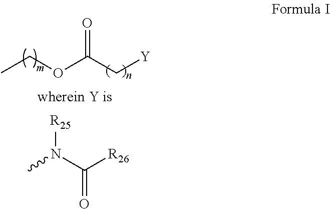

R₂₅ is hydrogen or one of C₁-C₆ alkyl;
R₂₆ is selected from the group consisting of hydrogen, C₁-C₆ alkyl, C₂-C₁₀ alkenyl, C₂-C₁₀ alkynyl, C₂-C₁₂ ester, hydroxyl, aryl C₁-C₆ alkyl, C₂-C₁₂ alkoxy alkyl ether, C₁-C₆ alkyl phosphate, C₁-C₆ alkyl sulfonates, C₃-C₈ carboxylic acid, C₁-C₁₀ alkyl benzoic acid, oxygen, and sulfur;
optionally when R₂₆ is one of C₁-C₆ alkyl, R₂₆ is substituted with one or more hydroxyl groups;
n is an integer in the range of 2 to 5, preferably n is 5; and
m is an integer in the range of 9 to 20, preferably m is 11.

For the purposes of this disclosure, the compound of Formula (II), wherein the compound of Formula II can be a salt, below:

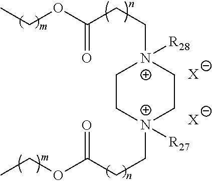

wherein
R₂₇ and R₂₈ are each independently hydrogen or one of C₁-C₆ alkyl;
n is an integer in the range of 1 to 4;
m is an integer in the range of 9 to 20; and
X is selected from the group consisting of chloride, bromide, and iodide.

One specific compound provided by the present disclosure and referred to herein as Surfactant 1 is 1-(6-(dodecyloxy)-6-oxohexyl)pyridin-1-ium bromide; having the formula:

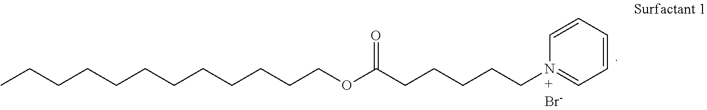

One specific compound provided by the present disclosure and referred to herein as Surfactant 2 is 4-(6-(dodecyloxy)-6-oxohexyl)-4-(3-hydroxypropyl)morpholin-4-ium iodide; having the formula:

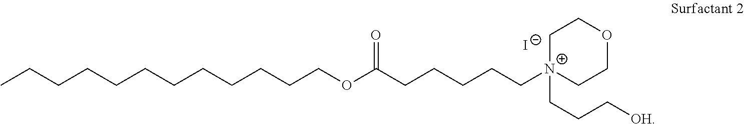 [SMILES: CCCCCCCCCCCCOC(=O)CCCCC[N+]1(CCCO)CCOCC1 ; [I-]]

One specific compound provided by the present disclosure and referred to herein as Surfactant 3 is 1-(6-(dodecyloxy)-6-oxohexyl)piperidin-1-ium chloride; having the formula:

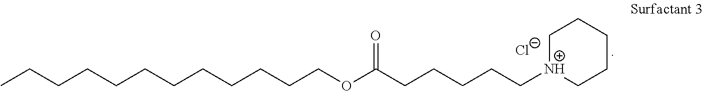 [SMILES: CCCCCCCCCCCCOC(=O)CCCCC[NH+]1CCCCC1 ; [Cl-]]

One specific compound provided by the present disclosure and referred to herein as Surfactant 4 is 4-(6-(dodecyloxy)-6-oxohexyl)morpholin-4-ium chloride; having the formula:

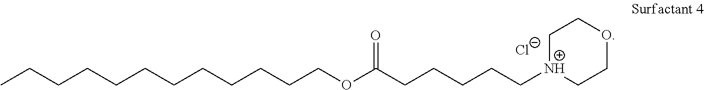 [SMILES: CCCCCCCCCCCCOC(=O)CCCCC[NH+]1CCOCC1 ; [Cl-]]

One specific compound provided by the present disclosure and referred to herein as Surfactant 5 is 1-(6-(dodecyloxy)-6-oxohexyl)-4-methylpiperazine-1,4-diium dichloride; having the formula:

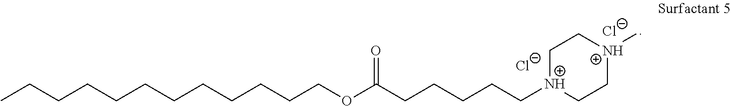 [SMILES: [Cl-] ; CCCCCCCCCCCCOC(=O)CCCCC[NH+]1CC[NH+](C)CC1 ; [Cl-]]

One specific compound provided by the present disclosure and referred to herein as Surfactant 6 is 4-(6-(dodecyloxy)-6-oxohexyl)morpholine 4-oxide; having the formula:

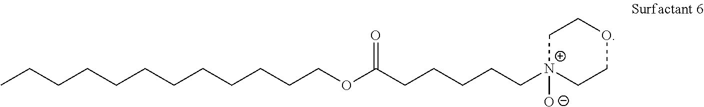

One specific compound provided by the present disclosure and referred to herein as Surfactant 7 is N-allyl-6-(dodecyloxy)-N,N-dimethyl-6-oxohexan-1-aminium iodide; having the formula:

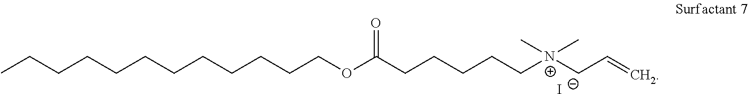 [SMILES: C=CC[N+](C)(C)CCCCCC(=O)OCCCCCCCCCCCC ; [I-]]

One specific compound provided by the present disclosure and referred to herein as Surfactant 8 is 6-(dodecyloxy)-N-(2-hydroxyethyl)-N,N-dimethyl-6-oxohexan-1-aminium iodide; having the formula:

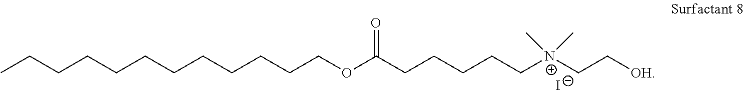 [SMILES: CCCCCCCCCCCCOC(=O)CCCCC[N+](C)(C)CCO ; [I-]]

One specific compound provided by the present disclosure and referred to herein as Surfactant 9 is 6-(dodecyloxy)-N-(2-ethoxy-2-oxoethyl)-N,N-dimethyl-6-oxohexan-1-aminium bromide; having the formula:

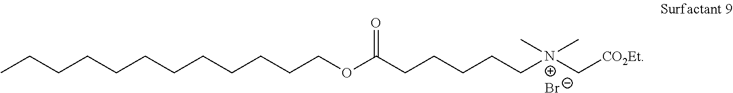 [SMILES: CCCCCCCCCCCCOC(=O)CCCCC[N+](C)(C)CC(=O)OCC ; [Br-]]

One specific compound provided by the present disclosure and referred to herein as Surfactant 10 is 6-(dodecyloxy)-N,N-dimethyl-6-oxo-N-(prop-2-yn-1-yl)hexan-1-aminium bromide; having the formula:

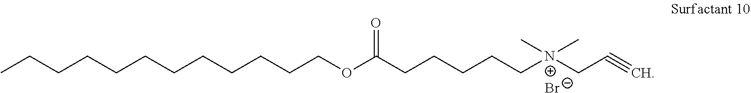 [SMILES: C#CC[N+](C)(C)CCCCCC(=O)OCCCCCCCCCCCC ; [Br-]]

One specific compound provided by the present disclosure and referred to herein as Surfactant 11 is 6-(dodecyloxy)-N-(3-hydroxypropyl)-N,N-dimethyl-6-oxohexan-1-aminium iodide; having the formula:

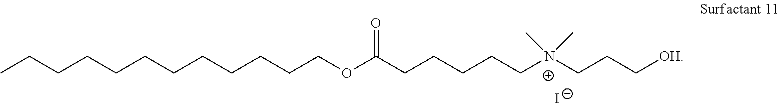 [SMILES: CCCCCCCCCCCCOC(=O)CCCCC[N+](C)(C)CCCO ; [I-]]

One specific compound provided by the present disclosure and referred to herein as Surfactant 12 is N-(2-(dimethylamino)-2-oxoethyl)-6-(dodecyloxy)-N,N-dimethyl-6-oxohexan-1-aminium iodide; having the formula:

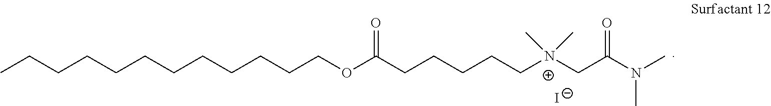 [SMILES: CCCCCCCCCCCCOC(=O)CCCCC[N+](C)(C)CC(=O)N(C)C ; [I-]]

One specific compound provided by the present disclosure and referred to herein as Surfactant 13 is N-benzyl-6-(dodecyloxy)-N,N-dimethyl-6-oxohexan-1-aminium bromide; having the formula:

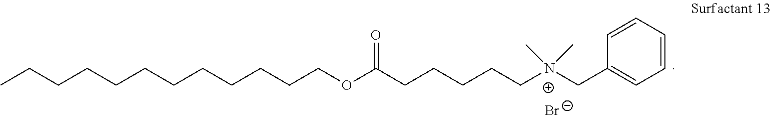 [SMILES: CCCCCCCCCCCCOC(=O)CCCCC[N+](C)(C)Cc1ccccc1 ; [Br-]]

One specific compound provided by the present disclosure and referred to herein as Surfactant 14 is 6-(dodecyloxy)-N-(2-(2-methoxyethoxy)ethyl)-N,N-dimethyl-6-oxohexan-1-aminium bromide; having the formula:

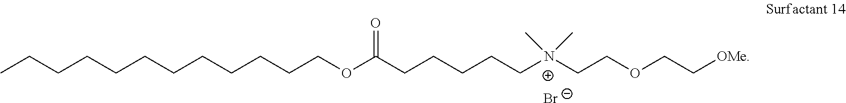 [SMILES: CCCCCCCCCCCCOC(=O)CCCCC[N+](C)(C)CCOCCOC ; [Br-]]

One specific compound provided by the present disclosure and referred to herein as Surfactant 15 is 6-(dodecyloxy)-N,N-dimethyl-6-oxo-N-phenethylhexan-1-aminium iodide; having the formula: Surfactant 15

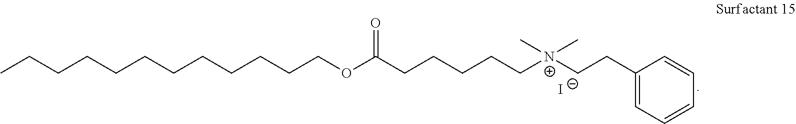 [SMILES: CCCCCCCCCCCCOC(=O)CCCCC[N+](C)(C)CCc1ccccc1 ; [I-]]

One specific compound provided by the present disclosure and referred to herein as Surfactant 16 is N-(2-(dimethylamino)-2-oxoethyl)-6-(dodecyloxy)-N-methyl-6-oxohexan-1-aminium chloride; having the formula:

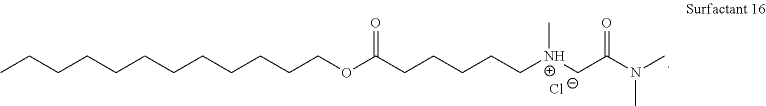 [SMILES: CCCCCCCCCCCCOC(=O)CCCCC[NH+](C)CC(=O)N(C)C ; [Cl-]]

One specific compound provided by the present disclosure and referred to herein as Surfactant 17 is sodium 4-((6-(dodecyloxy)-6-oxohexyl)(methyl)amino)butane-1-sulfonate; having the formula:

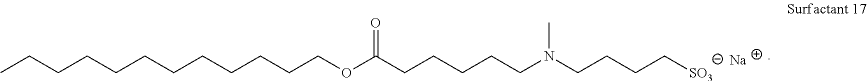

One specific compound provided by the present disclosure and referred to herein as Surfactant 18 is sodium 5-((6-(dodecyloxy)-6-oxohexyl)(methyl)amino)pentanoate; having the formula:

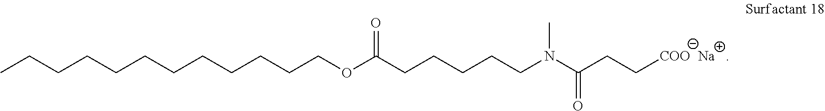

One specific compound provided by the present disclosure and referred to herein as Surfactant 19 is sodium 5-((6-(dodecyloxy)-6-oxohexyl)amino)pentanoate; having the formula:

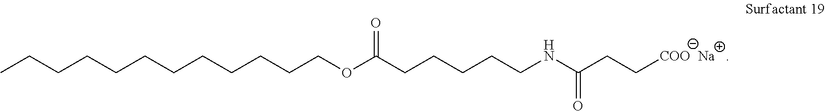

One specific compound provided by the present disclosure and referred to herein as Surfactant 20 is sodium 4-((6-(dodecyloxy)-6-oxohexyl)amino)butane-1-sulfonate; having the formula:

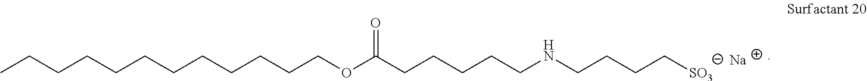

One specific compound provided by the present disclosure and referred to herein as Surfactant 21 is dodecyl 6-(2,3,4,5,6-pentahydroxy-N-methylhexanamido)hexanoate; having the formula:

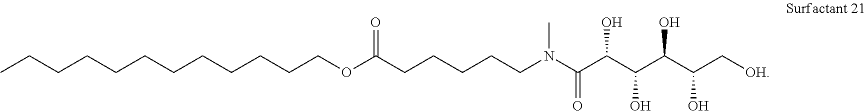 [SMILES: CCCCCCCCCCCCOC(=O)CCCCCN(C)C(=O)[C@H](O)[C@@H](O)[C@@H](O)[C@@H](O)CO]

One specific compound provided by the present disclosure and referred to herein as Surfactant 22 is 1,4-bis(6-(dodecyloxy)-6-oxohexyl)piperazine-1,4-diium dichloride; having the formula:

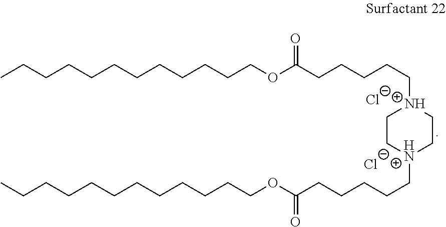

One specific compound provided by the present disclosure and referred to herein as Surfactant 23 is didodecyl 6,6′-((2,3-dihydroxysuccinyl)bis(methylazanediyl))dihexanoate; having the formula:

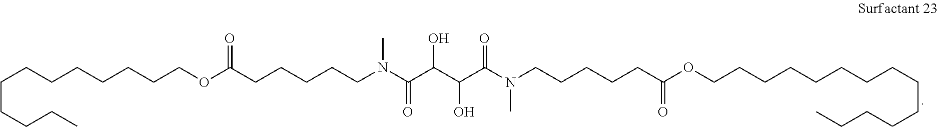 [SMILES: CCCCCCCCCCCCCCOC(=O)CCCCCN(C)C(=O)C(O)C(O)C(=O)N(C)CCCCCC(=O)OCCCCCCCCCCCC]

One specific compound provided by the present disclosure and referred to herein as Surfactant 24 is 1,4-bis(6-(dodecyloxy)-6-oxohexyl)-1,4-dimethylpiperazine-1,4-diium diiodide; having the formula:

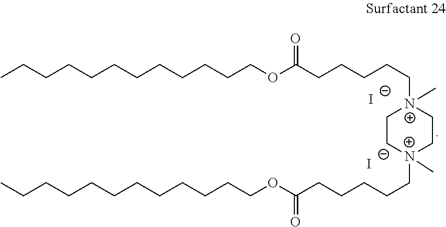

The compounds disclosed herein may be synthesized by various methods. One such method includes opening a lactam to yield an amino acid having an N-terminus and reacting the N-terminus of the amino acid with an alkylating agent to yield a tertiary amine. The resulting tertiary amine may then react with an alcohol under acidic conditions to provide an amino acid ester having an N-terminus. The amino acid ester N-terminus may then react with an acid to yield a quaternary amine salt.

The amino acid may be naturally occurring or synthetic or may be derived from a ring opening reaction of a lactam, such as propiolactam, butyrolactam, valerolactam, and caprolactam, for example. The ring-opening reaction may be either an acid or alkali catalyzed reaction, and an example of an acid catalyzed reaction is shown below in Scheme 1.

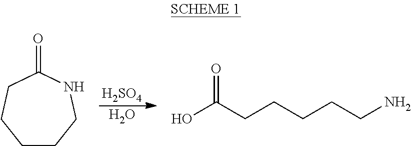

The amino acid may have as few as 2 or as many as 5, namely 2, 3, 4, or 5, carbons between the N- and C-termini. The alkyl chain may be branched or straight. The alkyl chain may be interrupted with nitrogen, oxygen, or sulfur. The alkyl chain may be further substituted with one or more substituents selected from the group consisting of hydroxyl, amino, amido, sulfonyl, sulfonate, carboxyl, and carboxylate. The N-terminal nitrogen may be acylated or alkylated with one or more alkyl groups. For example, the amino acid may be 6-(dimethylamino)hexanoic acid.

The derivative of the amino acid may be synthesized as shown below in Scheme 2. As shown, 6-aminohexanoic acid is treated with formaldehyde in formic acid at reflux to give 6-(dimethylamino)hexanoic acid. The free carboxylic acid is then treated with an alcohol, such as dodecanol, in the presence of p-toluene sulfonic acid (PTSA) in toluene to give the corresponding ester, dodecyl 6-(dimethylamino)hexanoate. The N-terminus is then alkylated with methyl iodide in the presence of sodium carbonate.

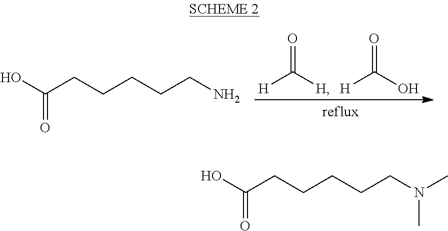

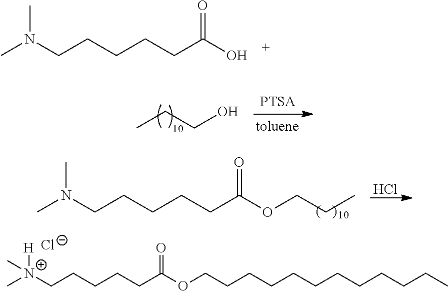

The compounds of the present disclosure are provided for use as surfactants. The present disclosure therefore provides a surfactant of Formula (I) as disclosed above.

The present disclosure further provides the use of a compound of Formula (I) as disclosed above as a surfactant.

The compounds of the present disclosure are surfactants and demonstrate surface-active properties. These properties may be measured and described by various methods. One method by which surfactants may be described is by the molecule's critical micelle concentration (CMC). CMC may be defined as the concentration of a surfactant at which micelles form, and above which all additional surfactant is incorporated into micelles.

The compounds of formula (I) may have a critical micelle concentration (CMC) from about 0.001 mmol to about 10 mmol, or from about 0.01 mmol to about 5 mmol, or from about 0.01 mmol to about 2 mmol or from about 0.01 mmol or about 1.36 mmol, or within any range defined between any two of the foregoing values as endpoints. Critical micelle concentration (CMC) is defined as the surfactant concentration at which micelle formation is first seen in the solution. The surface tension of the solutions at critical micelle concentration (CMC) is defined as γCMC.

As surfactant concentration increases, surface tension decreases. Once the surface is completely overlaid with surfactant molecules, micelles begin to form. This point represents the CMC, as well as the minimum surface tension. Further addition of surfactant will not further affect the surface tension. CMC may therefore be measured by observing the change in surface tension as a function of surfactant concentration. One such method for measuring this value is the Wilhemy plate method. A Wilhelmy plate is usually a thin iridium-platinum plate attached to a balance by a wire and placed perpendicularly to the air-liquid interface. The balance is used to measure the force exerted on the plate by wetting. This value is then used to calculate the surface tension (γ) according to Equation 1:

$$γ = F / I cos θ \qquad Equation 1$$

wherein I is equal to the wetted perimeter (2w+2d, in which w and d are the plate thickness and width, respectively) and cos θ, the contact angle between the liquid and the plate, is assumed to be 0 in the absence of an extant literature value.

Another parameter used to assess the performance of surfactants is dynamic surface tension. The dynamic surface tension is the value of the surface tension for a particular surface or interface age. In the case of liquids with added surfactants, this can differ from the equilibrium value. Immediately after a surface is produced, the surface tension is equal to that of the pure liquid. As described above, surfactants reduce surface tension; therefore, the surface tension drops until an equilibrium value is reached. The time required for equilibrium to be reached depends on the diffusion rate and the adsorption rate of the surfactant.

A plateau value of minimum surface tension that can be reached the compounds of formula (I) may be from about 10 mN/m to about 40 mN/m, or about 20 mN/m to about 25 mN/m, or from about 20 mN/m to about 23 mN/mm or within any range defined between any two of the foregoing values as endpoints.

One method by which dynamic surface tension is measured relies upon a bubble pressure tensiometer. This device measures the maximum internal pressure of a gas bubble that is formed in a liquid by means of a capillary. The measured value corresponds to the surface tension at a certain surface age, the time from the start of the bubble formation to the occurrence of the pressure maximum. The dependence of surface tension on surface age can be measured by varying the speed at which bubbles are produced.

Surface-active compounds may also be assessed by their wetting ability on solid substrates as measured by the contact angle. When a liquid droplet comes in contact with a solid surface in a third medium, such as air, a three-phase line forms among the liquid, the gas and the solid. The angle between the surface tension unit vector, acting at the three-phase line and tangent at the liquid droplet, and the surface is described as the contact angle. The contact angle (also known as wetting angle) is a measure of the wettability of a solid by a liquid. In the case of complete wetting, the liquid is completely spread over the solid and the contact angle is 0°. Wetting properties are typically measured for a given compound at the concentration of 1-10× CMC however, it is not a property that is concentration-dependent. Therefore, measurements of wetting properties can be measured at concentrations that are higher or lower.

In one method, an optical contact angle goniometer may be used to measure the contact angle. This device uses a digital camera and software to extract the contact angle by analyzing the contour shape of a sessile droplet of liquid on a surface.

The identity surface-active compounds of the instant disclosure may be analyzed and confirmed using nuclear magnetic resonance (NMR) spectroscopy, performed on a Bruker 500 MHz spectrometer. The critical micelle concentration (CMC) may be determined by the Wilhelmy plate method at 23° C. with a tensiometer (DCAT 11, DataPhysics Instruments GmbH) equipped with a Pt-Ir plate. Dynamic surface tension may be determined with a bubble pressure tensiometer (Kruss BP100, Kruss GmbH), at 23° C. Contact angle may be determined with the optical contact angle goniometer (OCA 15 Pro, DataPhysics GmbH) equipped with a digital camera. The pH was determined using a standard laboratory pH meter.

The synthesized molecules may be further characterized for their thermodynamic behavior at the phase interface, for example using pre-factors determined from Gibbs Thermodynamics equations (shown here) measuring the thermodynamics of the adsorption of a surfactant molecule at a 2-phase interface, typically an air and liquid interface.

$${Γ}_{S} = - \frac{1}{2 RT} {( \frac{\partial γ}{\partial ln C} )}_{T , p} ,$$

where R is the gas constant, T is the temperature, C the concentration in the bulk phase.

As a general matter, ionic surfactants would necessarily incur the presence of a cation and an ion as they disassociate at the interface, whereas a non-ionic surfactant would have just the molecule itself as it dissociates. Pre-factors are used to refer to the surface excess concentration of a substance as it thermodynamically disassociates at a 2-phase interface. Accordingly, an ionic surfactant has a pre-factor (or prefactor) of two (2), which accounts for the two species being cation and the anion as the ionic solution disassociates in water, whereas a nonionic surfactant has a prefactor of one (1).

Potential applications for the surface-active compounds of the present disclosure include, without limitation, formulations for use as personal care products, shampoos, hair conditioners, body cleansers, mascaras, toothpaste, detergents, cleaning products, including cleaning products used in food processing places, water based cleaning formulations, dry cleaning, foaming agents, emulsifiers, degreasers, spot-free rinsing solutions, spot-free drying solutions, floor and carpet cleaners, cleaning agents for graffiti removal, agricultural products, pesticide and plant growth regulator formulations, fungicides, herbicides, insecticides, adjuvants, spray drift reducing agents, wetting agents for crop protection, adjuvants for crop protection, wetting agents for aerosol spray coatings, inks, paints, adhesives, paint strippers, healthcare products, solid dosage formulations, liquid dosage formulations, intravenous perfusions, prescription drugs, over the counter drugs, minerals, herbals, vitamin supplements, drugs administered in hospitals or other places of palliative care, vaccines, tissue transplants, grafts or infusions, organ transplants, grafts or infusions, cell transplants, grafts or infusions, wound care, topical ointments, lotions, cleaners, wipes, bandages, dressings, oil and gas production products, fracking fluids, improved oil recovery formulations, fluids for recovering bio-based oils, hydrocarbon recovery, electronics, pre-texturing agents, etchants, and photoresist strippers. Moreover, any of the preceding recited formulations may include one or more additional components, such as co-surfactants, foaming agents, foam boosters, thickeners, rheology modifiers, pH stabilizers, fragrances, emulsifiers, salts, hydrotropes, foaming/detersive agents, solubilizing agents, coloring agents, propellants, pigmentations, dispersant agents, solvents/carriers, and anti-foaming agents.

It will be understood by one skilled in the art that small differences between compounds may lead to substantially different surfactant properties, such that different compounds may be used with different substrates, in different applications. It will be further understood by one skilled in the art that surfactant properties may not be predictable on the basis of chemical structure, as further demonstrated below.

The following is provided to demonstrate the different properties of the different surfactants. In Table 1 below, short names for the surfactants are correlated with their corresponding chemical structures.

TABLE 1

| Surfactant | Formula & Name |
|---|---|
| Surfactant 1 C₂₃H₄₀BrNO₂ M.Wt: 442.4820 CMC: 0.825 mM | 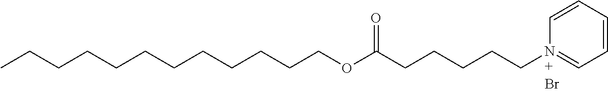 |
| | 1-(6-(dodecyloxy)-6-oxohexyl)pyridin-1-ium bromide |
| Surfactant 2 C₂₅H₅₀INO₄ M.Wt: 555.5825 CMC: 0.9176 mM | 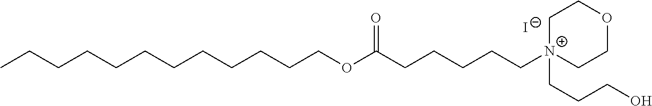 [SMILES: CCCCCCCCCCCCOC(=O)CCCCC[N+]1(CCCO)CCOCC1 ; [I-]] |
| | 4-(6-(dodecyloxy)-6-oxohexyl)-4-(3- |
| | hydroxypropyl)morpholin-4-ium iodide |
| Surfactant 3 C₂₃H₄₆ClNO₂ M.Wt: 404.0760 CMC: 0.1519 mM | 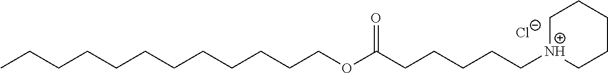 [SMILES: CCCCCCCCCCCCOC(=O)CCCCC[NH+]1CCCCC1 ; [Cl-]] |
| | 1-(6-(dodecyloxy)-6-oxohexyl)piperidin-1-ium chloride |
| Surfactant 4 C₂₂H₄₄ClNO₃ M.Wt: 406.0480 CMC: 0.1089 mM | 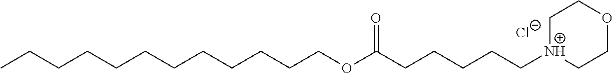 [SMILES: CCCCCCCCCCCCOC(=O)CCCCC[NH+]1CCOCC1 ; [Cl-]] |
| | 4-(6-(dodecyloxy)-6-oxohexyl)morpholin-4-ium chloride |
| Surfactant 5 C₂₃H₄₈Cl₂N₂O₂ M.Wt: 455.5490 CMC: 0.2105 mM | 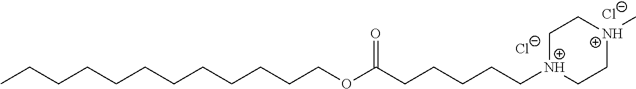 [SMILES: [Cl-] ; CCCCCCCCCCCCOC(=O)CCCCC[NH+]1CC[NH+](C)CC1 ; [Cl-]] |
| | 1-(6-(dodecyloxy)-6-oxohexyl)-4-methylpiperazine-1,4- |
| | diium dichloride |
| Surfactant 6 C₂₂H₄₃NO₄ M.Wt: 385.5890 CMC: 0.0293 mM | 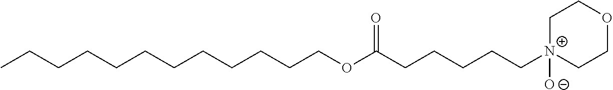 [SMILES: CCCCCCCCCCCCOC(=O)CCCCC[N+]1([O-])CCOCC1] |
| | 4-(6-(dodecyloxy)-6-oxohexyl)morpholine 4-oxide |
| Surfactant 7 C₂₃H₄₆INO₂ M.Wt: 495.5305 CMC: 0.7886 mM | 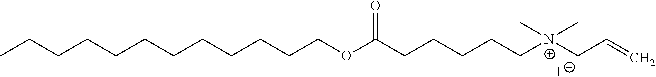 [SMILES: C=CC[N+](C)(C)CCCCCC(=O)OCCCCCCCCCCCC ; [I-]] |
| | N-allyl-6-(dodecyloxy)-N,N-dimethyl-6-oxohexan-1- |
| | aminium iodide |
| Surfactant 8 C₂₂H₄₆INO₃ M.Wt: 499.5185 CMC: 0.4109 mM | 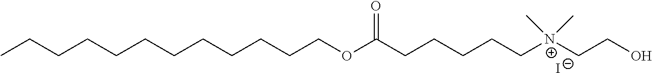 [SMILES: CCCCCCCCCCCCOC(=O)CCCCC[N+](C)(C)CCO ; [I-]] |
| | 6-(dodecyloxy)-N-(2-hydroxyethyl)-N,N-dimethyl-6- |
| | oxohexan-1-aminium iodide |
| Surfactant 9 C₂₄H₄₈BrNO₄ M.Wt: 494.5550 CMC: 0.0662 mM | 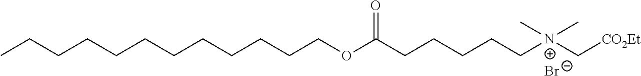 [SMILES: CCCCCCCCCCCCOC(=O)CCCCC[N+](C)(C)CC(=O)OCC ; [Br-]] |
| | 6-(dodecyloxy)-N-(2-ethoxy-2-oxoethyl)-N,N-dimethyl-6- |
| | oxohexan-1-aminium bromide |
| Surfactant 10 C₂₃H₄₄BrNO₂ M.Wt: 446.5140 CMC: 0.2463 mM | 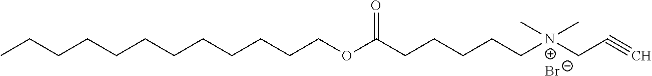 [SMILES: C#CC[N+](C)(C)CCCCCC(=O)OCCCCCCCCCCCC ; [Br-]] |
| | 6-(dodecyloxy)-N,N-dimethyl-6-oxo-N-(prop-2-yn-1- |
| | yl)hexan-1-aminium bromide |
| Surfactant 11 C₂₃H₄₈INO₃ M.Wt: 513.5455 CMC: 0.1771 mM | 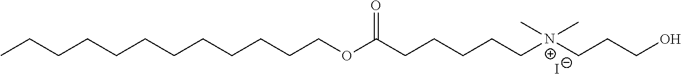 [SMILES: CCCCCCCCCCCCOC(=O)CCCCC[N+](C)(C)CCCO ; [I-]] |
| | 6-(dodecyloxy)-N-(3-hydroxypropyl)-N,N-dimethyl-6- |
| | oxohexan-1-aminium iodide |
| Surfactant 12 C₂₄H₄₉IN₂O₃ M.Wt: 540.5715 CMC: 0.1573 mM | 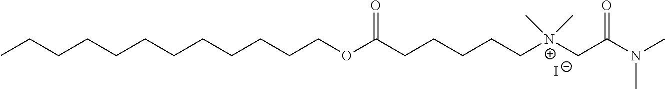 [SMILES: CCCCCCCCCCCCOC(=O)CCCCC[N+](C)(C)CC(=O)N(C)C ; [I-]] |
| | N-(2-(dimethylamino)-2-oxoethyl)-6-(dodecyloxy)-N,N- |
| | dimethyl-6-oxohexan-1-aminium iodide |
| Surfactant 13 C₂₇H₄₈BrNO₂ M.Wt: 498.5900 CMC: 0.1487 mM | 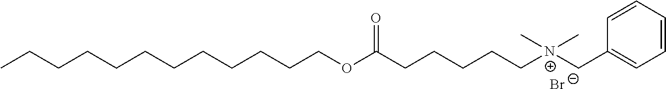 [SMILES: CCCCCCCCCCCCOC(=O)CCCCC[N+](C)(C)Cc1ccccc1 ; [Br-]] |
| | N-benzyl-6-(dodecyloxy)-N,N-dimethyl-6-oxohexan-1- |
| | aminium bromide |
| Surfactant 14 C₂₅H₅₂BrNO₄ M.Wt: 510.5980 CMC: 0.2940 mM | 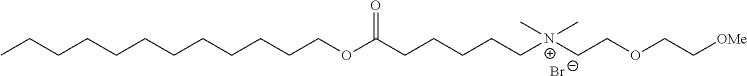 [SMILES: CCCCCCCCCCCCOC(=O)CCCCC[N+](C)(C)CCOCCOC ; [Br-]] |
| | 6-(dodecyloxy)-N-(2-(2-methoxyethoxy)ethyl)-N,N- |
| | dimethyl-6-oxohexan-1-aminium bromide |
| Surfactant 15 C₂₈H₅₀INO₂ M.Wt: 559.6175 (Low solubility in water at room temperature, CMC measurements must be taken when heated) | 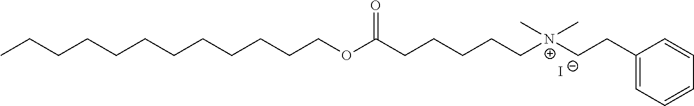 [SMILES: CCCCCCCCCCCCOC(=O)CCCCC[N+](C)(C)CCc1ccccc1 ; [I-]] |
| | 6-(dodecyloxy)-N,N-dimethyl-6-oxo-N-phenethylhexan- |
| | 1-aminium iodide |
| Surfactant 16 C₂₃H₄₇ClN₂O₃ M.Wt: 435.0900 CMC: 0.2379 mM | 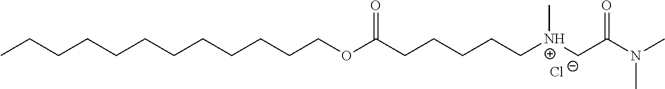 [SMILES: CCCCCCCCCCCCOC(=O)CCCCC[NH+](C)CC(=O)N(C)C ; [Cl-]] |
| | N-(2-(dimethylamino)-2-oxoethyl)-6-(dodecyloxy)-N- |
| | methyl-6-oxohexan-1-aminium chloride |
| Surfactant 17 C₂₃H₄₆NNaO₅S M.Wt: 471.6728 CMC: 0.0260 mM | 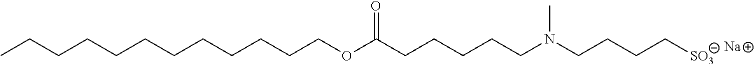 |
| | sodium 4-((6-(dodecyloxy)-6- |
| | oxohexyl)(methyl)amino)butane-1-sulfonate |
| Surfactant 18 C₂₃H₄₂NNaO₅ M.Wt: 435.5808 CMC: 0.1461 mM | 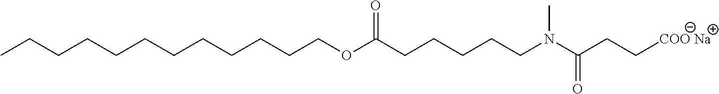 |
| | sodium 5-((6-(dodecyloxy)-6- |
| | oxohexyl)(methyl)amino)pentanoate |
| Surfactant 19 C₂₂H₄₀NNaO₅ M.Wt: 421.5538 CMC: 0.2670 mM | 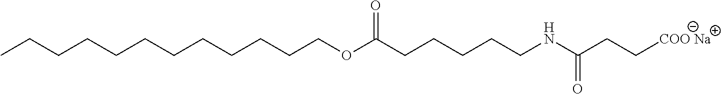 |
| | sodium 5-((6-(dodecyloxy)-6- |
| | oxohexyl)amino)pentanoate |
| Surfactant 20 C₂₂H₄₄NNaO₅S M.Wt: 457.6458 CMC: 0.4455 mM | 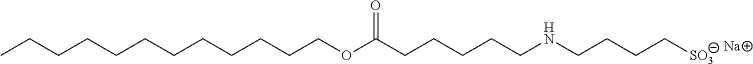 |
| | sodium 4-((6-(dodecyloxy)-6-oxohexyl)amino)butane-1- |
| | sulfonate |
| Surfactant 21 C₂₅H₄₉NO₈ M.Wt: 491.6660 CMC: 0.0132 mM | 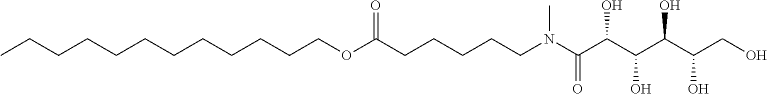 [SMILES: CCCCCCCCCCCCOC(=O)CCCCCN(C)C(=O)[C@H](O)[C@@H](O)[C@@H](O)[C@@H](O)CO] |
| | dodecyl 6-(2,3,4,5,6-pentahydroxy-N- |
| | methylhexanamido)hexanoate |
| Surfactant 22 C₄₀H₈₀Cl₂N₂O₄ M.Wt: 723.9900 CMC: 0.3398 mM | 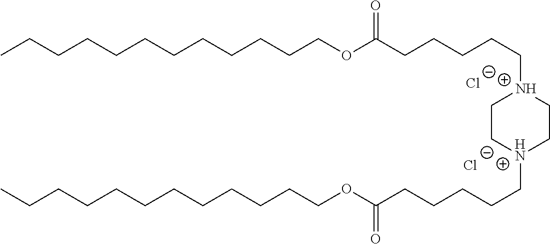 |
| | 1,4-bis(6-(dodecyloxy)-6-oxohexyl)piperazine-1,4-diium dichloride |
| Surfactant 23 C₄₂H₈₀N₂O₈ M.Wt: 741.1080 CMC: 0.0404 mM | 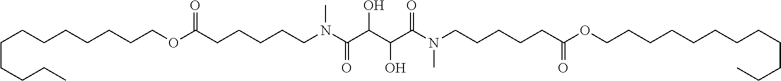 [SMILES: CCCCCCCCCCCCOC(=O)CCCCCN(C)C(=O)C(O)C(O)C(=O)N(C)CCCCCC(=O)OCCCCCCCCCCCC] |
| | didodecyl 6,6′-((2,3- |
| | dihydroxysuccinyl)bis(methylazanediyl))dihexanoate |
| Surfactant 24 C₄₂H₈₄I₂N₂O₄ M.Wt: 934.9529 CMC: 0.9089 mM | 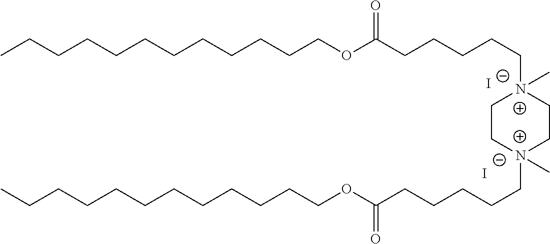 [SMILES: CCCCCCCCCCCCOC(=O)CCCCC[N+]1(C)CC[N+](C)(CCCCCC(=O)OCCCCCCCCCCCC)CC1 ; [I-] ; [I-]] |
| | 1,4-bis(6-(dodecyloxy)-6-oxohexyl)-1,4- |
| | dimethylpiperazine-1,4-diium diiodide |

These compounds may be effective as surfactants, useful for wetting or foaming agents, dispersants, emulsifiers, and detergents, among other applications.

The present disclosure further provides a formulation comprising a compound as disclosed herein. The amount of the compounds disclosed herein used in a formulation may be as low as about 0.001 wt. %, about 0.05 wt. %, about 0.1 wt. %, about 0.5 wt. %, about 1 wt. %, about 2 wt. %, or about 5 wt. %, or as high as about 8 wt. %, about 10 wt. %, about 15 wt. %, about 20 wt. %, or about 25 wt. %, or within any range defined between any two of the foregoing values.

### Examples

### Example 1: Synthesis of Surfactant 1

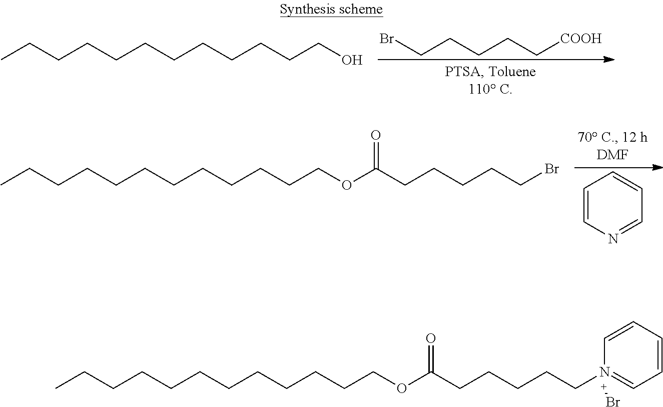

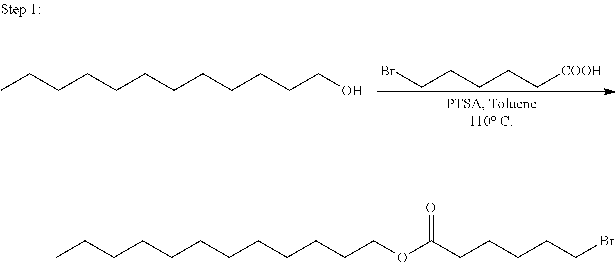 [SMILES: CCCCCCCCCCCCO ; O=C(O)CCCCCBr ; CCCCCCCCCCCCOC(=O)CCCCCBr]

Step 1 synthesis procedure: Bromo hexanoic acid (1 equiv) followed by PTSA (1 equiv) were added to 1 g of dodecanol (1 equiv) in 20 mL of toluene at room temperature and then heated at 110° C. for 14 h. The reaction mixture was then diluted with DCM and washed with a Saturated (or “Sat.”) NaHCO₃ solution (50 mL) followed by water (50 mL). The organic layer concentrated, and crude compound used for next step without any further purification. 1.8 g of yellow color liquid was obtained. (Giving a yield of about 90%).

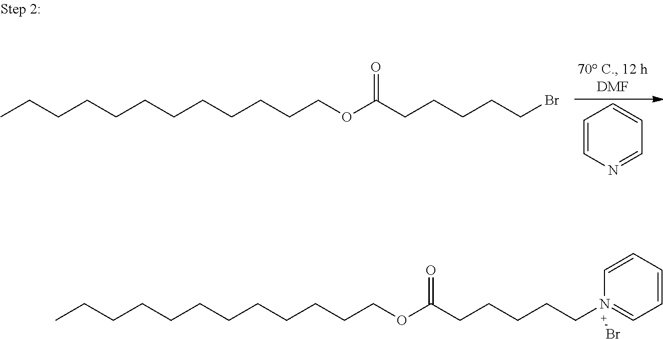

Step 2 synthesis procedure: Pyridine (1 equiv) was added to 1 g of bromo hexanoic ester (1 equiv) in 20 mL of DMF at room temperature and then heated at 70° C. for 14 h. The reaction mixture was then concentrated, and crude compound was washed with hexanes to remove unreacted starting materials. 1.2 g of light brown liquid was obtained. (Giving a yield of about 95%).

### Example 1a: Determination of Physical Properties of Surfactant 1

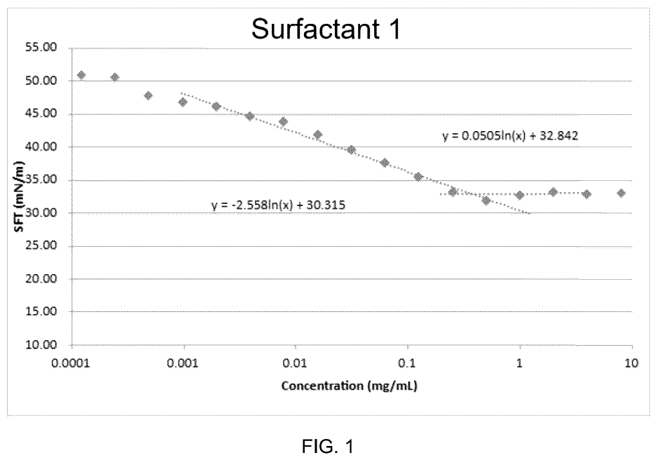

The critical micelle concentration (CMC) for Surfactant 1 was measured. From the surface tension change with concentration in water, the CMC was determined to be about 0.8250 mM (0.3650 mg/mL) at pH 4. The plateau value of minimum surface tension that can be reached by this surfactant was about 32.88 mN/m, indicating that the surfactant has outstanding interfacial activity. These results were plotted as surface tension versus concentration in FIG. 1.

The molecular parameters of surfactant 1 are summarized in Table 2:

TABLE 2

| | |
|---|---|
| CMC (mg/mL) | 0.3650 |
| CMC (mM) | 0.8250 |
| γ_{cmc} (mN/m) | 32.88 |
| 10³ × Γₘₐₓ (mmol/m²) | 0.52 |
| Åₘᵢₙ (Å²) | 316.44 |
| pH @ 0.005 mg/mL | 5.71 |
| pH @ 8 mg/mL | 4.05 |
| prefactor | 2 |

### Example 2: Synthesis of Surfactant 2

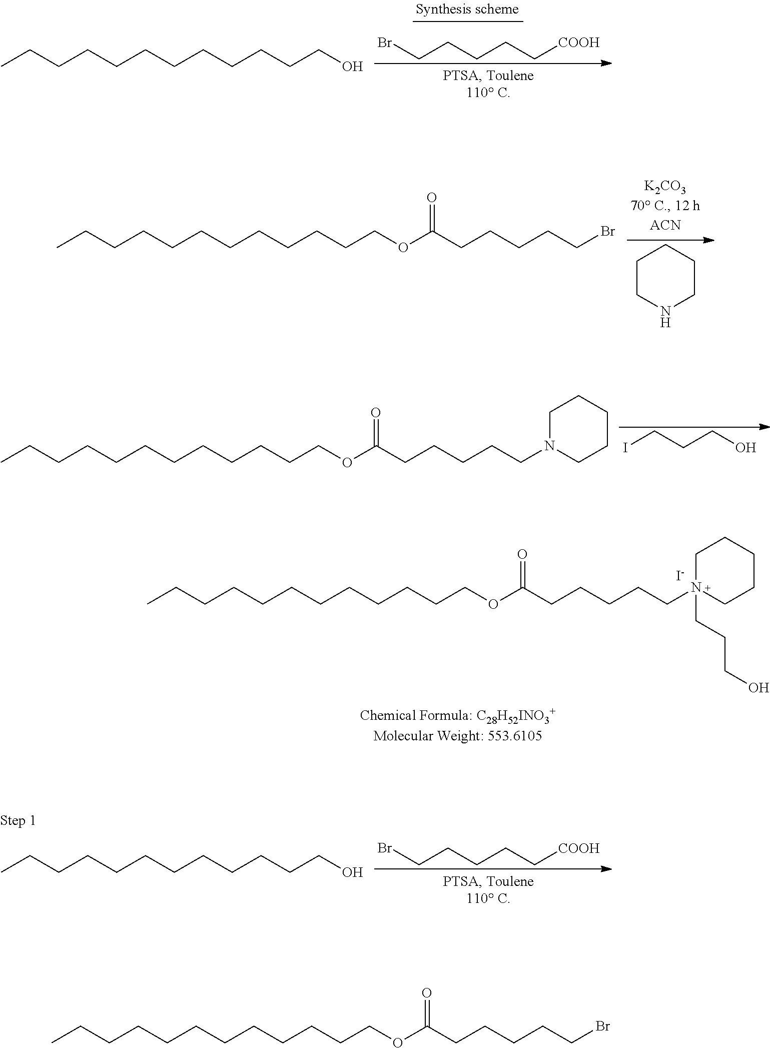

Step 1 synthesis procedure: Bromo hexanoic acid (1 equiv) followed by PTSA (1 equiv) were added to 1 g of dodecanol (1 equiv) in 20 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 1.8 g of yellow color liquid was obtained. (Giving a yield of about 90%).

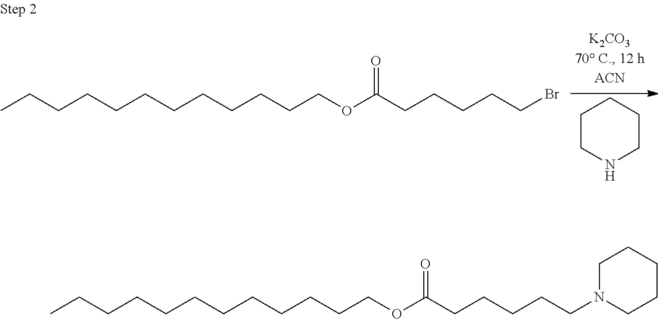

Step 2 synthesis procedure: Piperidine (1 equiv) followed by K₂CO₃ (1 equiv) were added to 1 g of bromo hexanoic ester (1 equiv) in 20 mL of ACN at room temperature and then heated at 70° C. for 12 h. The reaction mixture was filtered, then filtrate was concentrated, further crude compound was washed with hexanes to remove unreacted starting materials. 1 g of light brown liquid was obtained. (giving a yield of about 98%).

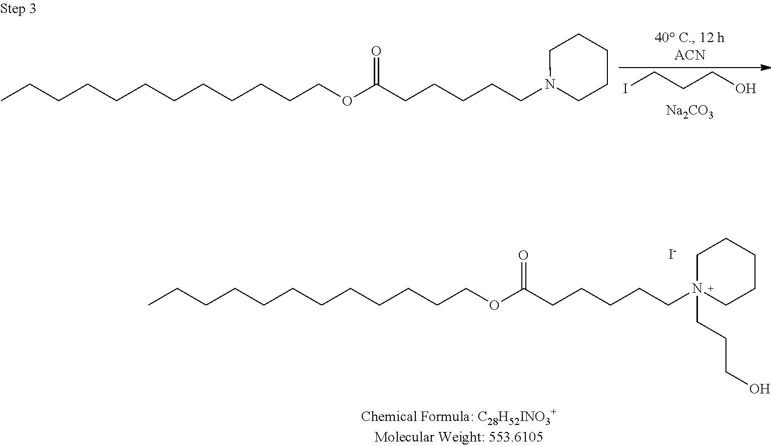

Step 3 synthesis procedure: Iodo propanol (1 equiv) followed by Na₂CO₃ (1 equiv) were added to 1 g of piperidine hexanoic ester (1 equiv) in 20 mL of ACN at room temperature and then heated at 40° C. for 12 h. The reaction mixture was filtered, then filtrate was concentrated, further crude compound was washed with hexanes to remove unreacted starting materials. 1.1 g of light-yellow liquid was obtained. (Giving a yield of about 73%).

### Example 2a: Determination of Physical Properties of Surfactant 2

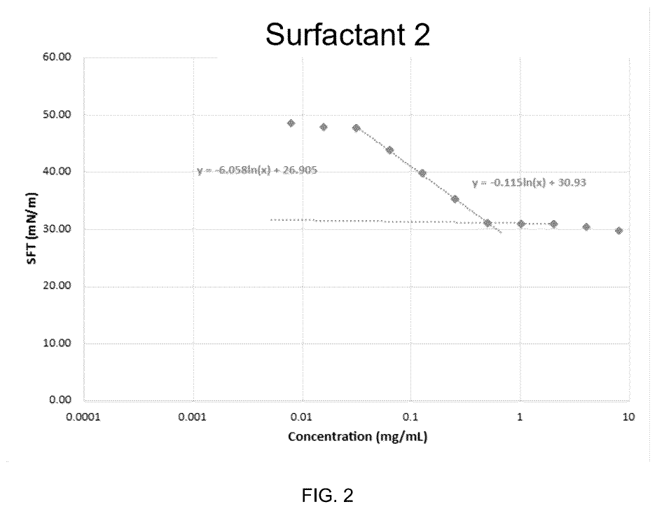

The critical micelle concentration (CMC) for Surfactant 2 was measured. From the surface tension change with concentration in water, the CMC was determined to be about 0.9176 mM (0.5080 mg/mL) at pH 5. The plateau value of minimum surface tension that can be reached by this surfactant was about 30.66 mN/m, indicating that the surfactant has outstanding interfacial activity. These results were plotted as surface tension versus concentration in FIG. 2.

The molecular parameters of surfactant 2 are summarized in Table 3:

TABLE 3

| | |
|---|---|
| CMC (mg/mL) | 0.5080 |
| CMC (mM) | 0.9176 |
| γ_{cmc} (mN/m) | 30.66 |
| 10³ × Γₘₐₓ (mmol/m²) | 1.24 |
| Åₘᵢₙ (Å²) | 133.62 |
| pH @ 8 mg/mL | 5.72 |
| pH @ 0.00024 mg/mL | 6.96 |
| prefactor? | 2 |

### Example 3: Synthesis of Surfactant 3

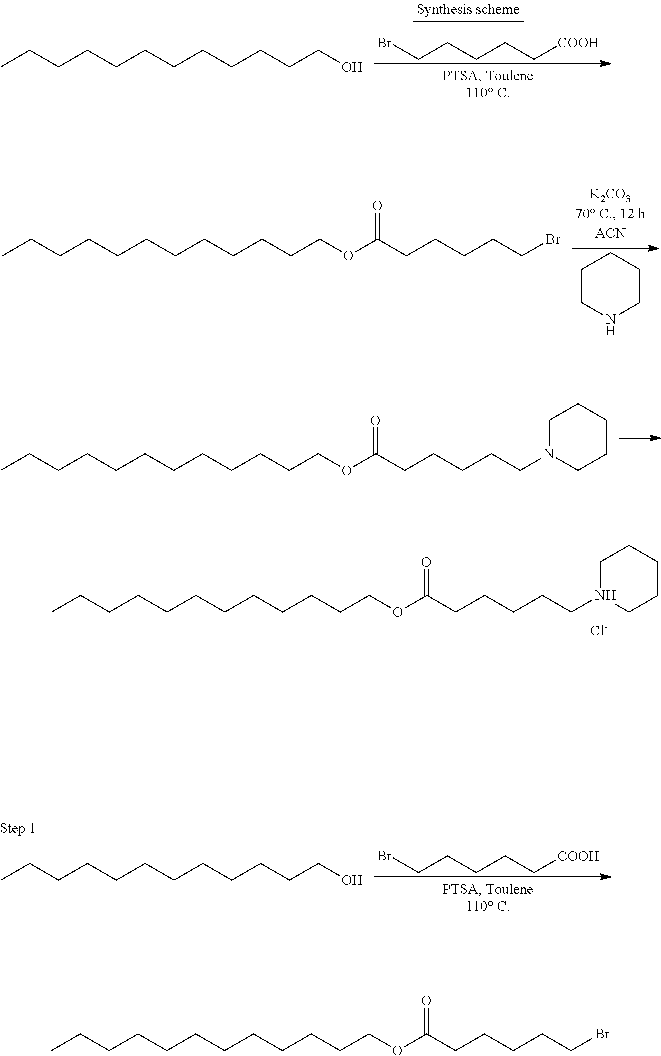

Step 1 synthesis procedure: Bromo hexanoic acid (1 equiv) followed by PTSA (1 equiv) were added to 1 g of dodecanol (1 equiv) in 20 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 1.8 g of yellow color liquid was obtained. (Giving a yield of about 90%).

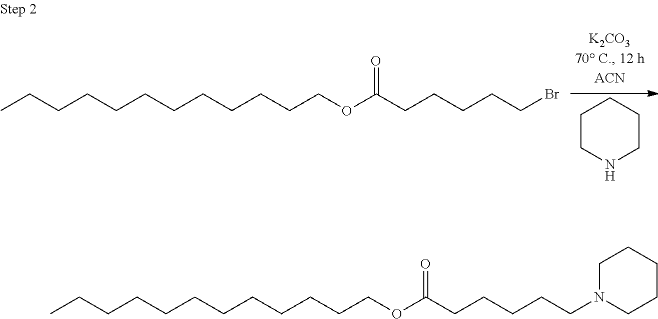

Step 2 synthesis procedure: Piperidine (1 equiv) followed by K₂CO₃ (1 equiv) were added to 1 g of bromo hexanoic ester (1 equiv) in 20 mL of ACN at room temperature and then heated at 70° C. for 12 h. The reaction mixture was filtered, then filtrate was concentrated, further crude compound was washed with hexanes to remove unreacted starting materials. 1 g of light brown liquid was obtained. (Giving a yield of about 98%).

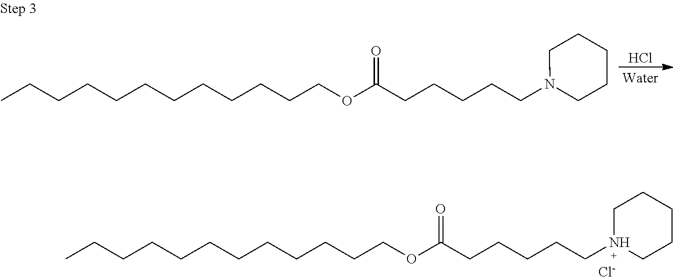 [SMILES: CCCCCCCCCCCCOC(=O)CCCCCN1CCCCC1 ; Cl ; CCCCCCCCCCCCOC(=O)CCCCCN1CCCCC1 ; [Cl-]]

Step 3 synthesis procedure: HCl (1 equiv) was added to 1 g of piperidine hexanoic ester (1 equiv) in 10 mL of water at room temperature and then stirred for 3 h. The reaction mixture was concentrated, further crude compound was washed with hexanes to remove unreacted starting materials. 0.65 g of brown liquid was obtained. (giving a yield of about 55%).

### Example 3a: Determination of Physical Properties of Surfactant 3

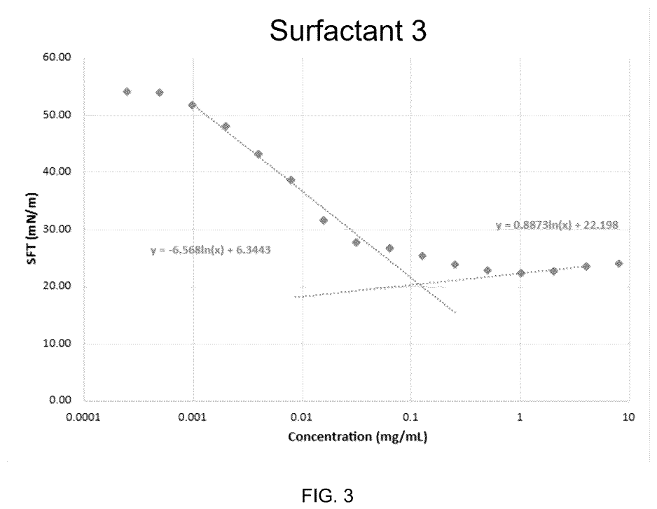

The critical micelle concentration (CMC) for Surfactant 3 was measured. From the surface tension change with concentration in water, the CMC was determined to be about 0.1519 mM (0.0614 mg/mL) at pH 3. The plateau value of minimum surface tension that can be reached by this surfactant was about 22.81 mN/m, indicating that the surfactant has outstanding interfacial activity. These results were plotted as surface tension versus concentration in FIG. 3.

The molecular parameters of surfactant 3 are summarized in Table 4:

TABLE 4

| | |
|---|---|
| CMC (mg/mL) | 0.0614 |
| CMC (mM) | 0.1519 |
| γ_{cmc} (mN/m) | 22.81 |
| 10³ × Γₘₐₓ (mmol/m²) | 1.35 |
| Åₘᵢₙ (Å²) | 123.24 |
| pH @ 8 mg/mL | 2.94 |
| pH @ 0.00024 mg/mL | 5.90 |
| prefactor? | 2 |

### Example 4: Synthesis of Surfactant 4

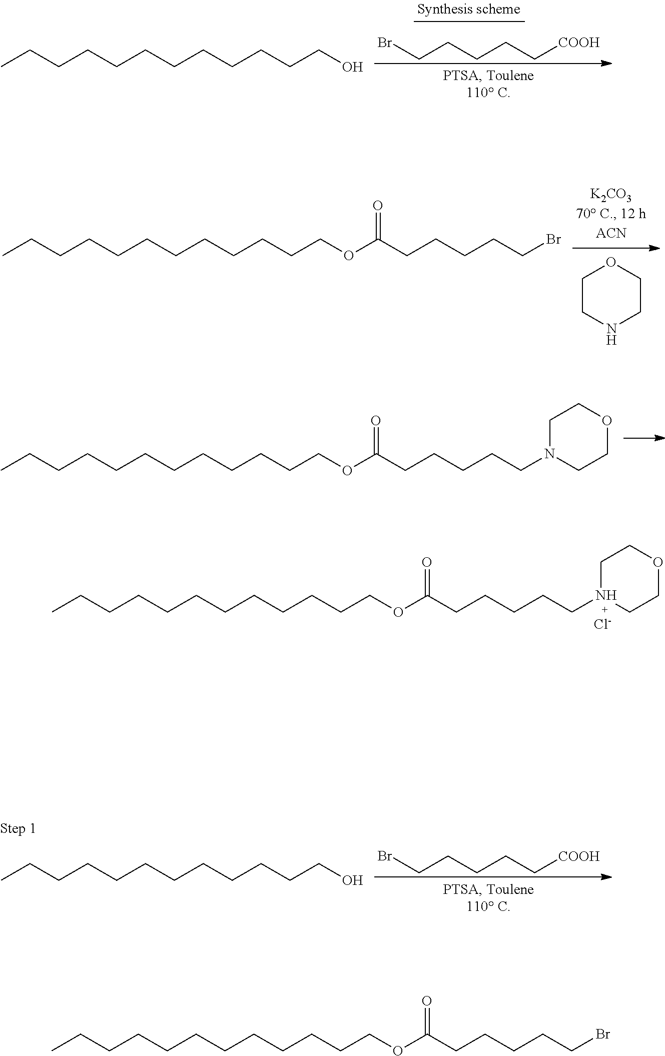

Step 1 synthesis procedure: Bromo hexanoic acid 1 (equiv) followed by PTSA (1 equiv) were added to 1 g of dodecanol (1 equiv) in 20 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 1.8 g of yellow color liquid was obtained. (Giving a yield of about 90%).

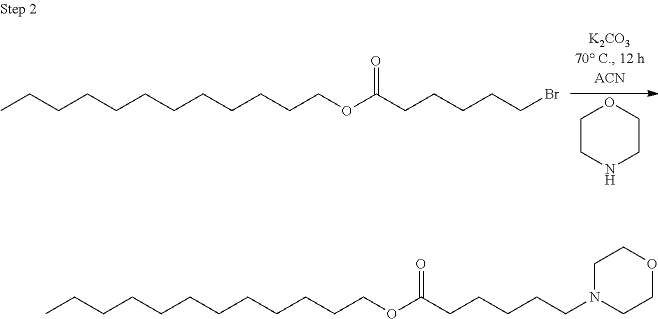

Step 2 synthesis procedure: Morpholine (1 equiv) followed by K₂CO₃ (1 equiv) were added to 1 g of bromo hexanoic ester (1 equiv) in 20 mL of ACN at room temperature and then heated at 70° C. for 12 h. The reaction mixture was filtered, then filtrate was concentrated, further crude compound was washed with hexanes to remove unreacted starting materials. 1 g of light brown liquid was obtained. (Giving a yield of about 98%).

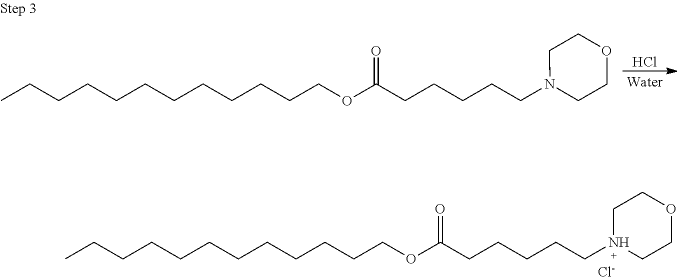 [SMILES: CCCCCCCCCCCCOC(=O)CCCCCN1CCOCC1 ; Cl ; CCCCCCCCCCCCOC(=O)CCCCCN1CCOCC1 ; [Cl-]]

Step 3 synthesis procedure: HCl (1 equiv) was added to 1 g of morpholine hexanoic ester (1 equiv) in 10 mL of water at room temperature and then stirred for 3 h. The reaction mixture was concentrated, further crude compound was washed with hexanes to remove unreacted starting materials. 0.6 g of light-yellow liquid was obtained. (giving a yield of about 56%).

### Example 4a: Determination of Physical Properties of Surfactant 4

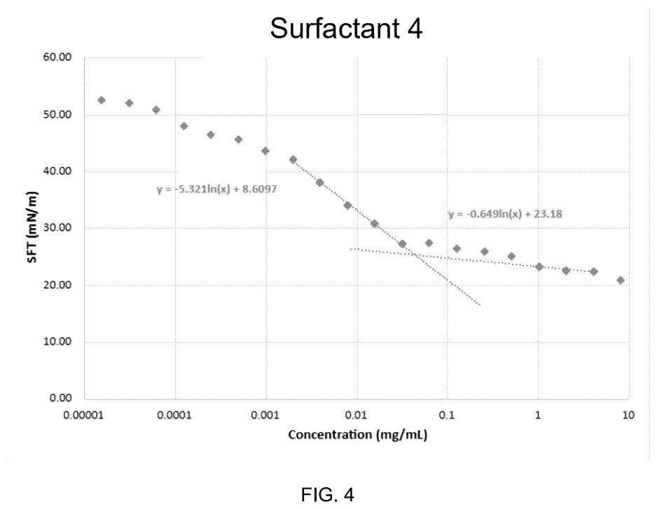

The critical micelle concentration (CMC) for Surfactant 4 was measured. From the surface tension change with concentration in water, the CMC was determined to be about 0.1089 mM (0.0442 mg/mL) at pH 3. The plateau value of minimum surface tension that can be reached by this surfactant was about 22.73 mN/m, indicating that the surfactant has outstanding interfacial activity. These results were plotted as surface tension versus concentration in FIG. 4.

The molecular parameters of surfactant 4 are summarized in Table 5:

TABLE 5

| | |
|---|---|
| CMC (mg/mL) | 0.0442 |
| CMC (mM) | 0.1089 |
| γ_{cmc} (mN/m) | 22.73 |
| 10³ × Γₘₐₓ (mmol/m²) | 2.80 |
| Åₘᵢₙ (Å²) | 59.21 |
| pH @ 8 mg/mL | 2.82 |
| pH @ 0.00002 mg/mL | 5.99 |
| prefactor? | 2 |

### Example 5: Synthesis of Surfactant 5

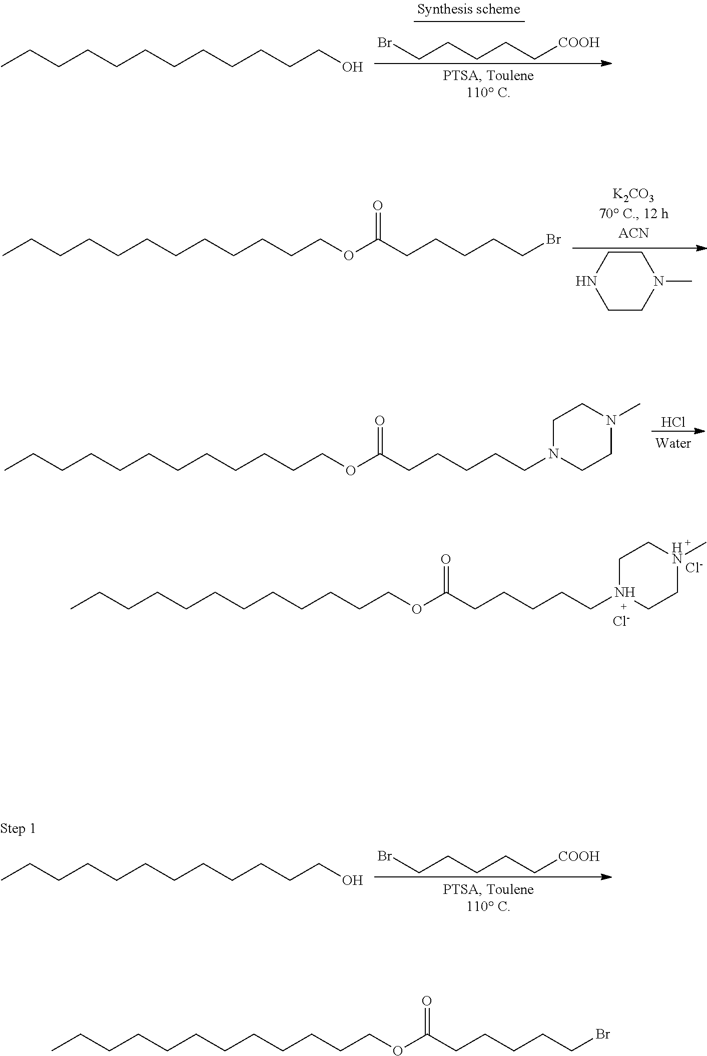

Step 1 synthesis procedure: Bromo hexanoic acid 1 (equiv) followed by PTSA 1 (equiv) were added to 1 g of dodecanol (1 equiv) in 20 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 1.8 g of yellow color liquid was obtained. (Giving a yield of about 90%).

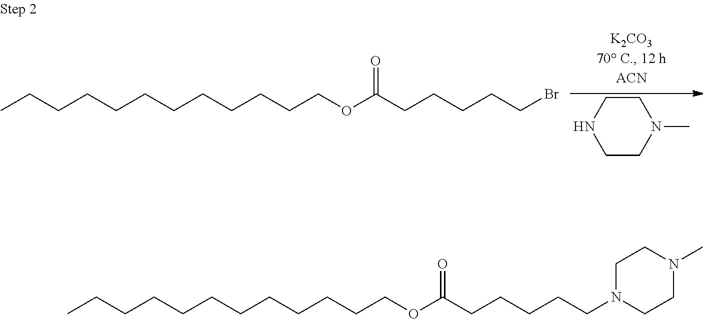

Step 2 synthesis procedure: N-Methyl piperidine (1 equiv) followed by K₂CO₃ (1 equiv) were added to 1 g of bromo hexanoic ester (1 equiv) in 20 mL of ACN at room temperature and then heated at 70° C. for 12 h. The reaction mixture was filtered, then filtrate was concentrated, further crude compound was washed with hexanes to remove unreacted starting materials. 0.95 g of light brown liquid was obtained. (Giving a yield of about 97%).

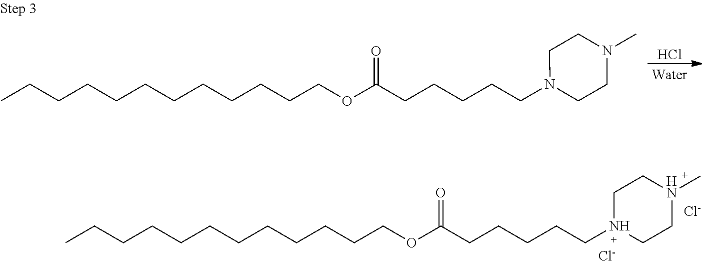

Step 3 synthesis procedure: HCl (2 equiv) was added to 1 g of N-methyl piperazine hexanoic ester (1 equiv) in 10 mL of water at room temperature and then stirred for 3 h. The reaction mixture was concentrated, further crude compound was washed with hexanes to remove unreacted starting materials. 0.7 g of brown liquid was obtained. (giving a yield of about 58%).

### Example 5a: Determination of Physical Properties of Surfactant 5

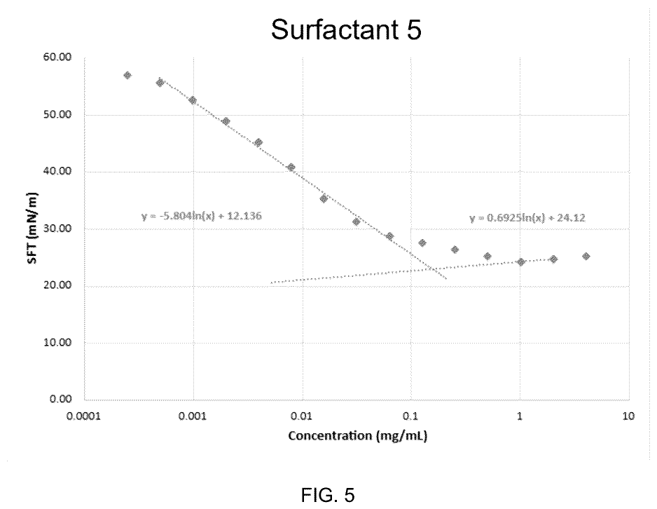

The critical micelle concentration (CMC) for Surfactant 5 was measured. From the surface tension change with concentration in water, the CMC was determined to be about 0.2105 mM (0.0959 mg/mL) at pH 3. The plateau value of minimum surface tension that can be reached by this surfactant was about 24.36 mN/m, indicating that the surfactant has outstanding interfacial activity. These results were plotted as surface tension versus concentration in FIG. 5.

The molecular parameters of surfactant 5 are summarized in Table 6:

TABLE 6

| | |
|---|---|
| CMC (mg/mL) | 0.0959 |
| CMC (mM) | 0.2105 |
| γ_{cmc} (mN/m) | 24.36 |
| 10³ × Γₘₐₓ (mmol/m²) | 0.60 |
| Åₘᵢₙ (Å²) | 278.93 |
| pH @ 8 mg/mL | 2.64 |
| pH @ 0.00024 mg/mL | 5.81 |
| prefactor? | 4 |

### Example 6: Synthesis of Surfactant 6

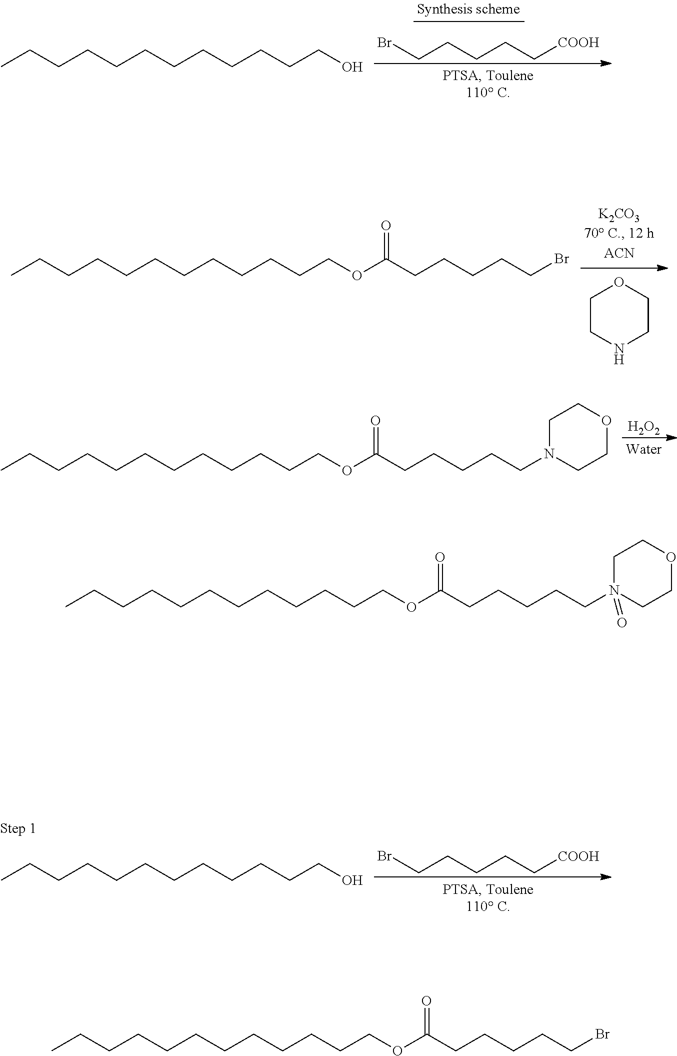

Step 1 synthesis procedure: Bromo hexanoic acid (1 equiv) followed by PTSA (1 equiv) were added to 1 g of dodecanol (1 equiv) in 20 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 1.8 g of yellow color liquid was obtained. (Giving a yield of about 90%).

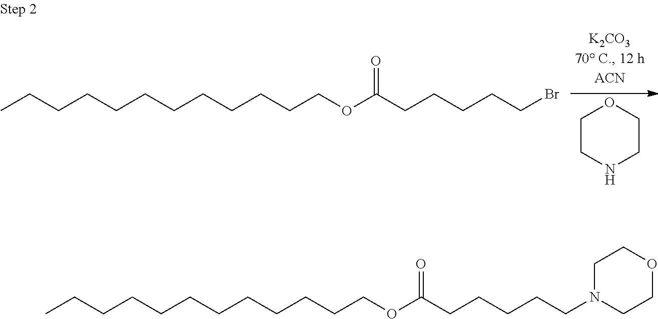

Step 2 synthesis procedure: Morpholine (1 equiv) followed by K₂CO₃ (1 equiv) were added to 1 g of bromo hexanoic ester (1 equiv) in 20 mL of ACN at room temperature and then heated at 70° C. for 12 h. The reaction mixture was filtered, then filtrate was concentrated, further crude compound was washed with hexanes to remove unreacted starting materials. 1 g of light brown liquid was obtained. (Giving a yield of about 98%).

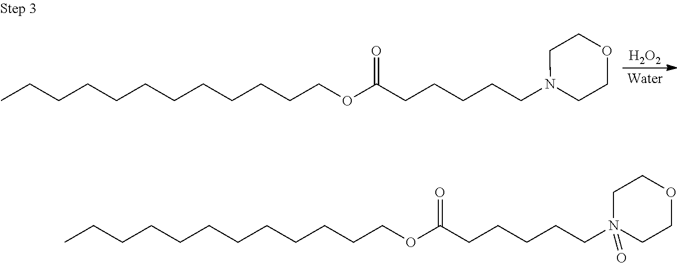

Step 3 synthesis procedure: H₂O₂ (1 equiv) was added to 0.5 g of morpholine hexanoic ester (1 equiv) in 10 mL of water at room temperature and then heated at 90° C. for 12 h. The reaction mixture was concentrated, further crude compound was washed with hexanes to remove unreacted starting materials. 0.3 g of light-yellow liquid was obtained. (Giving a yield of about 58%).

### Example 6a: Determination of Physical Properties of Surfactant 6

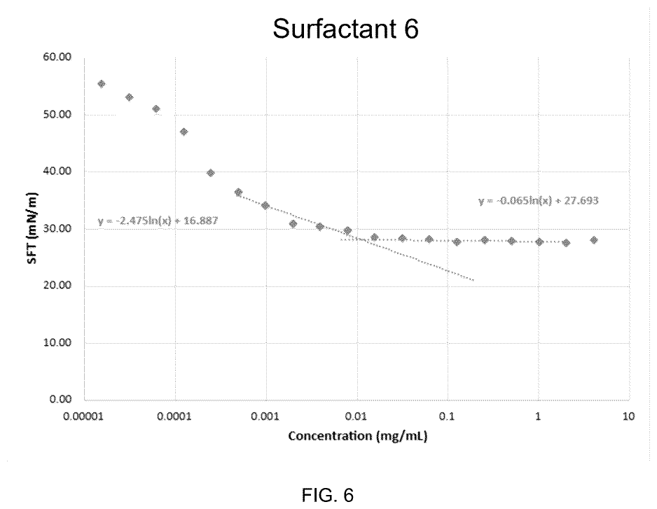

The critical micelle concentration (CMC) for Surfactant 6 was measured. From the surface tension change with concentration in water, the CMC was determined to be about 0.0293 mM (0.0113 mg/mL) at pH 4. The plateau value of minimum surface tension that can be reached by this surfactant was about 27.74 mN/m, indicating that the surfactant has outstanding interfacial activity. These results were plotted as surface tension versus concentration in FIG. 6.

The molecular parameters of surfactant 6 are summarized in Table 7:

TABLE 7

| | |
|---|---|
| CMC (mg/mL) | 0.0113 |
| CMC (mM) | 0.0293 |
| γ_{cmc} (mN/m) | 27.74 |
| 10³ × Γₘₐₓ (mmol/m²) | 5.61 |
| Åₘᵢₙ (Å²) | 29.61 |
| pH @ 8 mg/mL | 4.24 |
| pH @ 0.00098 mg/mL | 5.71 |
| prefactor? | 1 |

### Example 7: Synthesis of Surfactant 7

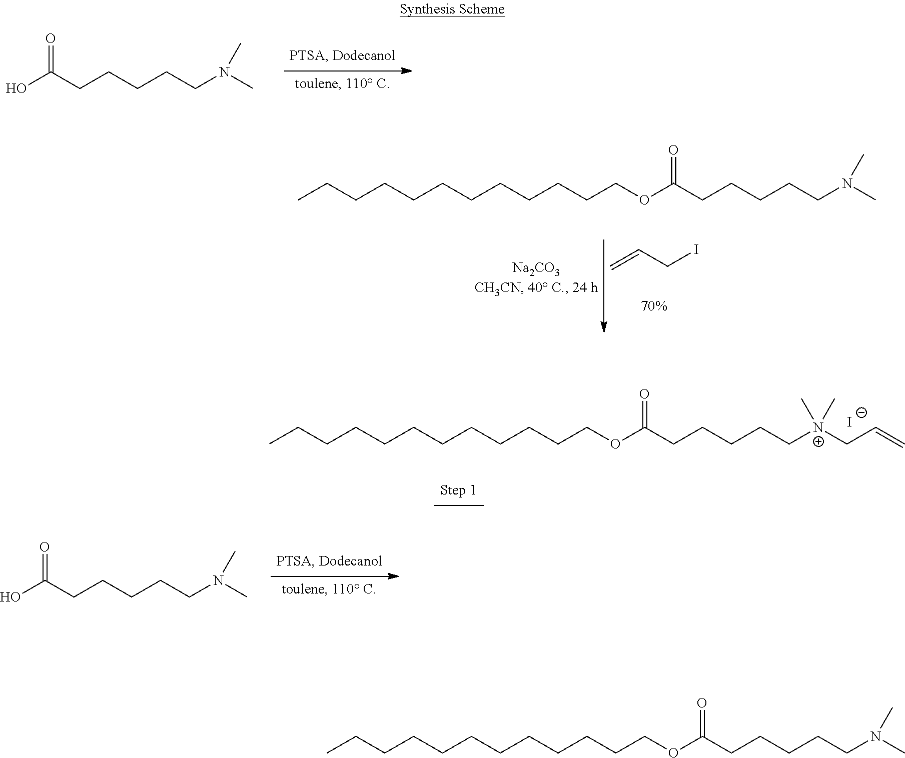

Step 1 synthesis procedure: Dimethyl amino hexanoic acid (1 equiv) followed by PTSA (1 equiv) were added to 12 g of dodecanol (1 equiv) in 100 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 22 g of brown liquid was obtained. (Giving a yield of about 91%).

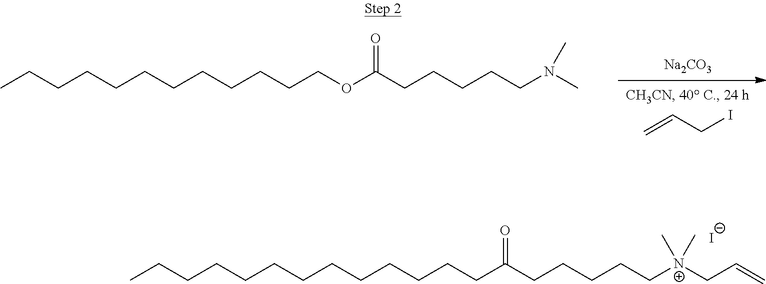

Step 2 synthesis procedure: Allyl iodide (1 equiv) followed by sodium carbonate (1 equiv) were added to 1 g of dimethyl amino hexanoic acid (1 equiv) in 20 mL of ACN at room temperature and then heated at 40° C. for 14 h. The reaction mixture was then filtered, and filtrate were concentrated, and crude compound was washed with ether (20 mL). The crude compound was dried under a high vacuum. 1.1 g of sticky brown liquid was obtained. (giving a yield of about 84%).

### Example 7a: Determination of Physical Properties of Surfactant 7

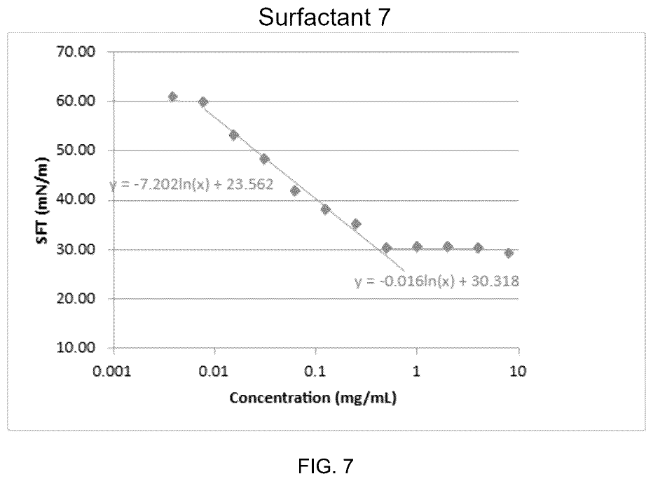

The critical micelle concentration (CMC) for Surfactant 7 was measured. From the surface tension change with concentration in water, the CMC was determined to be about 0.7886 mM (0.03906 mg/mL) at pH 7. The plateau value of minimum surface tension that can be reached by this surfactant was about 30.31 mN/m, indicating that the surfactant has outstanding interfacial activity. These results were plotted as surface tension versus concentration in FIG. 7.

The molecular parameters of surfactant 7 are summarized in Table 8:

TABLE 8

| | |
|---|---|
| CMC (mg/mL) | 0.3906 |
| CMC (mM) | 0.7886 |
| γ_{cmc} (mN/m) | 30.31 |
| 10³ × Γₘₐₓ (mmol/m²) | 1.48 |
| Åₘᵢₙ (Å²) | 112.39 |
| pH @ 0.00391 mg/mL | 7.43 |
| pH @ 8 mg/mL | 6.41 |

### Example 8: Synthesis of Surfactant 8

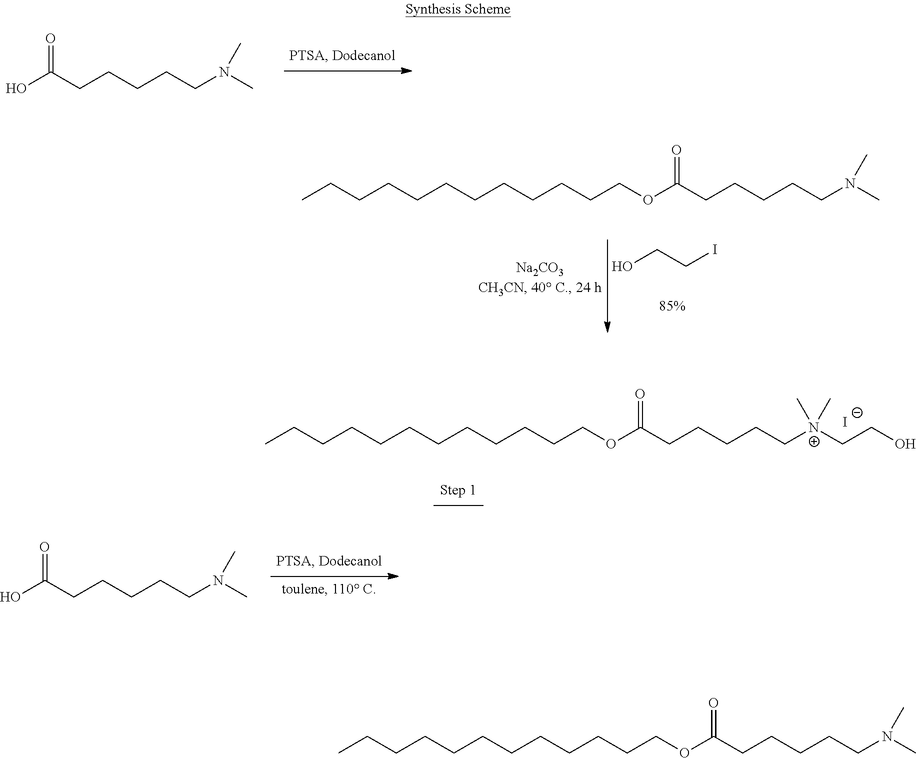

Step 1 synthesis procedure: Dimethyl amino hexanoic acid (1 equiv) followed by PTSA (1 equiv) were added to 12 g of dodecanol (1 equiv) in 100 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 22 g of brown liquid was obtained. (Giving a yield of about 91%).

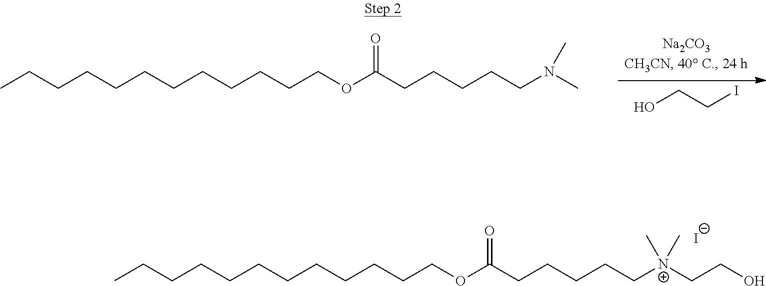

Step 2 synthesis procedure: Iodo ethanol (1 equiv) followed by sodium carbonate (1 equiv) were added to 1 g dimethyl amino hexanoic ester (1 equiv) in 20 mL of ACN at room temperature and then heated at 40° C. for 14 h. The reaction mixture was then filtered, and filtrate were concentrated, then crude compound was washed with ether (20 mL). The compound was dried under a high vacuum. 1.5 g of white solid was obtained. (giving a yield of about 80%).

### Example 8a: Determination of Physical Properties of Surfactant 8

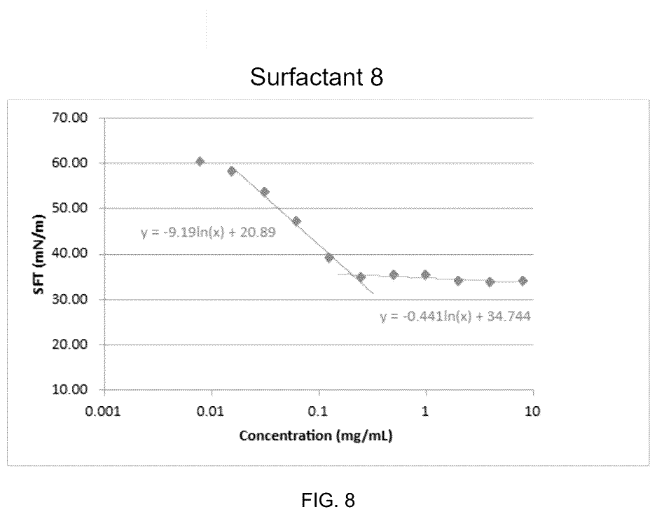

The critical micelle concentration (CMC) for Surfactant 8 was measured. From the surface tension change with concentration in water, the CMC was determined to be about 0.4109 mM (0.2053 mg/mL) at pH 6. The plateau value of minimum surface tension that can be reached by this surfactant was about 34.59 mN/m, indicating that the surfactant has outstanding interfacial activity. These results were plotted as surface tension versus concentration in FIG. 8.

The molecular parameters of surfactant 8 are summarized in Table 9:

TABLE 9

| | |
|---|---|
| CMC (mg/mL) | 0.2053 |
| CMC (mM) | 0.4109 |
| γ_{cmc} (mN/m) | 34.59 |
| 10³ × Γₘₐₓ (mmol/m²) | 1.89 |
| Åₘᵢₙ (Å²) | 88.08 |
| pH @ 0.00781 mg/mL | 6.67 |
| pH @ 8 mg/mL | 7.65 |

### Example 9: Synthesis of Surfactant 9

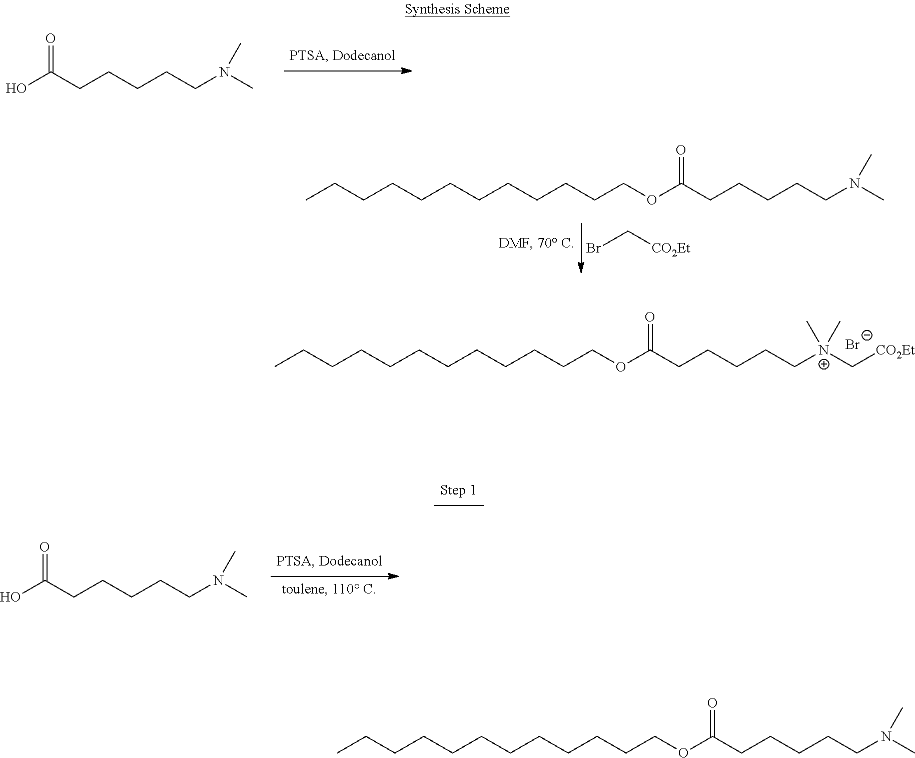

Step 1 synthesis procedure: Dimethyl amino hexanoic acid (1 equiv) followed by PTSA (1 equiv) were added to 12 g of dodecanol (1 equiv) in 100 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 22 g of brown liquid was obtained. (Giving a yield of about 91%).

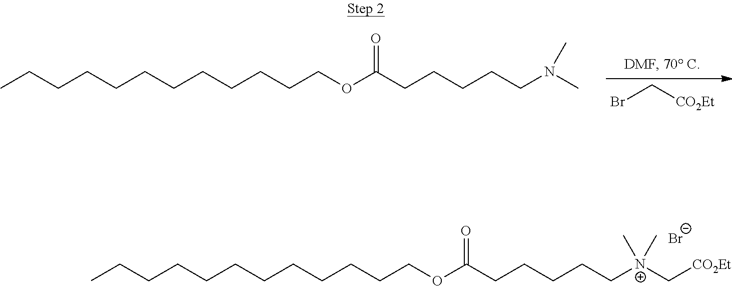

Step 2 synthesis procedure: Bromoethyl acetate (1 equiv)] was added to 1 g of dimethyl amino hexanoic ester (1 equiv) in 20 mL of DMF at room temperature and then heated at 70° C. for 14 h. The reaction mixture was then concentrated, and crude compound was washed with ether (20 mL). The compound was dried under a high vacuum. 0.9 g of sticky brown liquid was obtained. (Giving a yield of about 80%).

### Example 9a: Determination of Physical Properties of Surfactant 9

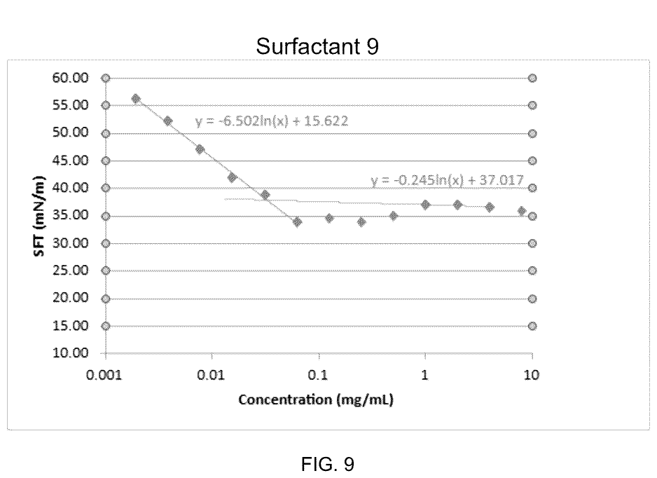

The critical micelle concentration (CMC) for Surfactant 9 was measured. From the surface tension change with concentration in water, the CMC was determined to be about 0.0662 mM (0.0327 mg/mL) at pH 7. The plateau value of minimum surface tension that can be reached by this surfactant was about 36.85 mN/m, indicating that the surfactant has outstanding interfacial activity. These results were plotted as surface tension versus concentration in FIG. 9.

The molecular parameters of surfactant 9 are summarized in Table 10:

TABLE 10

| | |
|---|---|
| CMC (mg/mL) | 0.0327 |
| CMC (mM) | 0.0662 |
| γ_{cmc} (mN/m) | 36.85 |
| 10³ × Γₘₐₓ (mmol/m²) | 1.33 |
| Åₘᵢₙ (Å²) | 124.49 |
| pH @ 0.00195 mg/mL | 7.42 |
| pH @ 8 mg/mL | 3.32 |

### Example 10: Synthesis of Surfactant 10

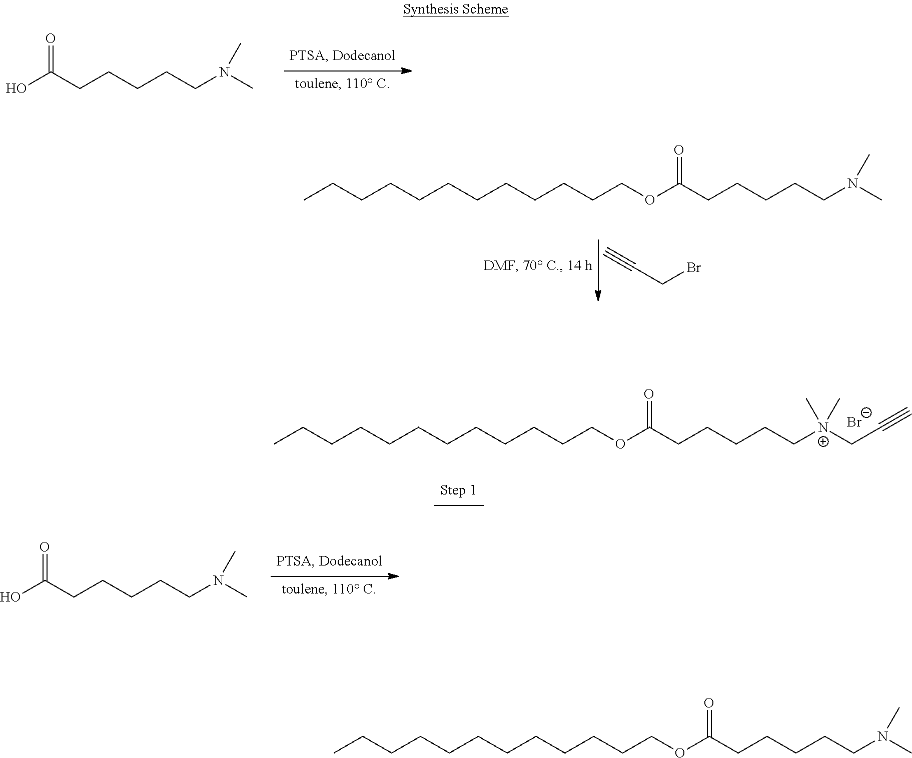

Step 1 synthesis procedure: Dimethyl amino hexanoic acid (1 equiv) followed by PTSA (1 equiv) were added to 12 g of dodecanol (1 equiv) in 100 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 22 g of brown liquid was obtained. (Giving a yield of about 91%).

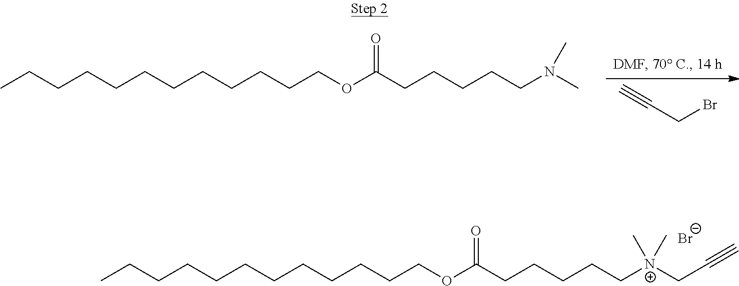

Step 2 synthesis procedure: Propargyl bromide (1 equiv) was added to 1 g of dimethyl amino hexanoic acid (1 equiv) in 20 mL of DMF at room temperature and then heated at 70° C. for 14 h. The reaction mixture was then concentrated, and crude compound was washed with ether (20 mL). The crude compound was dried under a high vacuum. 1.1 g of sticky brown liquid was obtained. (Giving a yield of about 84%).

### Example 10a: Determination of Physical Properties of Surfactant 10

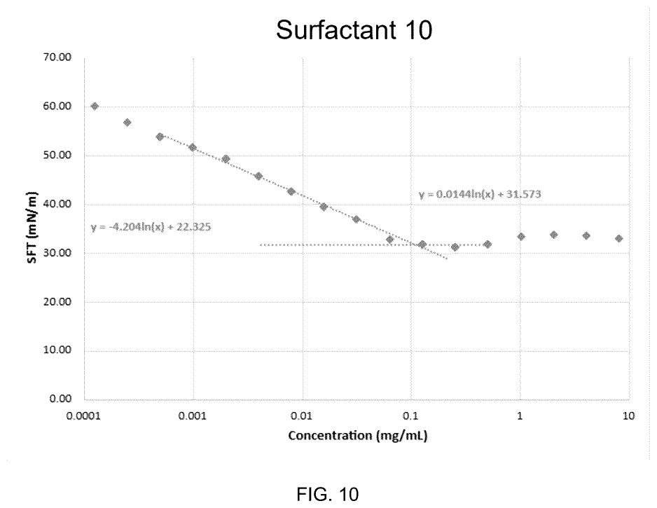

The critical micelle concentration (CMC) for Surfactant 10 was measured. From the surface tension change with concentration in water, the CMC was determined to be about 0.2463 mM (0.1100 mg/mL) at pH 4. The plateau value of minimum surface tension that can be reached by this surfactant was about 31.55 mN/m, indicating that the surfactant has outstanding interfacial activity. These results were plotted as surface tension versus concentration in FIG. 10.

The molecular parameters of surfactant 10 are summarized in Table 11:

TABLE 11

| | |
|---|---|
| CMC (mg/mL) | 0.1100 |
| CMC (mM) | 0.2463 |
| γ_{cmc} (mN/m) | 31.55 |
| 10³ × Γₘₐₓ (mmol/m²) | 0.86 |
| Åₘᵢₙ (Å²) | 192.54 |
| pH @ 8 mg/mL | 3.40 |
| pH @ 0.00012 mg/mL | 5.84 |
| prefactor? | 2 |

### Example 11: Synthesis of Surfactant 11

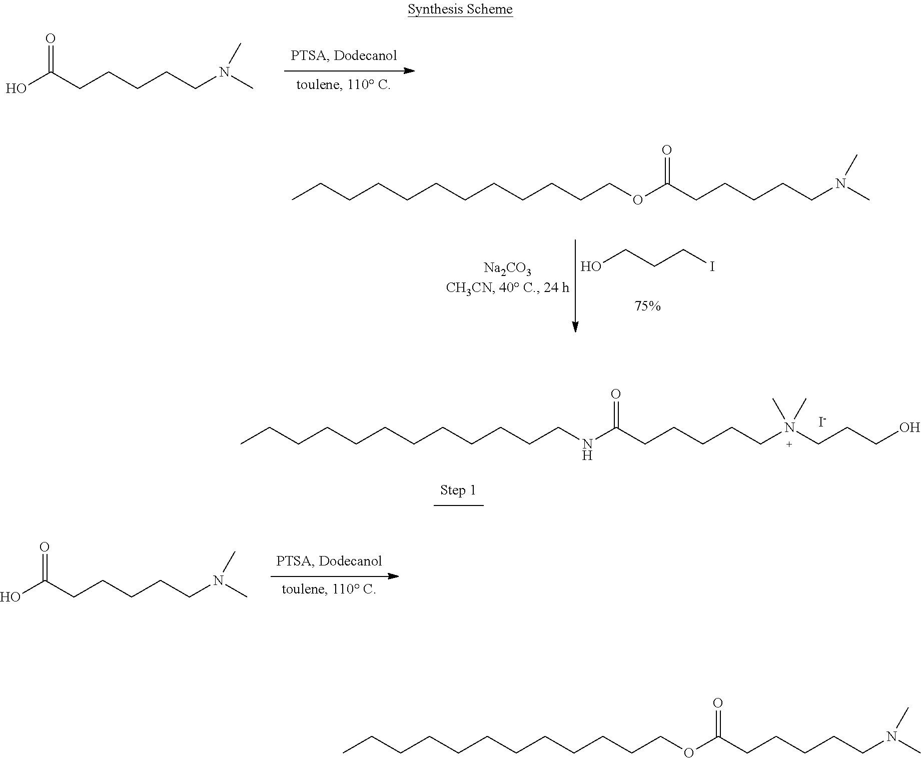

Step 1 synthesis procedure: Dimethyl amino hexanoic acid (1 equiv) followed by PTSA (1 equiv) were added to 12 g of dodecanol (1 equiv) in 100 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 22 g of brown liquid was obtained. (Giving a yield of about 91%).

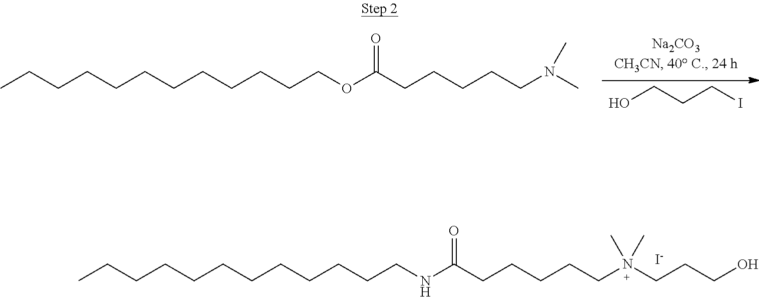

Step 2 synthesis procedure: Iodo propanol (1 equiv) followed by sodium carbonate (1 equiv) were added to 1 g of dimethyl amino hexanoic acid (1 equiv) in 20 mL of ACN at room temperature and then heated at 40° C. for 14 h. The reaction mixture was then filtered, and filtrate were concentrated, and crude compound was washed with ether (20 mL). The crude compound was dried under a high vacuum. 1.3 g of sticky brown liquid was obtained. (Giving a yield of about 81%).

### Example 11a: Determination of Physical Properties of Surfactant 11

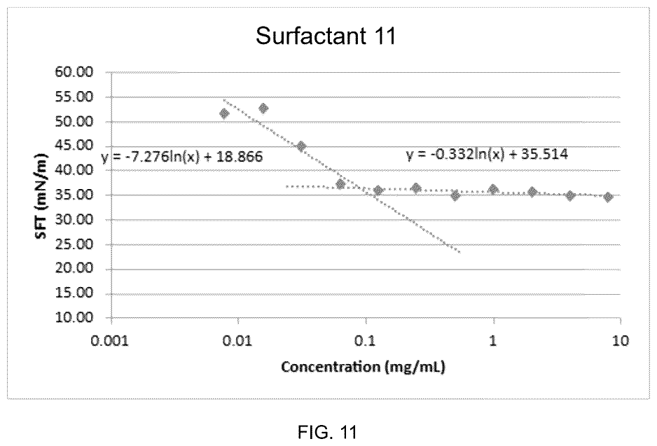

The critical micelle concentration (CMC) for Surfactant 11 was measured. From the surface tension change with concentration in water, the CMC was determined to be about 0.1771 mM (0.0909 mg/mL) at pH 6. The plateau value of minimum surface tension that can be reached by this surfactant was about 35.51 mN/m, indicating that the surfactant has outstanding interfacial activity. These results were plotted as surface tension versus concentration in FIG. 11.

The molecular parameters of surfactant 11 are summarized in Table 12:

TABLE 12

| | |
|---|---|
| CMC (mg/mL) | 0.0909 |
| CMC (mM) | 0.1771 |
| γ_{cmc} (mN/m) | 35.51 |
| 10³ × Γₘₐₓ (mmol/m²) | 1.49 |
| Åₘᵢₙ (Å²) | 111.25 |
| pH @ 0.0078 mg/mL | 5.76 |
| pH @ 8 mg/mL | 5.36 |
| prefactor | 2 |

### Example 12: Synthesis of Surfactant 12

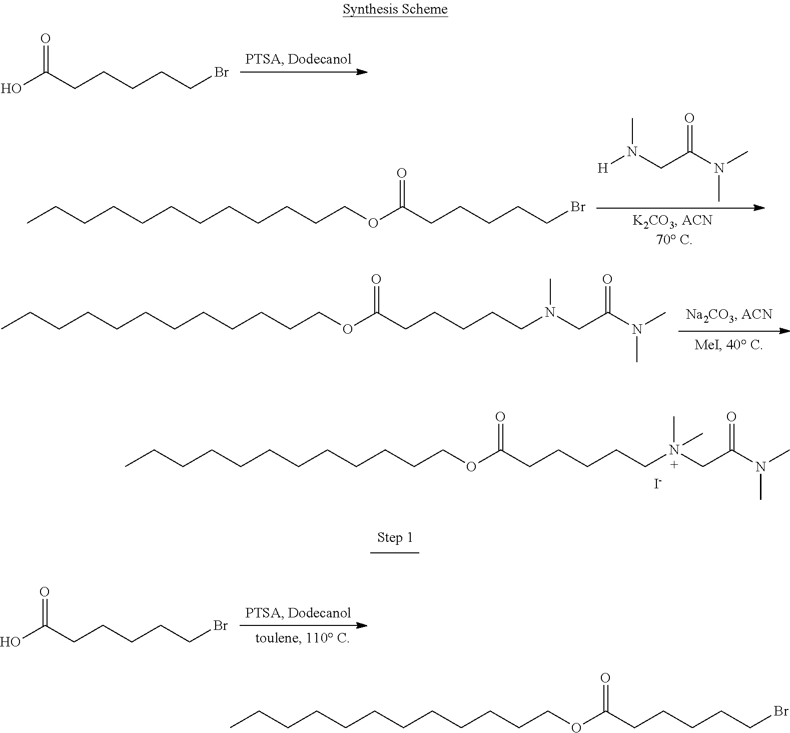

Step 1 synthesis procedure: Bromo hexanoic acid (1 equiv) followed by PTSA (1 equiv) were added to 1 g of dodecanol (1 equiv) in 20 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 1.8 g of yellow color liquid was obtained. (Giving a yield of about 90%).

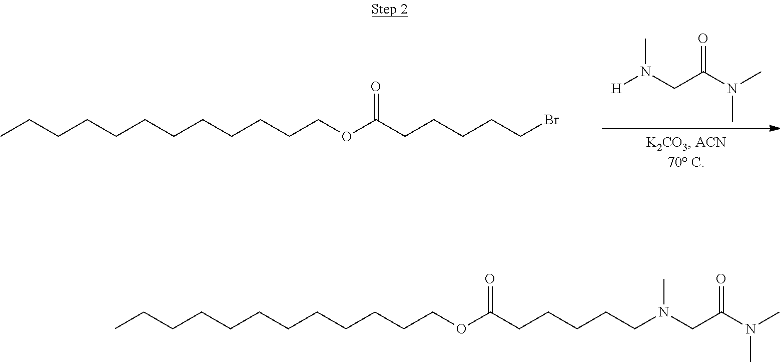

Step 2 synthesis procedure: Methyl amine starting material (1 equiv) followed by potassium carbonate (1 equiv) were added to 1 g bromo hexanoic ester (1 equiv) in 20 mL of ACN at room temperature and then heated at 70° C. for 14 h. The reaction mixture was then filtered, and filtrate were concentrated, then crude compound was washed with ether (20 mL). The compound was dried under a high vacuum. 0.9 g of sticky liquid was obtained. (Giving a yield of about 82%).

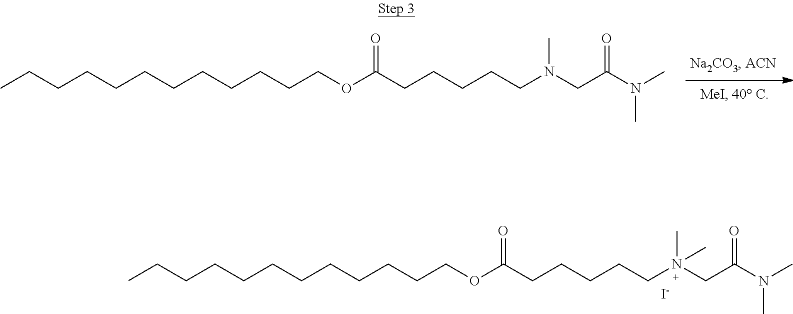

Step 3 synthesis procedure: Iodo methane (1 equiv) followed by sodium carbonate (1 equiv) were added to 1 g of the above hexanoic ester (1 equiv) in 20 mL of ACN at room temperature and then heated at 40° C. for 14 h. The reaction mixture was then filtered, and filtrate were concentrated, then crude compound was washed with ether (20 mL). The compound was dried under a high vacuum. 0.9 g of sticky liquid was obtained. (Giving a yield of about 82%).

### Example 12a: Determination of Physical Properties of Surfactant 12

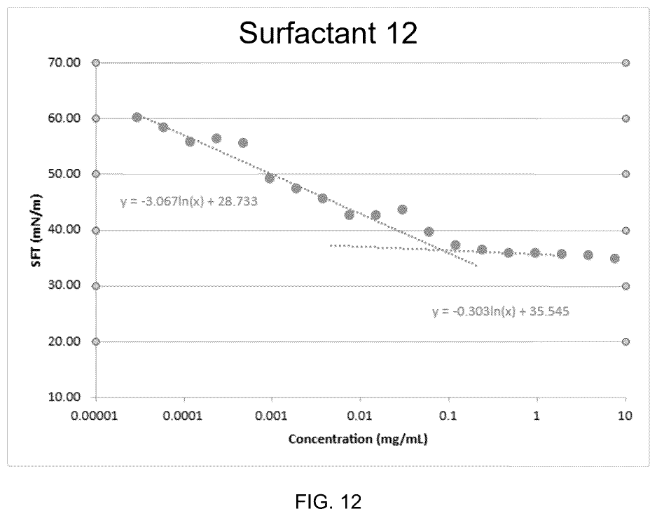

The critical micelle concentration (CMC) for Surfactant 12 was measured. From the surface tension change with concentration in water, the CMC was determined to be about 0.1573 mM (0.0850 mg/mL) at pH 6. The plateau value of minimum surface tension that can be reached by this surfactant was about 35.65 mN/m, indicating that the surfactant has outstanding interfacial activity. These results were plotted as surface tension versus concentration in FIG. 12.

The molecular parameters of surfactant 12 are summarized in Table 13:

TABLE 13

| | |
|---|---|
| CMC (mg/mL) | 0.0850 |
| CMC (mM) | 0.1573 |
| γ_{cmc} (mN/m) | 35.65 |
| 10³ × Γₘₐₓ (mmol/m²) | 0.63 |
| Åₘᵢₙ (Å²) | 263.92 |
| pH @ 0.002 mg/mL | 5.83 |
| pH @ 8 mg/mL | 8.45 |
| prefactor | 2 |

### Example 13: Synthesis of Surfactant 13

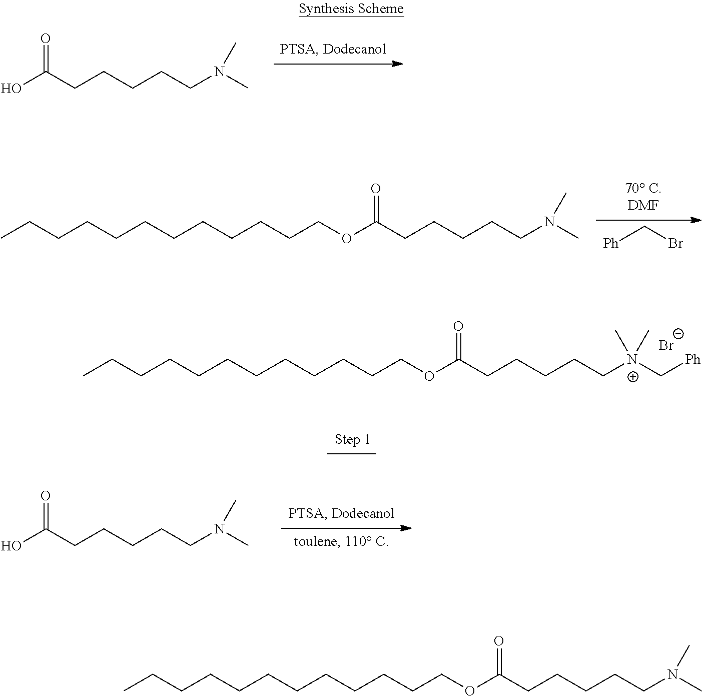

Step 1 synthesis procedure: Dimethyl Amino hexanoic acid (1 equiv) followed by PTSA (1 equiv) were added to 12 g of dodecanol (1 equiv) in 100 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 22 g of brown liquid was obtained. (Giving a yield of about 91%).

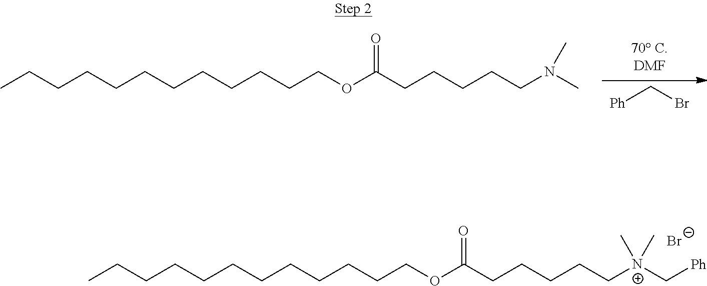

Synthesis procedure: Benzyl bromide (1 equiv) was added to 2.5 g of dimethyl amino hexanoic ester (1 equiv) in 20 mL of DMF at room temperature and then heated at 70° C. for 14 h. The reaction mixture was then concentrated, and crude compound was washed with ether (20 mL). The compound was dried under a high vacuum. 3 g of white solid was obtained. (Giving a yield of about 80%).

### Example 13a: Determination of Physical Properties of Surfactant 13

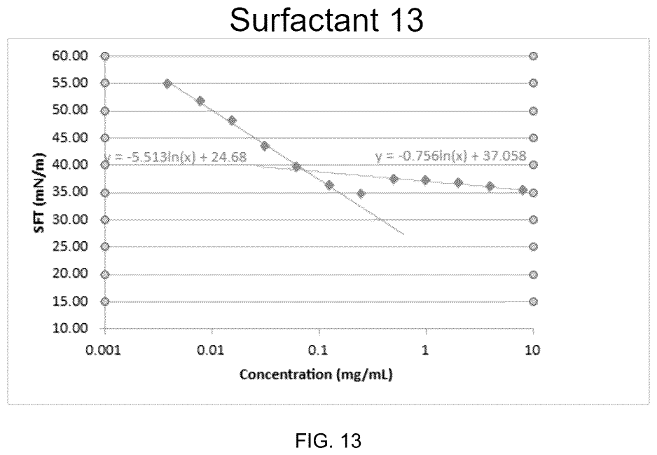

The critical micelle concentration (CMC) for Surfactant 13 was measured. From the surface tension change with concentration in water, the CMC was determined to be about 0.1487 mM (0.0741 mg/mL) at pH 6. The plateau value of minimum surface tension that can be reached by this surfactant was about 36.53 mN/m, indicating that the surfactant has outstanding interfacial activity. These results were plotted as surface tension versus concentration in FIG. 13.

The molecular parameters of surfactant 13 are summarized in Table 14:

TABLE 14

| | |
|---|---|
| CMC (mg/mL) | 0.0741 |
| CMC (mM) | 0.1487 |
| γ_{cmc} (mN/m) | 36.53 |
| 10³ × Γₘₐₓ (mmol/m²) | 1.13 |
| Åₘᵢₙ (Å²) | 146.83 |
| pH @ 0.00391 mg/mL | 5.75 |
| pH @ 8 mg/mL | 6.00 |
| prefactor? | |

### Example 14: Synthesis of Surfactant 14

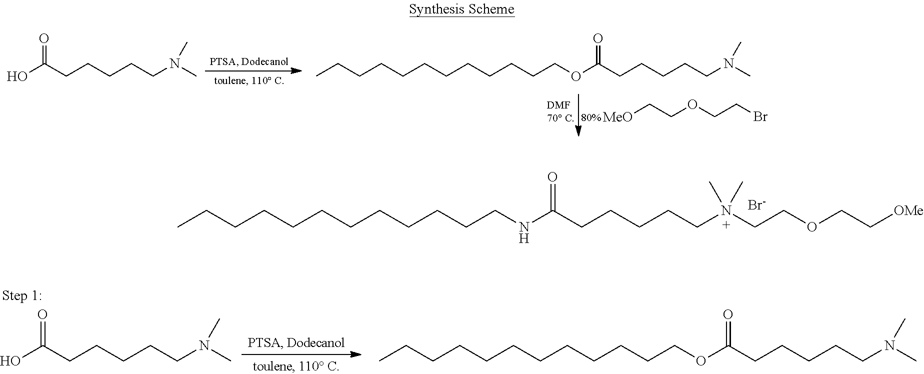

Step 1 synthesis procedure: Dimethyl amino hexanoic acid (1 equiv) followed by PTSA (1 equiv) were added to 12 g of dodecanol (1 equiv) in 100 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 22 g of brown liquid was obtained. (Giving a yield of about 91%).

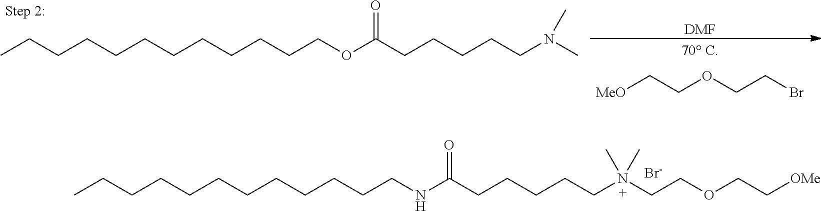

Step 2 synthesis procedure: Methoxy ethoxy bromide (1 equiv) was added to 1 g of dimethyl amino hexanoic acid (1 equiv) in 20 mL of DMF at room temperature and then heated at 70° C. for 14 h. The reaction mixture was then concentrated, and crude compound was washed with ether (20 mL). The crude compound was dried under a high vacuum give 1.2 g of sticky brown liquid was obtained. (Giving a yield of about 77%).

### Example 14a: Determination of Physical Properties of Surfactant 14

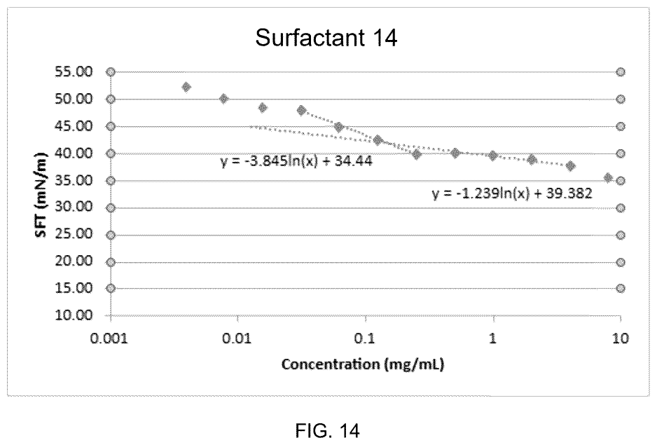

The critical micelle concentration (CMC) for Surfactant 14 was measured. From the surface tension change with concentration in water, the CMC was determined to be about 0.2940 mM (0.1501 mg/mL) at pH 6. The plateau value of minimum surface tension that can be reached by this surfactant was about 38.95 mN/m, indicating that the surfactant has outstanding interfacial activity. These results were plotted as surface tension versus concentration in FIG. 14.

The molecular parameters of surfactant 14 are summarized in Table 15:

TABLE 15

| | |
|---|---|
| CMC (mg/mL) | 0.1501 |
| CMC (mM) | 0.2940 |
| γ_{cmc} (mN/m) | 38.95 |
| 10³ × Γₘₐₓ (mmol/m²) | 0.79 |
| Åₘᵢₙ (Å²) | 210.52 |
| pH @ 0.0039 mg/mL | 5.77 |
| pH @ 8 mg/mL | 4.83 |
| prefactor? | 2 |

### Example 15: Synthesis of Surfactant 15

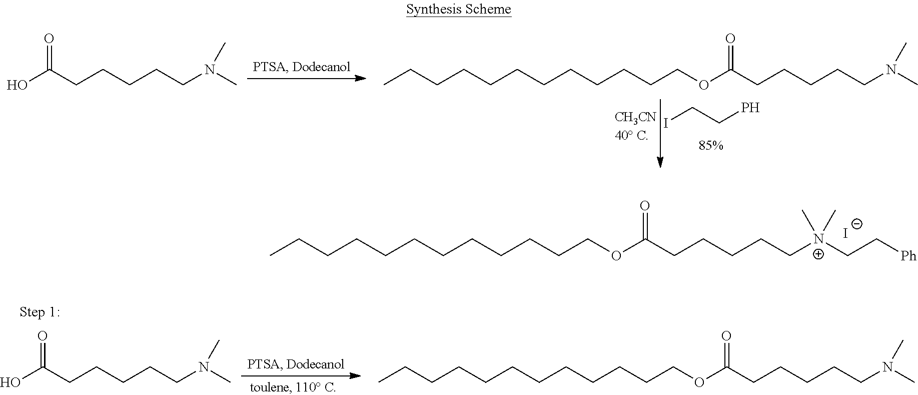

Step 1 synthesis procedure: Dimethyl amino hexanoic acid (1 equiv) followed by PTSA (1 equiv) were added to 12 g of dodecanol (1 equiv) in 100 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 22 g of brown liquid was obtained. (Giving a yield of about 91%).

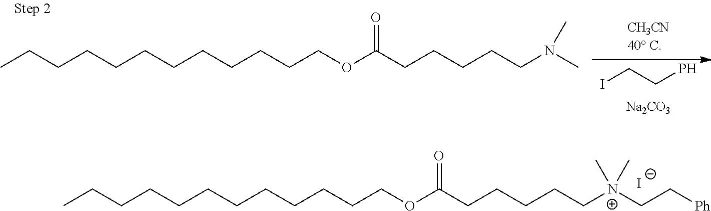

Step 2 synthesis procedure: Phenyl ethyl iodide (1 equiv) followed by sodium carbonate (1 equiv) were added to 1 g of dimethyl amino hexanoic ester (1 equiv) in 20 mL of ACN at room temperature and then heated at 40° C. for 14 h. The reaction mixture was then filtered, and filtrate was concentrated, crude compound was washed with ether (20 mL). The compound was dried under a high vacuum. 1.1 g of yellow solid was obtained. (giving a yield of about 85%).

### Example 15a: Determination of Physical Properties of Surfactant 15

Surfactant 15 has low solubility in water at room temperature, which means that basic surfactant properties of the compounds cannot be measured under the typical conditions that have been used to measure the other surfactant compounds described hereunder. Under heating, the molecule became slightly soluble in water, but it remained a challenge to attain the typical 8 mg/mL concentration required for the usual measurements to be taken.

It is however, believed that the molecule may be more soluble in lipids, rendering it potentially a useful for making a “reverse” type of oil-in-water emulsion, or can be used to prepare foams that based on lipid-air interface behaviors.

### Example 16: Synthesis of Surfactant 16

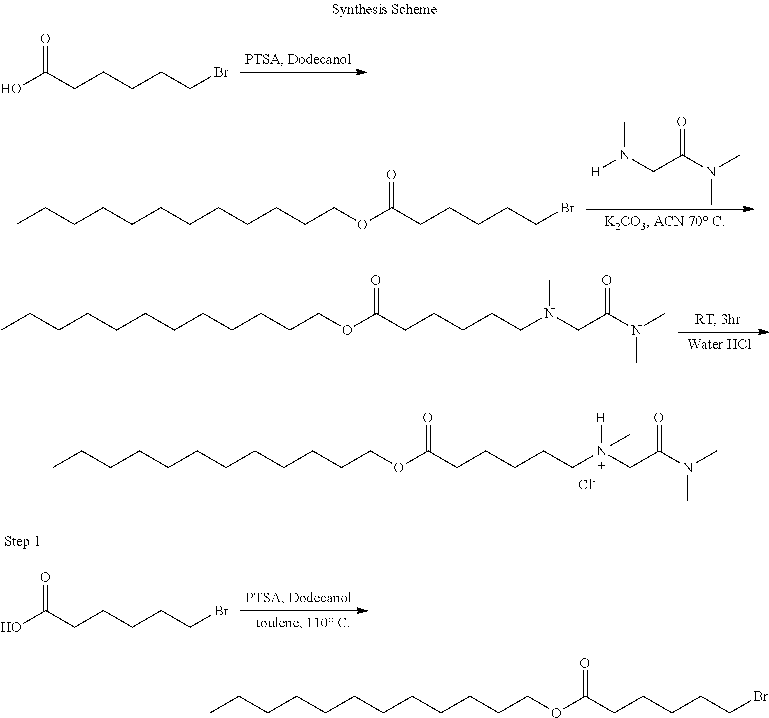

Step 1 synthesis procedure: Bromo hexanoic acid (1 equiv) followed by PTSA (1 equiv) were added to 1 g of dodecanol (1 equiv) in 20 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 1.8 g of yellow color liquid was obtained. (Giving a yield of about 90%).

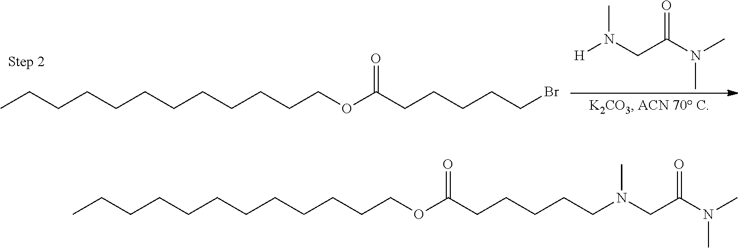

Step 2 synthesis procedure: Methyl amine starting material (1 equiv) followed by potassium carbonate (1 equiv) were added to 1 g bromo hexanoic ester (1 equiv) in 20 mL of ACN at room temperature and then heated at 70° C. for 14 h. The reaction mixture was then filtered, and filtrate were concentrated, then crude compound was washed with ether (20 mL). The compound was dried under a high vacuum. 0.9 g of sticky liquid was obtained. (Giving a yield of about 82%).

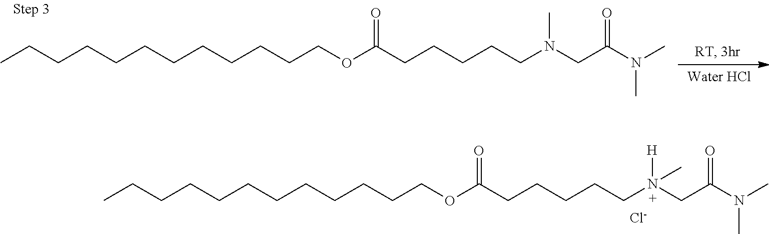

Step 3 synthesis procedure: Con. HCl (1 equiv) was added to 1 g of the above hexanoic ester (1 equiv) in 10 mL of water at room temperature and then stirred for 14 h. The reaction mixture was then concentrated, crude compound was washed with ether (20 mL). The compound was dried under a high vacuum. 0.92 g of sticky liquid was obtained. (Giving a yield of about 83%).

### Example 16a: Determination of Physical Properties of Surfactant 16

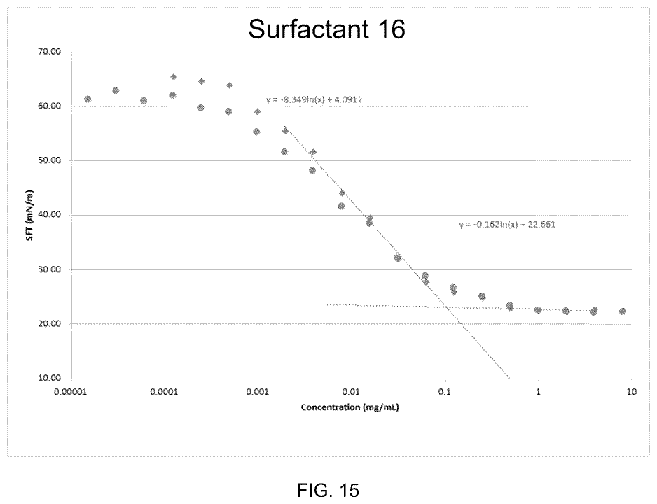

The critical micelle concentration (CMC) for Surfactant 16 was measured. From the surface tension change with concentration in water, the CMC was determined to be about 0.2379 mM (0.1035 mg/mL) at pH 6. The plateau value of minimum surface tension that can be reached by this surfactant was about 22.6 mN/m, indicating that the surfactant has outstanding interfacial activity. These results were plotted as surface tension versus concentration in FIG. 15.

The molecular parameters of surfactant 16 are summarized in Table 16:

TABLE 16

| | (Mar. 29, 2022) | | (May 27, 2022) |
|---|---|---|---|
| CMC (mg/mL) | 0.1035 | CMC (mg/mL) | 0.1035 |
| CMC (mM) | 0.2379 | CMC (mM) | 0.2379 |
| γ_{cmc} (mN/m) | 22.61 | γ_{cmc} (mN/m) | 22.8 |
| 10³ × | 1.71 | 10³ × | 1.71 |
| Γₘₐₓ (mmol/m²) | | Γₘₐₓ (mmol/m²) | |
| Åₘᵢₙ (Å²) | 96.95 | Åₘᵢₙ (Å²) | 96.95 |
| pH @ 0.0001 | 5.57 | pH @ 0.000015 | 5.79 |
| mg/mL | | mg/mL | |
| pH @ 8 mg/mL | 2.28 | pH @ 8 mg/mL | 2.11 |
| prefactor | 2 | prefactor | 2 |

### Example 17: Synthesis of Surfactant 17

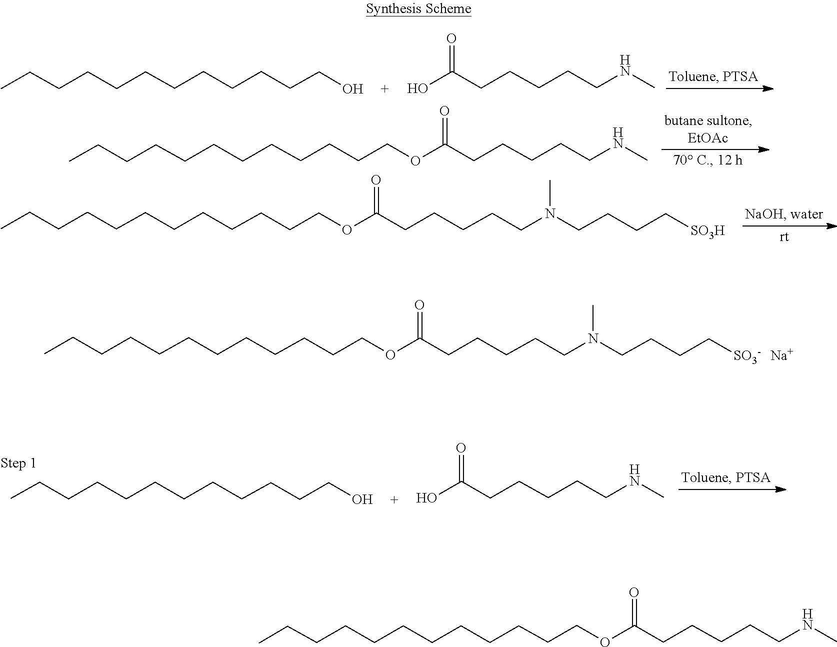

Step 1 synthesis procedure: 6-amino methyl hexanoic acid (1 equiv) followed by PTSA (1 equiv) were added to 12.8 g of dodecanol (1 equiv) in 20 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 16 g of brown liquid was obtained. (Giving a yield of about 76%).

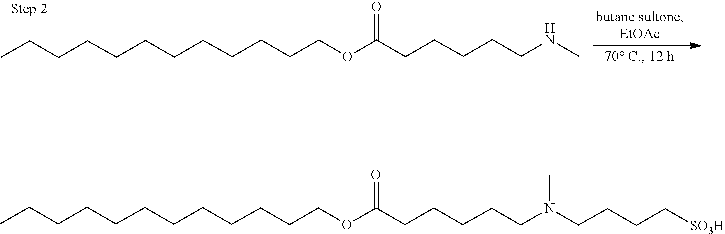

Step 2 synthesis procedure: Butane sultone (1 equiv) was added to 1.6 g of amino hexanoic ester (1 equiv) in 20 mL of EtOAc at room temperature and then refluxed for 14 h. The reaction mixture was then concentrated, and crude compound was washed with ether (20 mL). The crude compound was dried under a high vacuum and used compound for next step without any further purification. 1.2 g of sticky brown liquid was obtained. (giving a yield of about 54%).

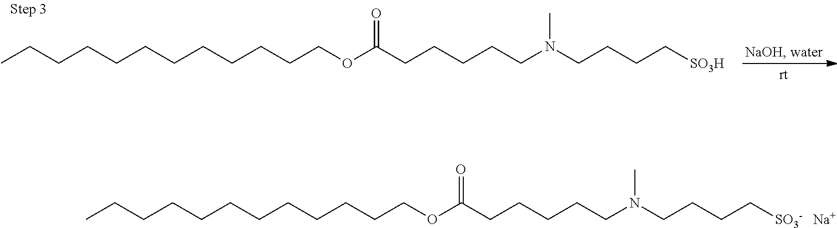

Step 3 synthesis procedure: NaOH (1 equiv) was added to 1.1 g of sulfonate (1 equiv) in 10 mL of water at room temperature and stirred for 3 h. The reaction mixture was then concentrated, and crude compound was washed with ether and dried under high vacuum. 1.0 g of yellow solid was obtained. (Giving a yield of about 90%).

### Example 17a: Determination of Physical Properties of Surfactant 17

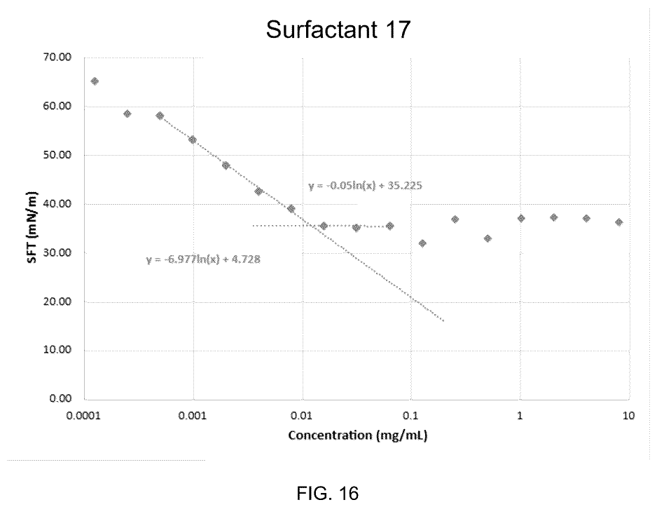

The critical micelle concentration (CMC) for Surfactant 17 was measured. From the surface tension change with concentration in water, the CMC was determined to be about 0.0260 mM (0.0122 mg/mL) at pH 11. The plateau value of minimum surface tension that can be reached by this surfactant was about 35.40 mN/m, indicating that the surfactant has outstanding interfacial activity. These results were plotted as surface tension versus concentration in FIG. 16.

The molecular parameters of surfactant 17 are summarized in Table 17:

TABLE 17

| | |
|---|---|
| CMC (mg/mL) | 0.0122 |
| CMC (mM) | 0.0260 |
| γ_{cmc} (mN/m) | 35.40 |
| 10³ × Γₘₐₓ (mmol/m²) | 1.43 |
| Åₘᵢₙ (Å²) | 116.02 |
| pH @ 8 mg/mL | 11.76 |
| pH @ 0.00012 mg/mL | 5.99 |
| prefactor? | 2 |

### Example 18: Synthesis of Surfactant 18

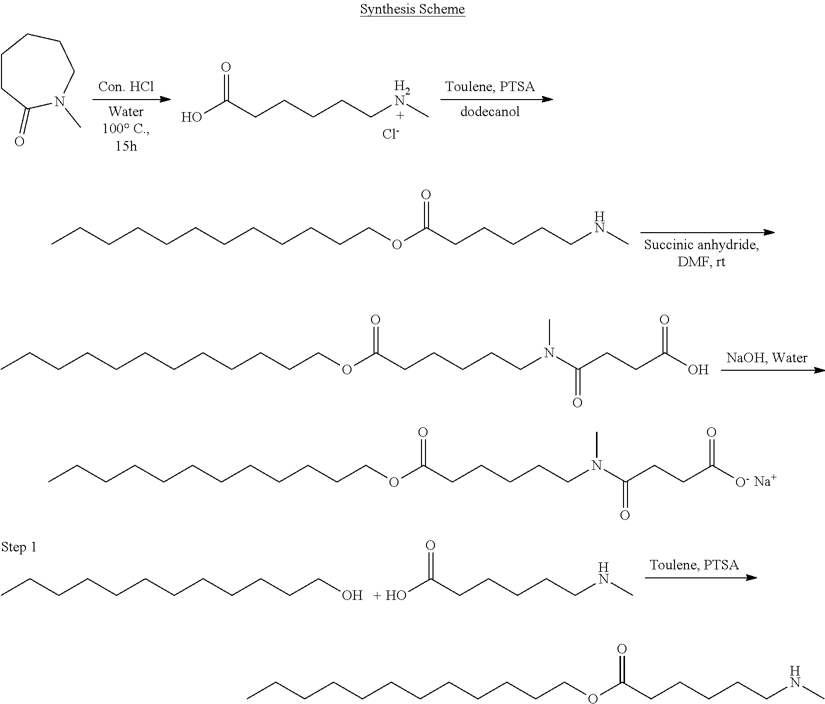

Step 1 synthesis procedure: 6-amino methyl hexanoic acid (1 equiv) followed by PTSA (1 equiv) were added to 12.8 g of dodecanol (1 equiv) in 20 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 16 g of brown liquid obtained was obtained. (Giving a yield of about 76%).

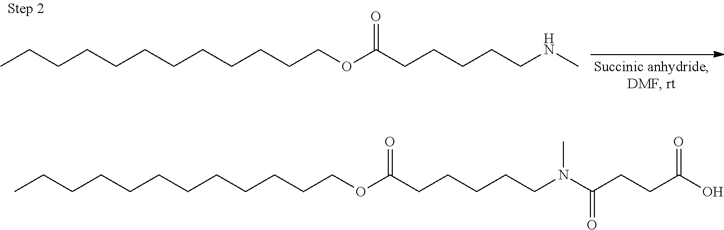

Step 2 synthesis procedure: Succinic anhydride (1 equiv) was added to 1 g of amino hexanoic ester (1 equiv) in 20 mL of DMF at room temperature and then stirred for 14 h. The reaction mixture was then concentrated, and crude compound was poured into ice water. The white solid precipitate was filtered, dried under high vacuum and used solid compound for next step without any further purification. 1.2 g of white solid was obtained. (giving a yield of about 92%).

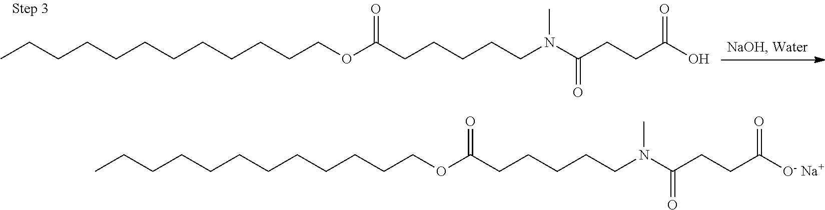 [SMILES: CCCCCCCCCCCCOC(=O)CCCCCN(C)C(=O)CCC(=O)O ; CCCCCCCCCCCCOC(=O)CCCCCN(C)C(=O)CCC(=O)[O-] ; [Na+]]

Step 3 synthesis procedure: NaOH (1 equiv) was added to 1.2 g of acid compound (1 equiv) in 10 mL of water at room temperature and stirred for 3 h. The reaction mixture was then concentrated, and crude compound was washed with ether and dried under high vacuum. 1.21 g of white solid was obtained. (Giving a yield of about 97%).

### Example 18a: Determination of Physical Properties of Surfactant 18

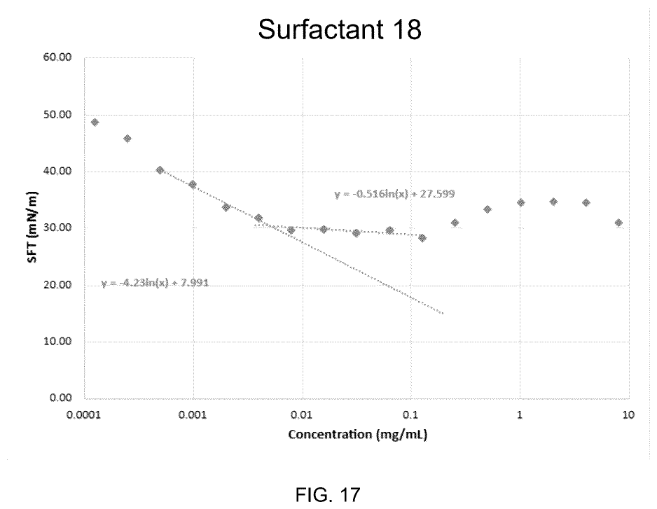

The critical micelle concentration (CMC) for Surfactant 18 was measured. From the surface tension change with concentration in water, the CMC was determined to be about 0.0117 mM (0.0051 mg/mL) at pH 8. The plateau value of minimum surface tension that can be reached by this surfactant was about 29.21 mN/m, indicating that the surfactant has outstanding interfacial activity. These results were plotted as surface tension versus concentration in FIG. 17.

The molecular parameters of surfactant 18 are summarized in Table 18:

TABLE 18

| | |
|---|---|
| CMC (mg/mL) | 0.0051 |
| CMC (mM) | 0.0117 |
| γ_{cmc} (mN/m) | 29.21 |
| 10³ × Γₘₐₓ (mmol/m²) | 0.87 |
| Åₘᵢₙ (Å²) | 191.36 |
| pH @ 8 mg/mL | 8.02 |
| pH @ 0.00012 mg/mL | 5.87 |
| prefactor? | 2 |

### Example 19: Synthesis of Surfactant 19

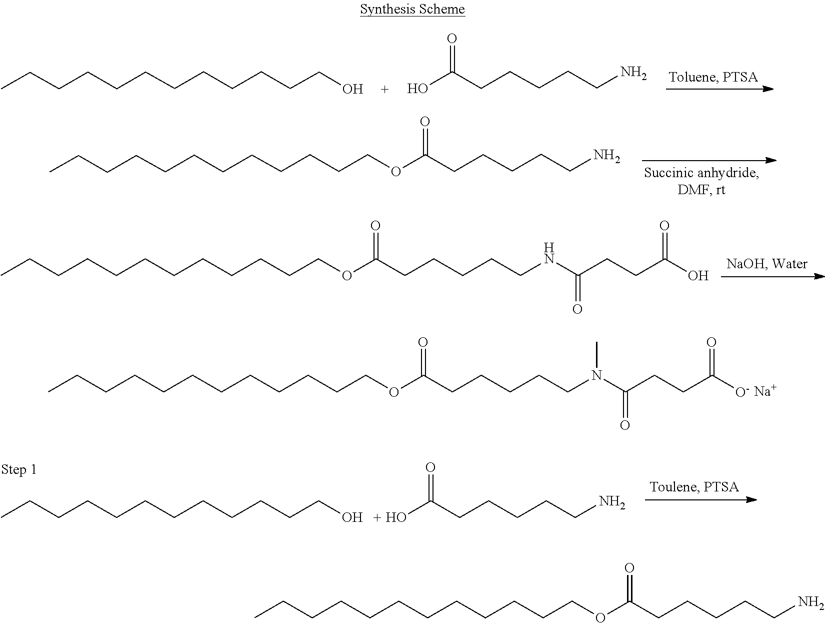

Step 1 synthesis procedure: 6-amino hexanoic acid (1 equiv) followed by PTSA (1 equiv) were added to 4 g of dodecanol (1 equiv) in 20 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 6.4 g of brown liquid was obtained. (Giving a yield of about 40%).

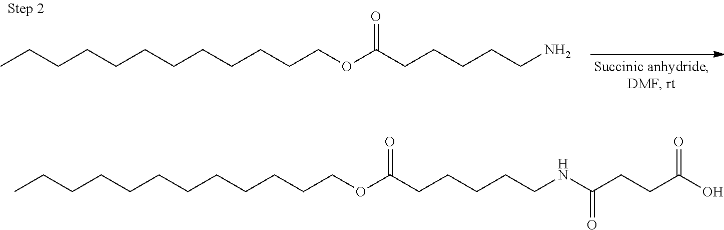 [SMILES: CCCCCCCCCCCCOC(=O)CCCCCN ; CCCCCCCCCCCCOC(=O)CCCCCNC(=O)CCC(=O)O]

Step 2 synthesis procedure: Succinic anhydride (1 equiv) was added to 1 g of amino hexanoic ester (1 equiv) in 20 mL of DMF at room temperature and then stirred for 14 h. The reaction mixture was then concentrated, and crude compound was poured into ice water. The white solid precipitate was filtered, dried under high vacuum and used solid compound for next step without any further purification. 1.3 g of white solid was obtained. (Giving a yield of about 95%).

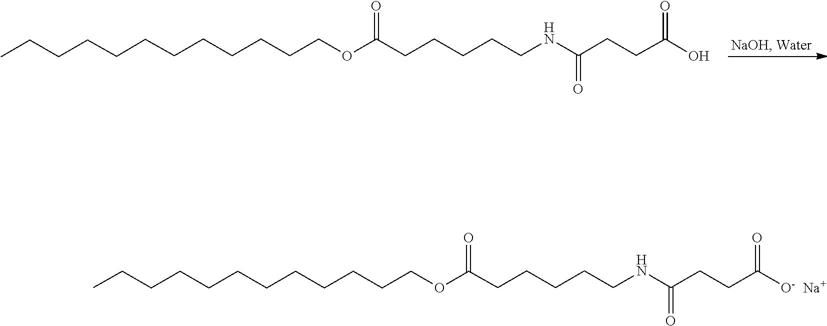 [SMILES: CCCCCCCCCCCCOC(=O)CCCCCNC(=O)CCC(=O)O ; CCCCCCCCCCCCOC(=O)CCCCCNC(=O)CCC(=O)[O-] ; [Na+]]

Step 3 synthesis procedure: NaOH (1 equiv) was added to 1.2 g of acid compound (1 equiv) in 10 mL of water at room temperature and stirred for 3 h. The reaction mixture was then concentrated, and crude compound was washed with ether and dried under high vacuum. 1.18 g of white solid was obtained. (Giving a yield of about 90%).

### Example 19a: Determination of Physical Properties of Surfactant 19

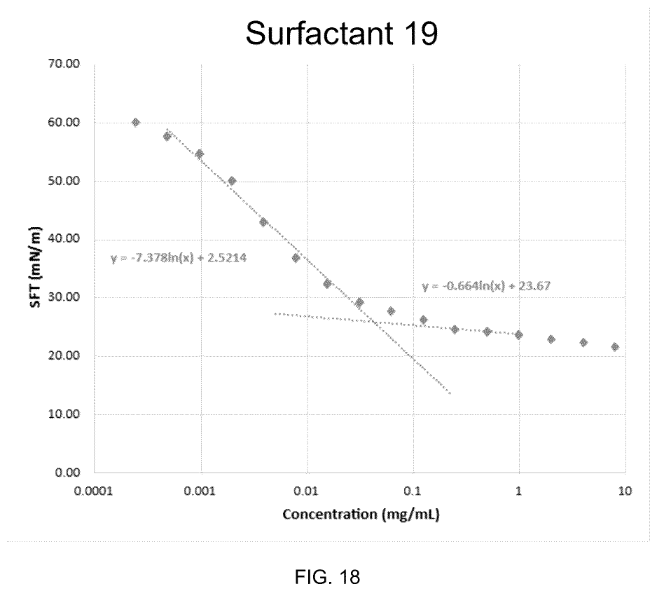

The critical micelle concentration (CMC) for Surfactant 19 was measured. From the surface tension change with concentration in water, the CMC was determined to be about 0.0969 mM (0.0409 mg/mL) at pH 9. The plateau value of minimum surface tension that can be reached by this surfactant was about 23.9 mN/m, indicating that the surfactant has outstanding interfacial activity. These results were plotted as surface tension versus concentration in FIG. 18.

The molecular parameters of surfactant 19 are summarized in Table 19:

TABLE 19

| | |
|---|---|
| CMC (mg/mL) | 0.0409 |
| CMC (mM) | 0.0969 |
| γ_{cmc} (mN/m) | 23.90 |
| 10³ × Γₘₐₓ (mmol/m²) | 1.51 |
| Åₘᵢₙ (Å²) | 109.71 |
| pH @ 8 mg/mL | 9.89 |
| pH @ 0.00002 mg/mL | 9.31 |
| prefactor? | 2 |

### Example 20: Synthesis of Surfactant 20

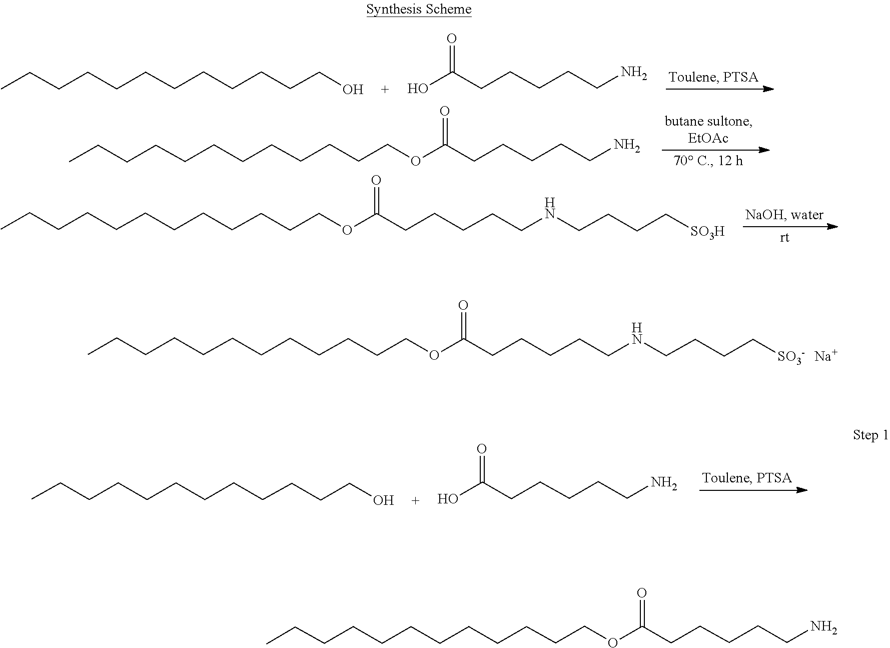

Step 1 synthesis procedure: 6-amino hexanoic acid (1 equiv) followed by PTSA (1 equiv) were added to 4 g of dodecanol (1 equiv) in 20 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 6.4 g of brown liquid was obtained. (Giving a yield of about 40%).

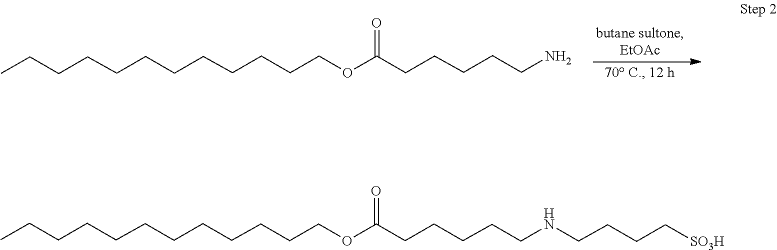 [SMILES: CCCCCCCCCCCCOC(=O)CCCCCN ; CCCCCCCCCCCCOC(=O)CCCCCNCCCCS(=O)(=O)O]

Step 2 synthesis procedure: Butane sultone (1 equiv) was added to 0.8 g of amino hexanoic ester (1 equiv) in 20 mL of EtOAc at room temperature and then refluxed for 14 h. The reaction mixture was then concentrated, and crude compound was washed with ether (20 mL). The crude compound was dried under a high vacuum and used compound for next step without any further purification. 0.7 g of sticky brown liquid was obtained. (Giving a yield of about 55%).

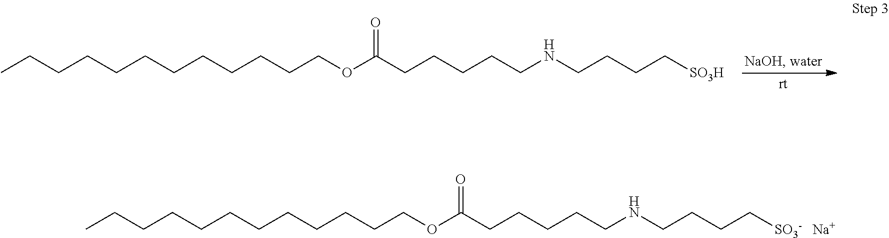

Step 3 synthesis procedure: NaOH (1 equiv) was added to 1.1 g of sulfonate (1 equiv) in 10 mL of water at room temperature and stirred for 3 h. The reaction mixture was then concentrated, and crude compound was washed with ether and dried under high vacuum. 1.05 g of yellow solid was obtained. (Giving a yield of about 92%).

### Example 20a: Determination of Physical Properties of Surfactant 20

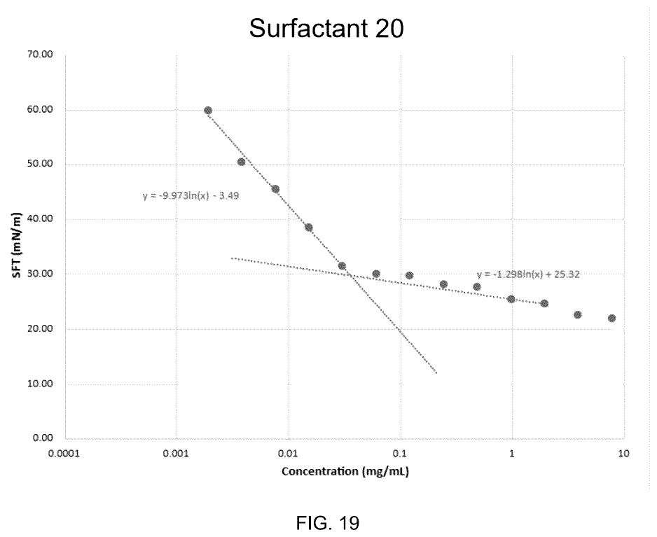

The critical micelle concentration (CMC) for Surfactant 20 was measured. From the surface tension change with concentration in water, the CMC was determined to be about 0.0789 mM (0.0361 mg/mL) at pH 12. The plateau value of minimum surface tension that can be reached by this surfactant was about 24.87 mN/m, indicating that the surfactant has outstanding interfacial activity. These results were plotted as surface tension versus concentration in FIG. 19.

The molecular parameters of surfactant 20 are summarized in Table 20:

TABLE 20

| | |
|---|---|
| CMC (mg/mL) | 0.0361 |
| CMC (mM) | 0.0789 |
| γ_{cmc} (mN/m) | 24.87 |
| 10³ × Γₘₐₓ (mmol/m²) | 2.05 |
| Åₘᵢₙ (Å²) | 81.15 |
| pH @ 8 mg/mL | 12.30 |
| pH @ 0.00098 mg/mL | 6.61 |
| prefactor? | 2 |

### Example 21: Synthesis of Surfactant 21

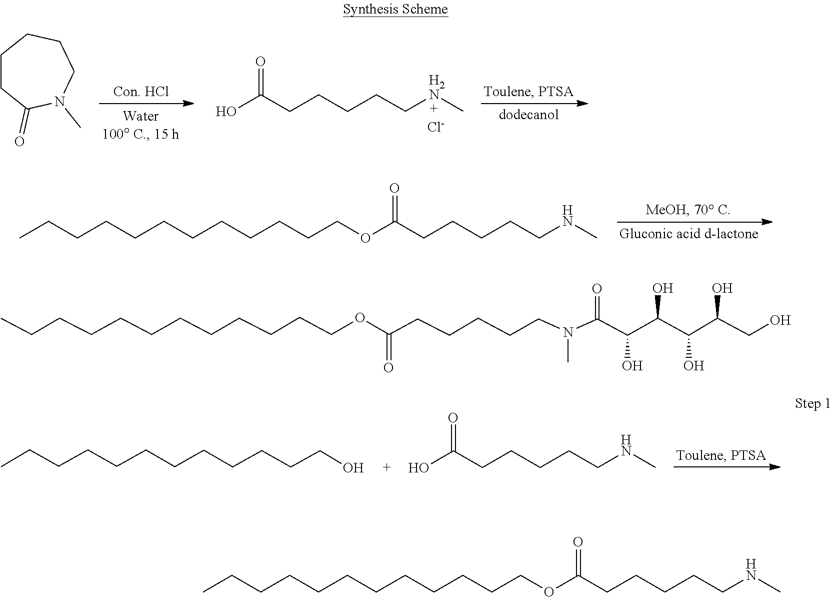

Step 1 synthesis procedure: 6-amino methyl hexanoic acid (1 equiv) followed by PTSA (1 equiv) were added to 12.8 g of dodecanol (1 equiv) in 20 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 16 g of brown liquid was obtained. (Giving a yield of about 76%).

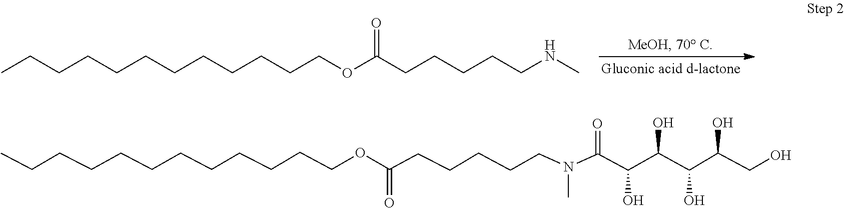

Step 2 synthesis procedure: Gluconic acid-lactone (1 equiv) was added to 1 g of amino methyl hexanoic ester (1 equiv) in 20 mL of MeOH at room temperature and then stirred for 14 h. The reaction mixture was then concentrated, and crude compound was poured into ice water. The yellow solid precipitate was filtered, dried under high vacuum. 0.6 g of yellow solid was obtained. (Giving a yield of about 37%).

### Example 21a: Determination of Physical Properties of Surfactant 21

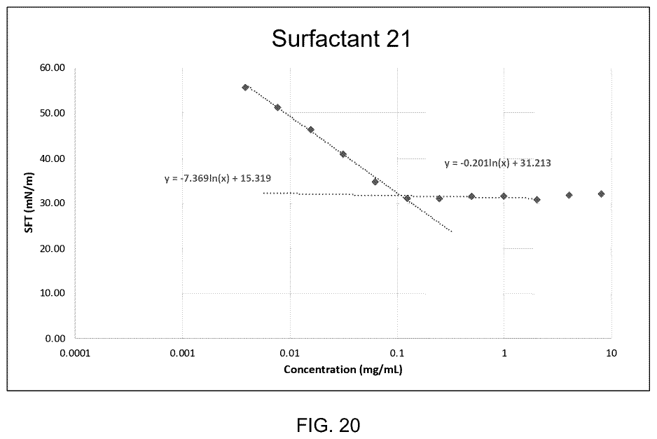

The critical micelle concentration (CMC) for Surfactant 21 was measured. From the surface tension change with concentration in water, the CMC was determined to be about 0.2349 mM (0.1089 mg/mL) at pH 4. The plateau value of minimum surface tension that can be reached by this surfactant was about 31.28 mN/m, indicating that the surfactant has outstanding interfacial activity. These results were plotted as surface tension versus concentration in FIG. 20.

The molecular parameters of surfactant 21 are summarized in Table 21:

TABLE 21

| | |
|---|---|
| CMC (mg/mL) | 0.1089 |
| CMC (mM) | 0.2349 |
| γ_{cmc} (mN/m) | 31.28 |
| 10³ × Γₘₐₓ (mmol/m²) | 3.02 |
| Åₘᵢₙ (Å²) | 54.92 |
| pH @ 8 mg/mL | 4.40 |
| pH @ 0.0039 mg/mL | 5.82 |
| prefactor? | 1 |

### Example 22: Synthesis of Surfactant 22

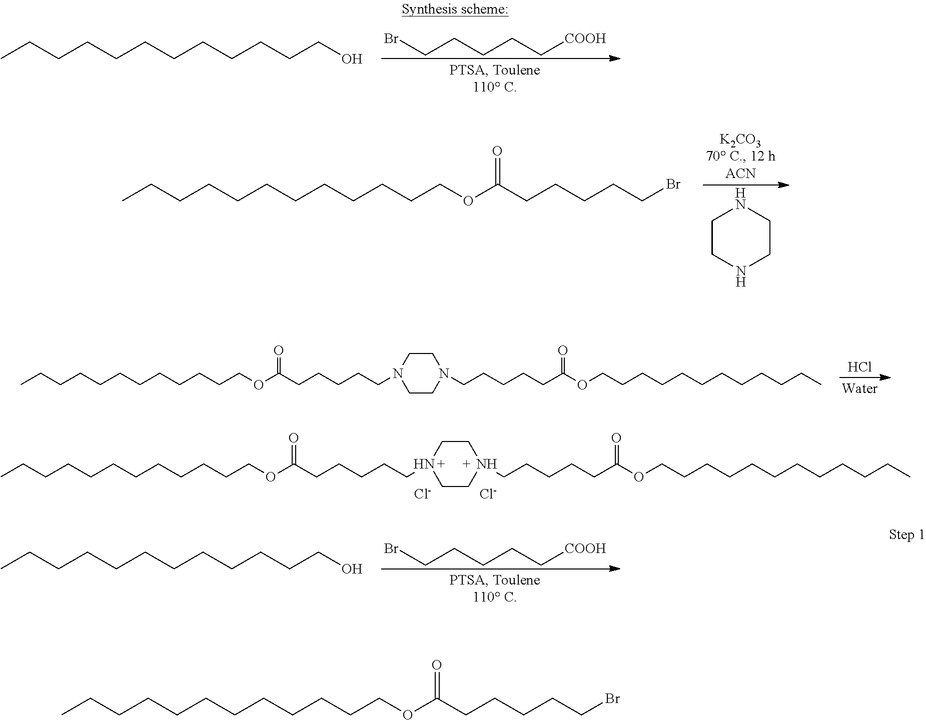

Step 1 synthesis procedure: Bromo hexanoic acid (1 equiv) followed by PTSA (1 equiv) were added to 1 g of dodecanol (1 equiv) in 20 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 1.8 g of yellow color liquid was obtained. (Giving a yield of about 90%).

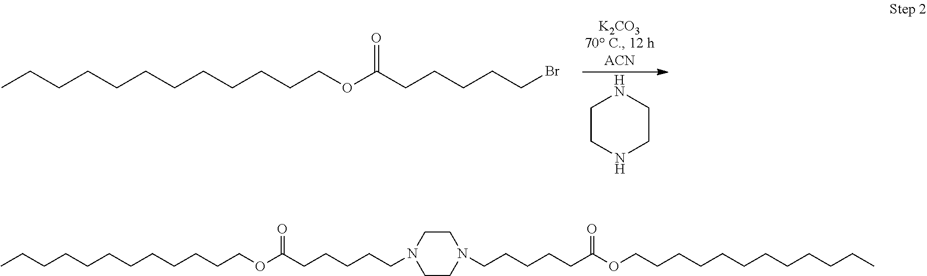 [SMILES: CCCCCCCCCCCCOC(=O)CCCCCBr ; C1CNCCN1 ; CCCCCCCCCCCCOC(=O)CCCCCN1CCN(CCCCCC(=O)OCCCCCCCCCCCC)CC1]

Step 2 synthesis procedure: Piperazine (0.5 equiv) followed by K₂CO₃ (1 equiv) were added to 0.6 g of bromo hexanoic ester (1 equiv) in 20 mL of ACN at room temperature and then heated at 70° C. for 12 h. The reaction mixture was filtered, then filtrate was concentrated, further crude compound was washed with hexanes to remove unreacted starting materials. 1 g of light brown liquid was obtained. (Giving a yield of about 95%).

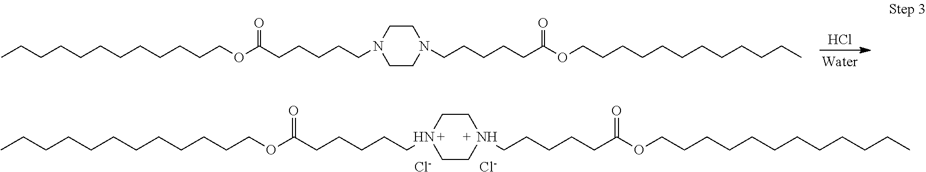

Step 3 synthesis procedure: HCl (2 equiv) was added to 0.6 g of piperazine hexanoic ester (1 equiv) in 10 mL of water at room temperature and then stirred for 3 h. The reaction mixture was concentrated, crude compound was washed with hexanes to remove unreacted starting materials. 0.55 g of white solid was obtained. (Giving a yield of about 83%).

### Example 22a: Determination of Physical Properties of Surfactant 22

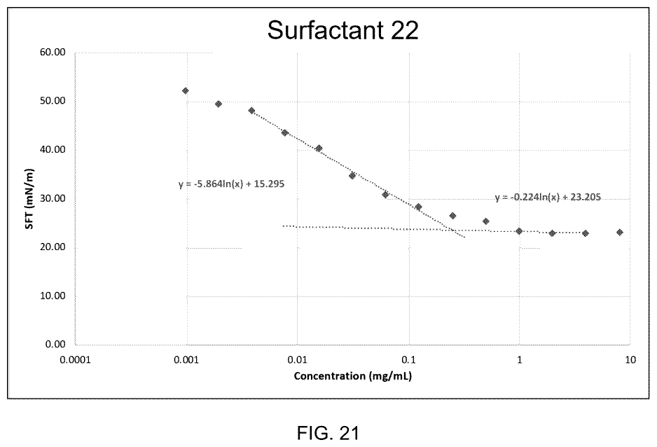

The critical micelle concentration (CMC) for Surfactant 22 was measured. From the surface tension change with concentration in water, the CMC was determined to be about 0.3398 mM (0.2460 mg/mL) at pH 3. The plateau value of minimum surface tension that can be reached by this surfactant was about 23.05 mN/m, indicating that the surfactant has outstanding interfacial activity. These results were plotted as surface tension versus concentration in FIG. 21.

The molecular parameters of surfactant 22 are summarized in Table 22:

TABLE 22

| | |
|---|---|
| CMC (mg/mL) | 0.2460 |
| CMC (mM) | 0.3398 |
| γ_{cmc} (mN/m) | 23.05 |
| 10³ × Γₘₐₓ (mmol/m²) | 1.20 |
| Åₘᵢₙ (Å²) | 138.04 |
| pH @ 8 mg/mL | 2.55 |
| pH @ 0.0097 mg/mL | 5.34 |
| prefactor? | 2 |

### Example 23: Synthesis of Surfactant 23

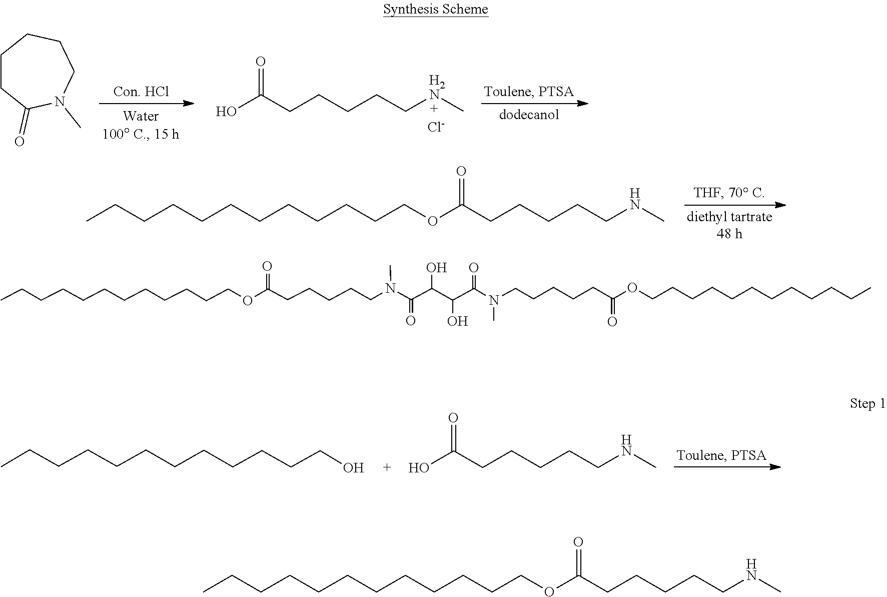

Step 1 synthesis procedure: 6-amino methyl hexanoic acid (1 equiv) followed by PTSA (1 equiv) were added to 12.8 g of dodecanol (1 equiv) in 20 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 16 g of brown liquid was obtained. (Giving a yield of about 76%).

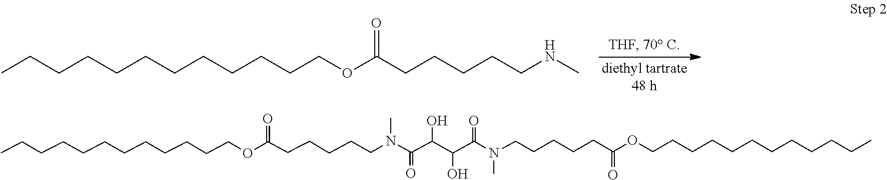

Step 2 synthesis procedure: Diethyl tartrate (0.5 equiv) was added to 1 g of amino methyl hexanoic ester (1 equiv) in 20 mL of THF at room temperature and then heated at 70° C. for 14 h. The reaction mixture was then concentrated, and crude compound was washed with ether, further compound was dried under high vacuum. 1.3 g of brown liquid was obtained. (Giving a yield of about 55%).

### Example 23a: Determination of Physical Properties of Surfactant 23

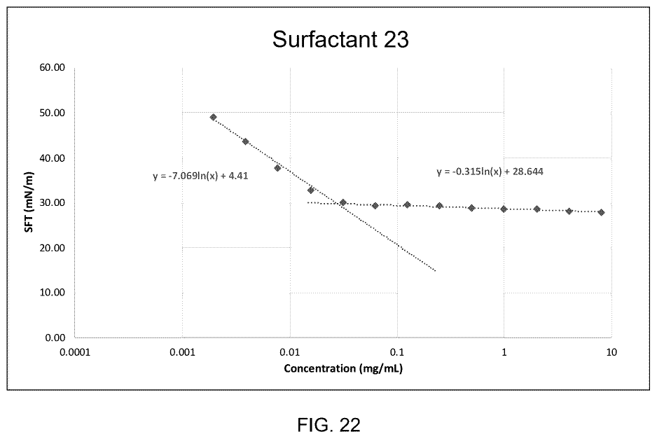

The critical micelle concentration (CMC) for Surfactant 23 was measured. From the surface tension change with concentration in water, the CMC was determined to be about 0.0404 mM (0.0277 mg/mL) at pH 8. The plateau value of minimum surface tension that can be reached by this surfactant was about 28.43 mN/m, indicating that the surfactant has outstanding interfacial activity. These results were plotted as surface tension versus concentration in FIG. 22.

The molecular parameters of surfactant 23 are summarized in Table 23:

TABLE 23

| | |
|---|---|
| CMC (mg/mL) | 0.0277 |
| CMC (mM) | 0.0404 |
| γ_{cmc} (mN/m) | 28.43 |
| 10³ × Γₘₐₓ (mmol/m²) | 2.90 |
| Åₘᵢₙ (Å²) | 57.25 |
| pH @ 8 mg/mL | 8.29 |
| pH @ 0.0019 mg/mL | 7.27 |
| prefactor? | 1 |

### Example 24: Synthesis of Surfactant 24

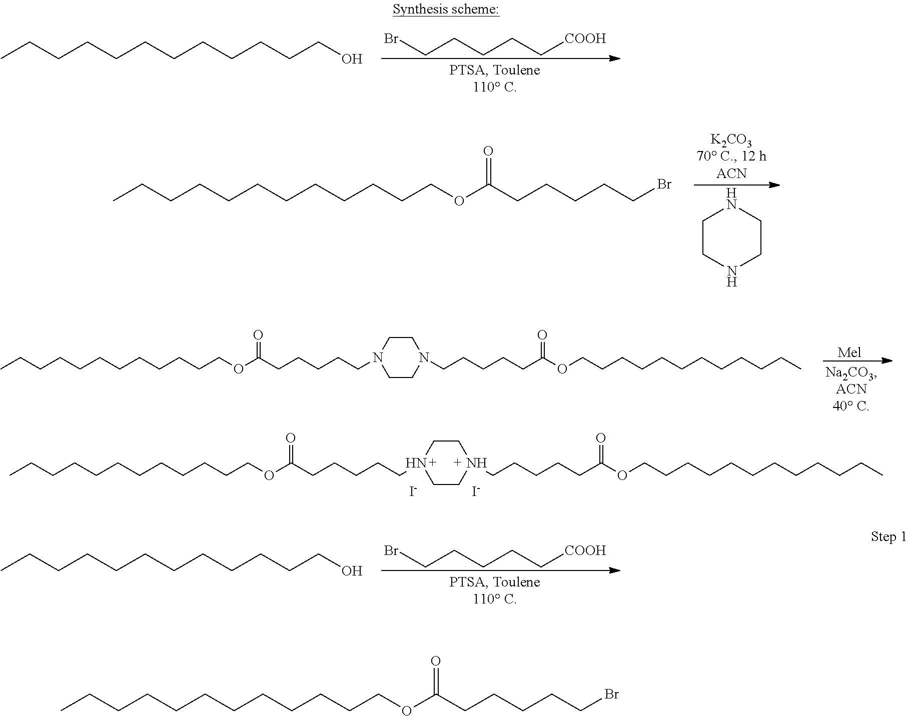

Step 1 synthesis procedure: Bromo hexanoic acid (1 equiv) followed by PTSA (1 equiv) were added to 1 g of dodecanol (1 equiv) in 20 mL of toulene at room temperature and then heated at 110° C. for 14 h. The reaction mixture was then diluted with DCM and washed with a Sat. NaHCO₃ solution (50 mL) followed by water (50 mL). The organic layer concentrated, and crude compound used for next step without any further purification. 1.8 g of yellow color liquid was obtained. (Giving a yield of about 90%).

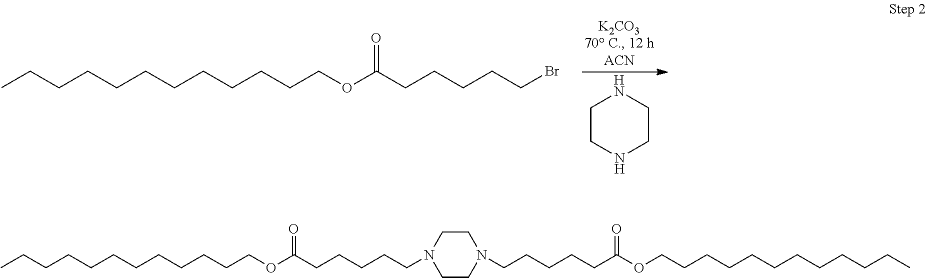 [SMILES: CCCCCCCCCCCCOC(=O)CCCCCBr ; C1CNCCN1 ; CCCCCCCCCCCCOC(=O)CCCCCN1CCN(CCCCCC(=O)OCCCCCCCCCCCC)CC1]

Step 2 synthesis procedure: Piperazine (0.5 equiv) followed by K₂CO₃ (1 equiv) were added to 0.6 g of bromo hexanoic ester (1 equiv) in 20 mL of ACN at room temperature and then heated at 70° C. for 12 h. The reaction mixture was filtered, then filtrate was concentrated, further crude compound was washed with hexanes to remove unreacted starting materials. 1 g of light brown liquid was obtained. (Giving a yield of about 95%).

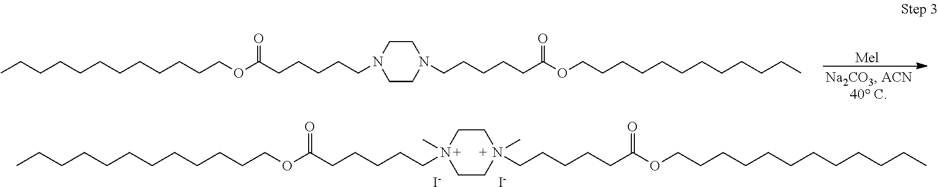

Step 3 synthesis procedure: Mel (2 equiv) followed by sodium carbonate (2 equiv) were added to 1 g of piperazine hexanoic ester (1 equiv) in 20 mL of ACN at room temperature and then stirred for 12 h at 40° C. The reaction mixture was filtered, and filtrate was concentrated, crude compound was washed with hexanes to remove unreacted starting materials. 1.3 g of white solid was obtained. (Giving a yield of about 90%).

### Example 24a: Determination of Physical Properties of Surfactant 24

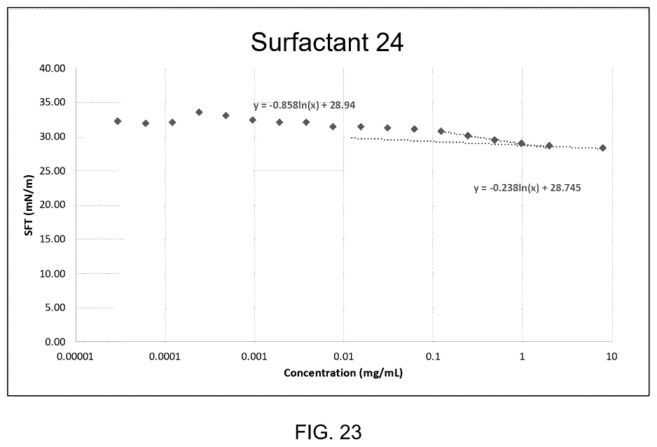

The critical micelle concentration (CMC) for Surfactant 24 was measured. From the surface tension change with concentration in water, the CMC was determined to be about 0.9089 mM (0.8370 mg/mL) at pH 6. The plateau value of minimum surface tension that can be reached by this surfactant was about 28.42 mN/m, indicating that the surfactant has outstanding interfacial activity. These results were plotted as surface tension versus concentration in FIG. 23.

The molecular parameters of surfactant 24 are summarized in Table 24:

TABLE 24

| | |
|---|---|
| CMC (mg/mL) | 0.8370 |
| CMC (mM) | 0.9089 |
| γ_{cmc} (mN/m) | 28.42 |
| 10³ × Γₘₐₓ (mmol/m²) | 0.09 |
| Åₘᵢₙ (Å²) | 1886.83 |
| pH @ 8 mg/mL | 6.27 |
| pH @ 0.00006 mg/mL | 6.55 |
| prefactor? | 4 |

### Aspects

The subject matter encompassed by the following numbered aspects also forms part of the present disclosure, optionally in combination with the subject matter described above and/or defined in the claims that follow.
Aspect 1 A compound of Formula I, wherein the compound of Formula I can be a salt, below:

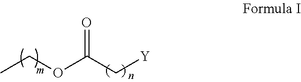

wherein Y is selected from formula (1-1), (1-2), or (1-3)

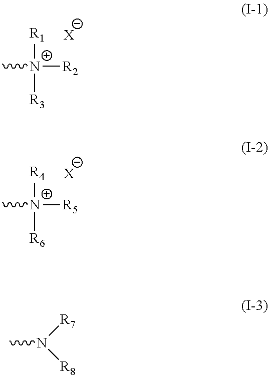

the wave-line denoting the connection to Formula I;
   wherein
R₁ and R₂ are each one of C₁-C₁₀ alkyl and are linked or otherwise connected together to form a 4 to 20 membered ring that may be substituted or unsubstituted, saturated or unsaturated; optionally the ring may include one or more of oxygen, nitrogen, or sulfur atoms or groups that include at least one of these atoms at any suitable position in the ring, and the ring may be optionally substituted with one or more substituents selected from the group consisting of one of C₁-C₆ alkyl, hydroxyl, amino, amido, sulfonyl, sulfonate, carbonyl, carboxyl, and carboxylate; wherein the ring formed from R₁ and R₂ is optionally linked and/or fused to a further ring;
R₃ is selected from the group consisting of hydrogen, one of C₁-C₆ alkyl, C₂-C₁₀ alkenyl, C₂-C₁₀ alkynyl, C₂-C₁₂ ester, hydroxyl, C₁-C₁₀ hydroxyl, aryl alkyl, C₂-C₁₂ alkoxy alkyl ether, alkyl phosphate, C₃-C₈ carboxylic acid, C₁-C₁₀ alkyl benzoic acid, oxygen, and sulfur;
R₄ and R₅ may be the same or different, and are individually selected from the group consisting of hydrogen and one of C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate;
R₆ is and is selected from the group consisting of C₂-C₁₀ alkenyl, C₂-C₁₀ alkynyl, C₂-C₁₂ ester, hydroxyl, aryl alkyl, C₂-C₁₂ alkoxy alkyl ether, alkyl phosphate, C₃-C₈ carboxylic acid, C₁-C₁₀ alkyl benzoic acid, amino, amido, and sulfur; wherein R₆ may optionally be substituted with one or more substituents selected from the group consisting of aryl, heteroaryl, hydroxyl, amino, amido, sulfonyl, sulfonate, carbonyl, carboxyl, and carboxylate;
R₇ and R₈ may be the same or different, and are individually selected from the group consisting of hydrogen and C₁-C₆ alkyl, wherein the C₁-C₆ alkyl is substituted with one or more substituents selected from the group consisting of hydroxyl, amino, amido, sulfonyl, sulfonate, carbonyl, carboxyl, and carboxylate, or a salt thereof, wherein a counterion W associated with any of the hydroxyl, sulfonate, or carboxylate group is selected from Na, K or Ca;
X is selected from the group consisting of chloride, bromide, and iodide.
n is an integer in the range of 2 to 5; and
m is an integer in the range of 9 to 20.
Aspect 2 is the compound of Aspect 1, wherein R₄ and R₅ are each individually one of C₁-C₆ alkyl.
Aspect 3 is the compound of Aspect 1, wherein R₄ and R₅ are both methyl.
Aspect 4 is the compound of Aspect 1, wherein R₄ is hydrogen and R₅ is methyl.
Aspect 5 is the compound of any one of Aspects 1 to 4, wherein R₆ is one of C₂-C₁₀ alkenyl, C₂-C₁₀ alkynyl, C₂-C₁₂ ester, aryl alkyl, or C₂-C₁₂ alkoxy alkyl ether.
Aspect 6 is the compound of any one of Aspects 1 to 5, wherein R₇ is methyl.
Aspect 7 is the compound of any one of Aspects 1 to 5, wherein R₇ is hydrogen.
Aspect 8 is the compound of any one of Aspects 1 to 7, wherein R₈ is a C₆ alkyl.
Aspect 9 is the compound of any one of Aspects 1 to 7, wherein R₈ is a C₅ alkyl.
Aspect 10 is the compound of any one of Aspects 1 to 7, wherein R₈ is a C₄ alkyl.
Aspect 11 is the compound of any one of Aspects 1 to 10, wherein Y is selected from piperidine, pyridine, pyrimidine, pyrrole, imidazole, pyrazole, piperazine, or morpholine.
Aspect 12 is the compound of Aspect 1 or 11, wherein Y is

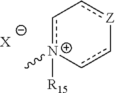

wherein:
R₁₅ is either not present, or an oxygen, hydrogen, one of C₁-C₆ alkyl, or one of C₁-C₆ heteroalkyl;
Z is 0, S, NR₁₆R₁₇, or CR₁₈R₁₉
n is an integer in the range of 2 to 5 and m is an integer in the range of 9 to 20;
X is selected from the group consisting of chloride, bromide, and iodide; and
R₁₆ and R₁₇ are each independently a hydrogen or one of C₁-C₆ alkyl;
R₁₈ and R₁₉ are each independently either not present, or a hydrogen, or one of C₁-C₆ alkyl;
[symbol] are single bonds or double bonds; and
wherein the ring may be optionally substituted with one or more substituents selected from the group consisting of C₁-C₆ alkyl, hydroxyl, amino, amido, sulfonyl, sulfonate, carbonyl, carboxyl, and carboxylate.
Aspect 13 is the compound of any one of Aspects 1 to 12, wherein R₃ and/or R₁₅ is oxygen.
Aspect 14 is the compound of any one of Aspects 1 to 12, wherein R₃ and/or R₁₅ is hydrogen.
Aspect 15 is the compound of any one of Aspects 1 to 12, wherein R₃ and/or R₁₅ is C₁-C₆ alkyl or C₁-C₆ heteroalkyl.
Aspect 16 is the compound of any one of Aspects 1 to 12, wherein R₃ and/or R₁₅ is methyl.
Aspect 17 is the compound of any one of Aspects 1 to 12, wherein R₃ and/or R₁₅ is C₁-C₆ alkyl substituted with hydroxy.
Aspect 18 is the compound of any one of Aspects 12 to 17, wherein R₁₇ is methyl.
Aspect 19 is the compound of any one of Aspects 12 to 18, wherein R₁₆ is methyl.
Aspect 20 is the compound of any one of Aspects 12 to 18, wherein R₁₆ is hydrogen.
Aspect 21 is the compound of any one of Aspects 12 to 17, wherein R₁₉ is hydrogen.
Aspect 22 is the compound of any one of Aspects 12 to 17, wherein R₁₉ is not present.
Aspect 23 is the compound of any one of Aspects 12 to 17, wherein R₁₈ is hydrogen.
Aspect 24 is the compound of any one of Aspects 12 to 17, wherein R₁₈ is C₁-C₆ alkyl.
Aspect 25 is the compound of any one of Aspects 12 to 17, wherein R₁₈ is not present.
Aspect 26 is the compound of any one of Aspects 1 to 25, wherein X is chloride.
Aspect 27 is the compound of any one of Aspects 1 to 25, wherein X is iodide.
Aspect 28 is the compound of any one of Aspects 1 to 25, wherein X is bromide.
Aspect 29 is the compound of Aspect 1, wherein Y is

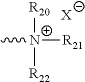

R₂₀ and R₂₁ are each independently methyl or hydrogen;
n is an integer in the range of 2 to 5 and m is an integer in the range of 9 to 20;
X is selected from the group consisting of chloride, bromide, and iodide;
R₂₂ is selected from the group consisting of C₂-C₁₀alkenyl, C₂-C₁₀alkynyl, C₂-C₁₂ ester, hydroxyl, aryl C₁-C₆ alkyl, C₂-C₁₂ alkoxy alkyl ether, C₁-C₆ alkyl phosphate, C₃-C₈ carboxylic acid, C₁-C₁₀alkyl benzoic acid, amino, amido, and sulfur; wherein R₂₂ may optionally be substituted with one or more substituents selected from the group consisting of aryl, heteroaryl, hydroxyl, amino, amido, sulfonyl, sulfonate, carbonyl, carboxyl, and carboxylate.
Aspect 30 is the compound of any one of Aspects 1-29, wherein Y is selected from:

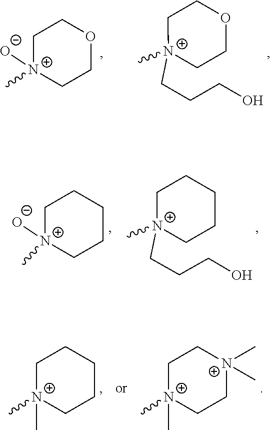

Aspect 31 is the compound of any one of Aspects 1-29, wherein Y is selected from:

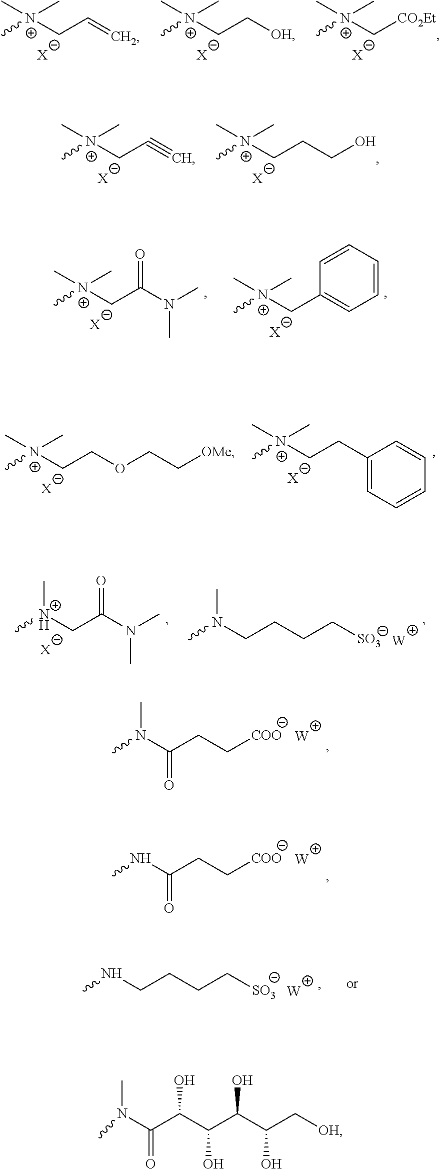

wherein
X is selected from the group consisting of chloride, bromide, and iodide, and
W is selected from the group consisting of sodium, potassium, or calcium.
Aspect 32 is the compound of any one of Aspects 1 to 31, wherein n is 3, 4, 5, 6, 7, 8, 9, 10, 11 or 12.
Aspect 33 is the compound of any one of Aspects 1 to 32, wherein n is 5.
Aspect 34 is the compound of any one of Aspects 1 to 33, wherein m is 9, 10, 11, 12, 13, 14, 15, 16, 17, 18, 19, or 20.
Aspect 35 is the compound of any one of Aspects 1 to 34, wherein m is 11.
Aspect 36 is the compound of any one of Aspects 1 to 35, wherein the counterion is present and is selected from the group consisting of chloride, bromide, and iodide.
Aspect 37 is the compound of any one of Aspects 1 to 35, wherein the counterion is chloride.
Aspect 38 is the compound of any one of Aspects 1 to 35, wherein the counterion is iodide.
Aspect 39 is the compound of any one of Aspects 1 to 35, wherein the counterion is bromide.
Aspect 40 is the compound of Aspect 1, wherein the compound is 1-(6-(dodecyloxy)-6-oxohexyl)pyridin-1-ium bromide:

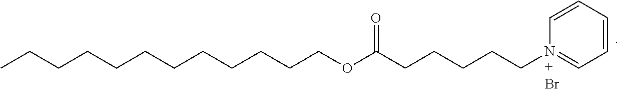

Aspect 41 is the compound of Aspect 1, wherein the compound is 4-(6-(dodecyloxy)-6-oxohexyl)-4-(3-hydroxypropyl)morpholin-4-ium iodide:

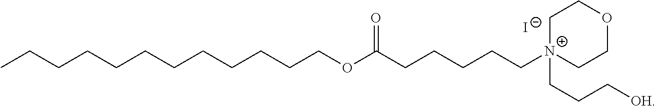 [SMILES: CCCCCCCCCCCCOC(=O)CCCCC[N+]1(CCCO)CCOCC1 ; [I-]]

Aspect 42 is the compound of Aspect 1, wherein the compound is 1-(6-(dodecyloxy)-6-oxohexyl)piperidin-1-ium chloride:

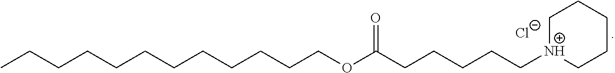 [SMILES: CCCCCCCCCCCCOC(=O)CCCCC[NH+]1CCCCC1 ; [Cl-]]

Aspect 43 is the compound of Aspect 1, wherein the compound is 4-(6-(dodecyloxy)-6-oxohexyl)morpholin-4-ium chloride:

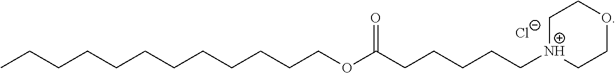 [SMILES: CCCCCCCCCCCCOC(=O)CCCCC[NH+]1CCOCC1 ; [Cl-]]

Aspect 44 is the compound of Aspect 1, wherein the compound is 1-(6-(dodecyloxy)-6-oxohexyl)-4-methylpiperazine-1,4-diium dichloride:

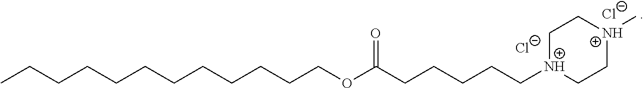 [SMILES: [Cl-] ; CCCCCCCCCCCCOC(=O)CCCCC[NH+]1CC[NH+](C)CC1 ; [Cl-]]

Aspect 45 is the compound of Aspect 1, wherein the compound is 4-(6-(dodecyloxy)-6-oxohexyl)morpholine 4-oxide:

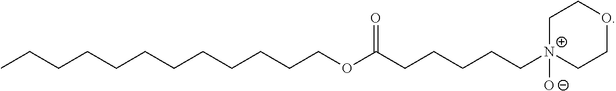 [SMILES: CCCCCCCCCCCCOC(=O)CCCCC[N+]1([O-])CCOCC1]

Aspect 46 is the compound of Aspect 1, wherein the compound is N-allyl-6-(dodecyloxy)-N,N-dimethyl-6-oxohexan-1-aminium iodide:

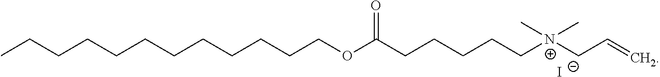 [SMILES: C=CC[N+](C)(C)CCCCCC(=O)OCCCCCCCCCCCC ; [I-]]

Aspect 47 is the compound of Aspect 1, wherein the compound is 6-(dodecyloxy)-N-(2-hydroxyethyl)-N,N-dimethyl-6-oxohexan-1-aminium iodide:

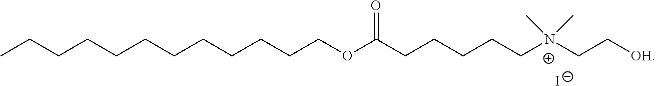 [SMILES: CCCCCCCCCCCCOC(=O)CCCCC[N+](C)(C)CCO ; [I-]]

Aspect 48 is the compound of Aspect 1, wherein the compound is 6-(dodecyloxy)-N-(2-ethoxy-2-oxoethyl)-N,N-dimethyl-6-oxohexan-1-aminium bromide:

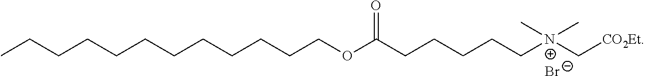 [SMILES: CCCCCCCCCCCCOC(=O)CCCCC[N+](C)(C)CC(=O)OCC ; [Br-]]

Aspect 49 is the compound of Aspect 1, wherein the compound is 6-(dodecyloxy)-N,N-dimethyl-6-oxo-N-(prop-2-yn-1-yl)hexan-1-aminium bromide:

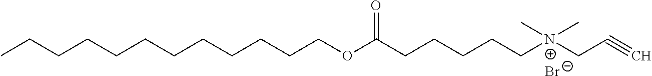 [SMILES: C#CC[N+](C)(C)CCCCCC(=O)OCCCCCCCCCCCC ; [Br-]]

Aspect 50 is the compound of Aspect 1, wherein the compound is 6-(dodecyloxy)-N-(3-hydroxypropyl)-N,N-dimethyl-6-oxohexan-1-aminium iodide:

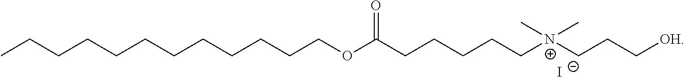 [SMILES: CCCCCCCCCCCCOC(=O)CCCCC[N+](C)(C)CCCO ; [I-]]

Aspect 51 is the compound of Aspect 1, wherein the compound is N-(2-(dimethylamino)-2-oxoethyl)-6-(dodecyloxy)-N,N-dimethyl-6-oxohexan-1-aminium iodide:

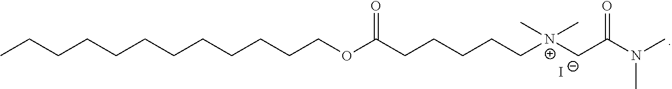 [SMILES: CCCCCCCCCCCCOC(=O)CCCCC[N+](C)(C)CC(=O)N(C)C ; [I-]]

Aspect 52 is the compound of Aspect 1, wherein the compound is N-benzyl-6-(dodecyloxy)-N,N-dimethyl-6-oxohexan-1-aminium bromide:

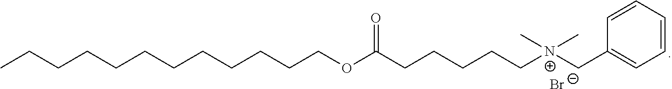 [SMILES: CCCCCCCCCCCCOC(=O)CCCCC[N+](C)(C)Cc1ccccc1 ; [Br-]]

Aspect 53 is the compound of Aspect 1, wherein the compound is 6-(dodecyloxy)-N-(2-(2-methoxyethoxy)ethyl)-N,N-dimethyl-6-oxohexan-1-aminium bromide:

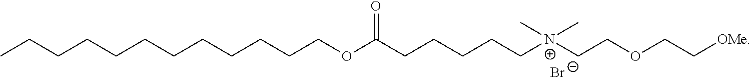 [SMILES: CCCCCCCCCCCCOC(=O)CCCCC[N+](C)(C)CCOCCOC ; [Br-]]

Aspect 54 is the compound of Aspect 1, wherein the compound is 6-(dodecyloxy)-N,N-dimethyl-6-oxo-N-phenethylhexan-1-aminium iodide:

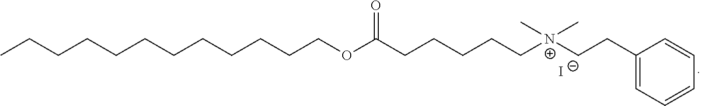 [SMILES: CCCCCCCCCCCCOC(=O)CCCCC[N+](C)(C)CCc1ccccc1 ; [I-]]

Aspect 55 is the compound of Aspect 1, wherein the compound is N-(2-(dimethylamino)-2-oxoethyl)-6-(dodecyloxy)-N-methyl-6-oxohexan-1-aminium chloride:

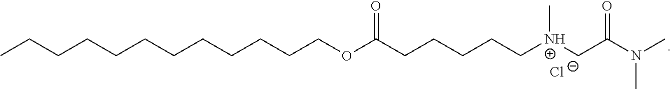 [SMILES: CCCCCCCCCCCCOC(=O)CCCCC[NH+](C)CC(=O)N(C)C ; [Cl-]]

Aspect 56 is the compound of Aspect 1, wherein the compound is sodium 4-((6-(dodecyloxy)-6-oxohexyl)(methyl)amino)butane-1-sulfonate:

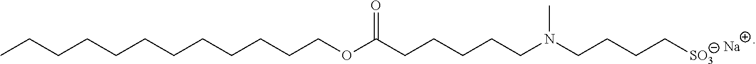

Aspect 57 is the compound of Aspect 1, wherein the compound is sodium 5-((6-(dodecyloxy)-6-oxohexyl)(methyl)amino)pentanoate:

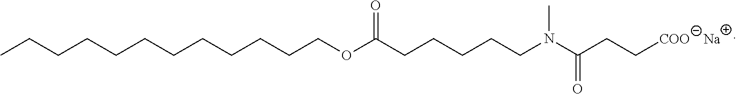

Aspect 58 is the compound of Aspect 1, wherein the compound is sodium 5-((6-(dodecyloxy)-6-oxohexyl)amino)pentanoate:

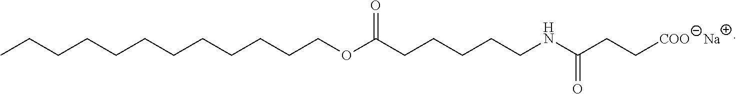

Aspect 59 is the compound of Aspect 1, wherein the compound is sodium 4-((6-(dodecyloxy)-6-oxohexyl)amino)butane-1-sulfonate:

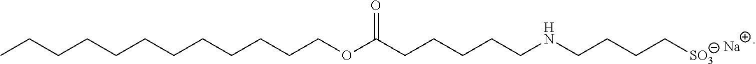

Aspect 60 is the compound of Aspect 1, wherein the compound is dodecyl 6-(2,3,4,5,6-pentahydroxy-N-methylhexanamido)hexanoate:

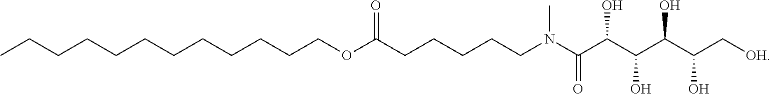 [SMILES: CCCCCCCCCCCCOC(=O)CCCCCN(C)C(=O)[C@H](O)[C@@H](O)[C@@H](O)[C@@H](O)CO]

Aspect 61 is a surfactant of Formula (I) as defined in any one of Aspects 1 to 59.
Aspect 62 is the use of a compound of Formula (I) as defined in any one of Aspects 1 to 59 as a surfactant.
Aspect 63 is a compound of Formula (II), wherein the compound of Formula II can be a salt, below:

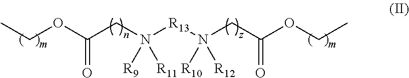

R₉, R₁₀, may be the same or different, and are selected from the group consisting of hydrogen and C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate, optionally, R₉ and R₁₀ are linked or otherwise connected together to form a ring that may be substituted or unsubstituted, saturated or unsaturated; optionally the ring may include one or more of oxygen, nitrogen, or sulfur atoms or groups that include at least one of these atoms at any suitable position on the ring, and the ring may be optionally substituted with one or more substituents selected from the group consisting of any one of C₁-C₆ alkyl, hydroxyl, amino, amido, sulfonyl, sulfonate, carbonyl, carboxyl, and carboxylate, wherein the ring formed from R₉ is optionally linked and/or fused to a further ring;
R₁₁, R₁₂ are optionally present, and if present are the same or different, and are individually selected from the group consisting of hydrogen and C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate;
if R₁₁ and R₁₂ are present, a counter ion Z is selected from the group consisting of chloride, bromide, and iodide.
R₁₃ is selected from the group consisting of C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate;
Z is selected from the group consisting of chloride, bromide, and iodide.
n and z are, independently, an integer in the range of 2 to 5; and
m is an integer in the range of 9 to 20.
Aspect 64 is the compound of Aspect 63, wherein R₉ and R₁₀ are linked or otherwise connected together to form a ring that may be substituted or unsubstituted, saturated or unsaturated; optionally the ring may include one or more of oxygen, nitrogen, or sulfur atoms or groups that include at least one of these atoms at any suitable position on the ring, and the ring may be optionally substituted with one or more substituents selected from the group consisting of one of C₁-C₆ alkyl, hydroxyl, amino, amido, sulfonyl, sulfonate, carbonyl, carboxyl, and carboxylate, wherein the ring formed from R₉ is optionally linked and/or fused to a further ring.
Aspect 65 is the compound of Aspect 63 or 64, wherein n is 5 and m is 11.
Aspect 66 is the compound of any one of Aspects 63 to 65, wherein Z is chloride or iodide.
Aspect 67 is the compound of any one of Aspects 63 to 66, wherein R₉ is methyl.
Aspect 68 is the compound of any one of Aspects 63 to 67, wherein R₁₁ is not present.
Aspect 69 is the compound of any one of Aspects 63 to 68, wherein R₁₀ is methyl.
Aspect 70 is the compound of any one of Aspects 63 to 69, wherein R₁₂ is not present.
Aspect 71 is the compound of Aspect 63, wherein the compound is 1,4-bis(6-(dodecyloxy)-6-oxohexyl)piperazine-1,4-diium dichloride:

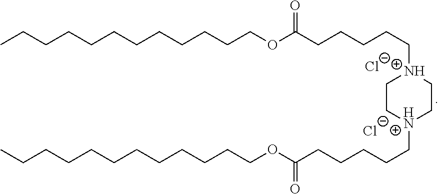

Aspect 72 is the compound of Aspect 63, wherein the compound is didodecyl 6,6′-((2,3-dihydroxysuccinyl)bis(methylazanediyl))dihexanoate:

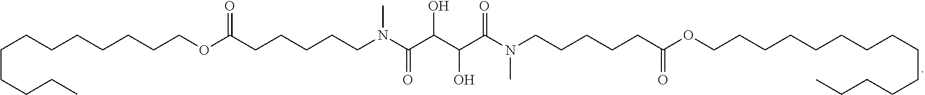 [SMILES: CCCCCCCCCCCCCCOC(=O)CCCCCN(C)C(=O)C(O)C(O)C(=O)N(C)CCCCCC(=O)OCCCCCCCCCCCC]

Aspect 73 is the compound of Aspect 63, wherein the compound is 1,4-bis(6-(dodecyloxy)-6-oxohexyl)-1,4-dimethylpiperazine-1,4-diium diiodide:

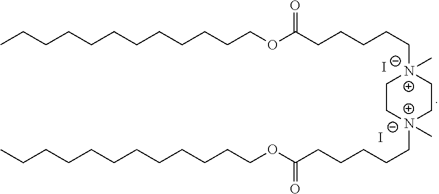

Aspect 74 is the compound of Aspect 63, wherein the compound of Formula (II) is represented as:

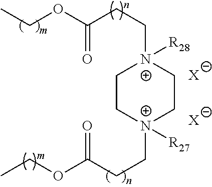

wherein
R₂₇ and R₂₈ are each independently a hydrogen or one of C₁-C₆ alkyl;
n is an integer in the range of 1 to 4;
m is an integer in the range of 9 to 20; and
X is selected from the group consisting of chloride, bromide, and iodide.
Aspect 75 is the compound of Aspect 74, wherein R₂₇ and R₂₈ are individually one of C₁-C₆ alkyl.
Aspect 76 is the compound of Aspect 74, wherein R₂₇ and R₂₈ are hydrogen.
Aspect 77 is the compound of any one of Aspects 74 to 76, wherein each n is 4.
Aspect 78 is the compound of any one of Aspects 74 to 77, wherein each m is 11.
Aspect 79 is the compound of any one of Aspects 74 to 78, wherein each X is iodide.
Aspect 80 is the compound of any one of Aspects 74 to 78, wherein each X is chloride.
Aspect 81 is the compound of any one of Aspects 74 to 78, wherein each X is bromide.
Aspect 82 is a surfactant of Formula (II) as defined in any one of Aspects 63 to 81.
Aspect 83 is the use of a compound of Formula (II) as defined in any one of Aspects 63 to 81 as a surfactant.

### Features

Exemplary features of this invention are captured below:

Feature 1 is a compound of Formula I, wherein the compound of Formula I can be a salt, below:

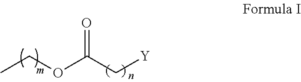

wherein Y is selected from formula (1-1), (1-2), or (1-3)

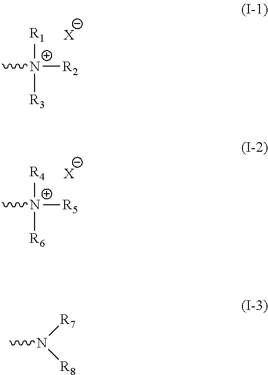

the wave-line denoting the connection to Formula I;
wherein
R₁ and R₂ are each one of C₁-C₁₀ alkyl and are linked or otherwise connected together to form a 4 to 20 membered ring that may be substituted or unsubstituted, saturated or unsaturated; optionally the ring may include one or more of oxygen, nitrogen, or sulfur atoms or groups that include at least one of these atoms at any suitable position in the ring, and the ring may be optionally substituted with one or more substituents selected from the group consisting of one of C₁-C₆ alkyl, hydroxyl, amino, amido, sulfonyl, sulfonate, carbonyl, carboxyl, and carboxylate; wherein the ring formed from R₁ and R₂ is optionally linked and/or fused to a further ring;
R₃ is selected from the group consisting of hydrogen, C₁-C₆ alkyl, C₂-C₁₀ alkenyl, C₂-C₁₀ alkynyl, C₂-C₁₂ ester, hydroxyl, C₁-C₁₀ hydroxyl, aryl C₁-C₆ alkyl, C₂-C₁₂ alkoxy alkyl ether, C₁-C₆ alkyl phosphate, C₃-C₈ carboxylic acid, C₁-C₁₀ alkyl benzoic acid, oxygen, and sulfur;
R₄ and R₅ may be the same or different, and they are individually selected from the group consisting of hydrogen and C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate;
R₆ is and is selected from the group consisting of C₂-C₁₀ alkenyl, C₂-C₁₀ alkynyl, C₂-C₁₂ ester, hydroxyl, aryl C₁-C₆ alkyl, C₂-C₁₂ alkoxy alkyl ether, C₁-C₆ alkyl phosphate, C₃-C₈carboxylic acid, C₁-C₁₀alkyl benzoic acid, amino, amido, and sulfur; wherein R₆ may optionally be substituted with one or more substituents selected from the group consisting of aryl, heteroaryl, hydroxyl, amino, amido, sulfonyl, sulfonate, carbonyl, carboxyl, and carboxylate;
R₇ and R₈ may be the same or different, and are selected from the group consisting of hydrogen and C₁-C₆ alkyl, wherein the C₁-C₆ alkyl is substituted with one or more substituents selected from the group consisting of hydroxyl, amino, amido, sulfonyl, sulfonate, carbonyl, carboxyl, and carboxylate, or a salt thereof, wherein a counterion W associated with any of the hydroxyl, sulfonate, or carboxylate group is selected from Na, K or Ca;
X is selected from the group consisting of chloride, bromide, and iodide.
n is an integer in the range of 2 to 5; and
m is an integer in the range of 9 to 20.

Feature 2 is the compound of Feature 1, wherein Y is selected from piperidine, pyridine, pyrimidine, pyrrole, imidazole, pyrazole, piperazine, or morpholine.

Feature 3 is the compound of Feature 1, wherein Y is:

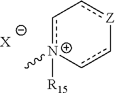

wherein:
R₁₅ is either not present, or an oxygen, hydrogen, one of C₁-C₆ alkyl, or one of C₁-C₆ heteroalkyl;
Z is 0, S, NR₁₆R₁₇, or CR₁₈R₁₉
n is an integer in the range of 2 to 5 and m is an integer in the range of 9 to 20;
X is selected from the group consisting of chloride, bromide, and iodide; and
R₁₆ and R₁₇ are each independently hydrogen or one of C₁-C₆ alkyl;
R₁₈ and R₁₉ are each independently not present, hydrogen, or one of C₁-C₆ alkyl;
[symbol] are single bonds or double bonds; and
wherein the ring may be optionally substituted with one or more substituents selected from the group consisting of one of C₁-C₆ alkyl, hydroxyl, amino, amido, sulfonyl, sulfonate, carbonyl, carboxyl, and carboxylate.

Feature 4 is the compound of Feature 1, wherein R₃ is oxygen.

Feature 5 is the compound of Feature 1, wherein R₃ is one of C₁-C₆ alkyl or one of C₁-C₆ heteroalkyl.

Feature 6 is the compound of Feature 1, wherein R₃ is one of C₁-C₆ alkyl substituted with hydroxy.

Feature 7 is the compound of Feature 3, wherein R₁₅ is oxygen.

Feature 8 is the compound of Feature 3, wherein R₁₅ is one of C₁-C₆ alkyl or one of C₁-C₆ heteroalkyl.

Feature 9 is the compound of Feature 1 wherein Y is

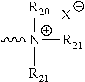

R₂₀ and R₂₁ are each independently methyl or a hydrogen;
n is an integer in the range of 2 to 5 and m is an integer in the range of 9 to 20;
X is selected from the group consisting of chloride, bromide, and iodide;
R₂₂ is selected from the group consisting of C₂-C₁₀alkenyl, C₂-C₁₀alkynyl, C₂-C₁₂ ester, hydroxyl, aryl C₁-C₆ alkyl, C₂-C₁₂ alkoxy alkyl ether, C₁-C₆ alkyl phosphate, C₃-C₈ carboxylic acid, C₁-C₁₀alkyl benzoic acid, amino, amido, and sulfur; wherein R₂₂ may optionally be substituted with one or more substituents selected from the group consisting of aryl, heteroaryl, hydroxyl, amino, amido, sulfonyl, sulfonate, carbonyl, carboxyl, and carboxylate.

Feature 10 is the compound of Feature 1, wherein Y is selected from:

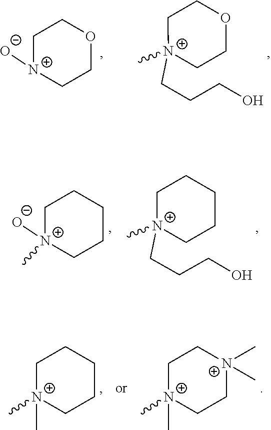

Feature 11 is the compound of Feature 1, wherein Y is selected from:

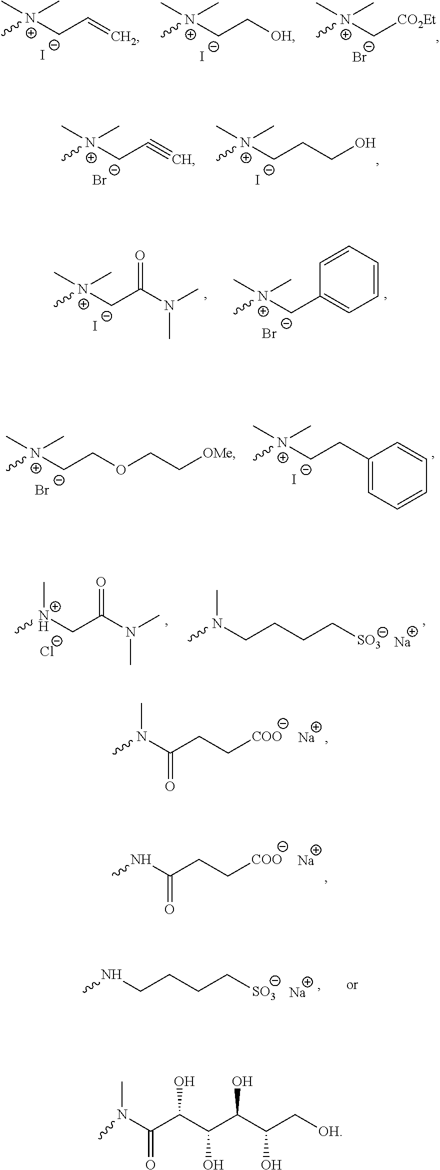

Feature 12 is the compound of Feature 1, wherein n is 3, 4, or 5.

Feature 13 is the compound of Feature 1, wherein n is 5.

Feature 14 is the compound of Feature 1, wherein the counterion is present and is selected from the group consisting of chloride, bromide, and iodide.

Feature 15 is the compound of Feature 1, wherein the counterion is chloride

Feature 16 is the compound of Feature 1, wherein the counterion is iodide.

Feature 17 is the compound of Feature 1, wherein the compound is 1-(6-(dodecyloxy)-6-oxohexyl)pyridin-1-ium bromide:

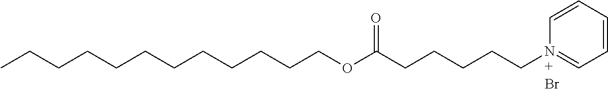

Feature 18 is the compound of Feature 1, wherein the compound is 4-(6-(dodecyloxy)-6-oxohexyl)-4-(3-hydroxypropyl)morpholin-4-ium iodide:

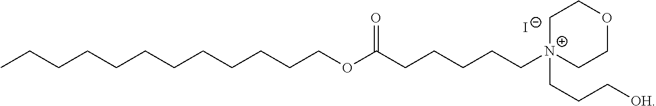 [SMILES: CCCCCCCCCCCCOC(=O)CCCCC[N+]1(CCCO)CCOCC1 ; [I-]]

Feature 19 is the compound of Feature 1, wherein the compound is 1-(6-(dodecyloxy)-6-oxohexyl)piperidin-1-ium chloride:

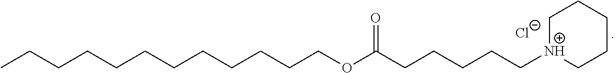 [SMILES: CCCCCCCCCCCCOC(=O)CCCCC[NH+]1CCCCC1 ; [Cl-]]

Feature 20 is the compound of Feature 1, wherein the compound is 4-(6-(dodecyloxy)-6-oxohexyl)morpholin-4-ium chloride:

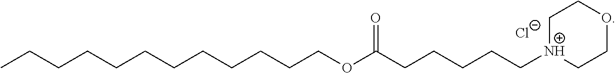 [SMILES: CCCCCCCCCCCCOC(=O)CCCCC[NH+]1CCOCC1 ; [Cl-]]

Feature 21 is the compound of Feature 1, wherein the compound is 1-(6-(dodecyloxy)-6-oxohexyl)-4-methylpiperazine-1,4-diium dichloride:

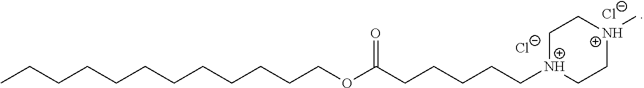 [SMILES: [Cl-] ; CCCCCCCCCCCCOC(=O)CCCCC[NH+]1CC[NH+](C)CC1 ; [Cl-]]

Feature 22 is the compound of Feature 1, wherein the compound is is 4-(6-(dodecyloxy)-6-oxohexyl)morpholine 4-oxide:

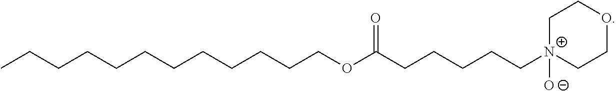 [SMILES: CCCCCCCCCCCCOC(=O)CCCCC[N+]1([O-])CCOCC1]

Feature 23 is the compound of Feature 1, wherein the compound is N-allyl-6-(dodecyloxy)-N,N-dimethyl-6-oxohexan-1-aminium iodide:

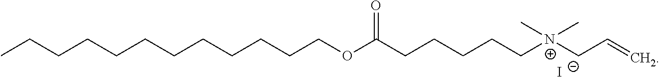 [SMILES: C=CC[N+](C)(C)CCCCCC(=O)OCCCCCCCCCCCC ; [I-]]

Feature 24 is the compound of Feature 1, wherein the compound is 6-(dodecyloxy)-N-(2-hydroxyethyl)-N,N-dimethyl-6-oxohexan-1-aminium iodide:

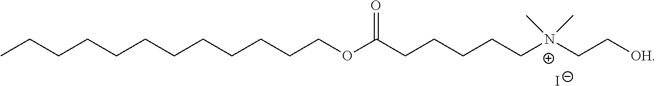 [SMILES: CCCCCCCCCCCCOC(=O)CCCCC[N+](C)(C)CCO ; [I-]]

Feature 25 is the compound of Feature 1, wherein the compound is 6-(dodecyloxy)-N-(2-ethoxy-2-oxoethyl)-N,N-dimethyl-6-oxohexan-1-aminium bromide:

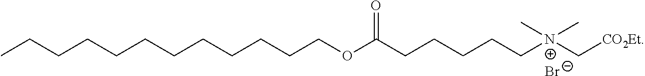 [SMILES: CCCCCCCCCCCCOC(=O)CCCCC[N+](C)(C)CC(=O)OCC ; [Br-]]

Feature 26 is the compound of Feature 1, wherein the compound is 6-(dodecyloxy)-N,N-dimethyl-6-oxo-N-(prop-2-yn-1-yl)hexan-1-aminium bromide:

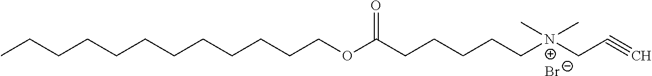 [SMILES: C#CC[N+](C)(C)CCCCCC(=O)OCCCCCCCCCCCC ; [Br-]]

Feature 27 is the compound of Feature 1, wherein the compound is 6-(dodecyloxy)-N-(3-hydroxypropyl)-N,N-dimethyl-6-oxohexan-1-aminium iodide:

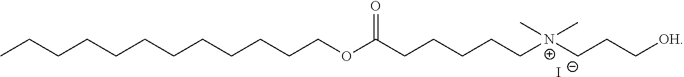

Feature 28 is the compound of Feature 1, wherein the compound is N-(2-(dimethylamino)-2-oxoethyl)-6-(dodecyloxy)-N,N-dimethyl-6-oxohexan-1-aminium iodide:

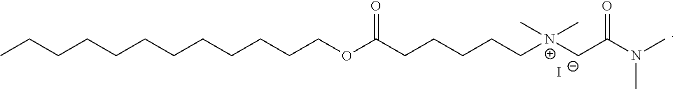 [SMILES: CCCCCCCCCCCCOC(=O)CCCCC[N+](C)(C)CC(=O)N(C)C ; [I-]]

Feature 29 is the compound of Feature 1, wherein the compound is N-benzyl-6-(dodecyloxy)-N,N-dimethyl-6-oxohexan-1-aminium bromide:

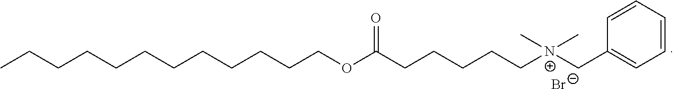 [SMILES: CCCCCCCCCCCCOC(=O)CCCCC[N+](C)(C)Cc1ccccc1 ; [Br-]]

Feature 30 is the compound of Feature 1, wherein the compound is 6-(dodecyloxy)-N-(2-(2-methoxyethoxy)ethyl)-N,N-dimethyl-6-oxohexan-1-aminium bromide:

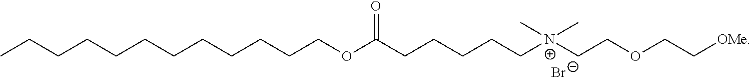 [SMILES: CCCCCCCCCCCCOC(=O)CCCCC[N+](C)(C)CCOCCOC ; [Br-]]

Feature 31 is the compound of Feature 1, wherein the compound is 6-(dodecyloxy)-N,N-dimethyl-6-oxo-N-phenethylhexan-1-aminium iodide:

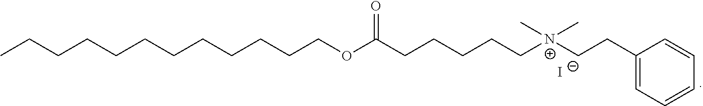

Feature 32 is the compound of Feature 1, wherein the compound is N-(2-(dimethylamino)-2-oxoethyl)-6-(dodecyloxy)-N-methyl-6-oxohexan-1-aminium chloride:

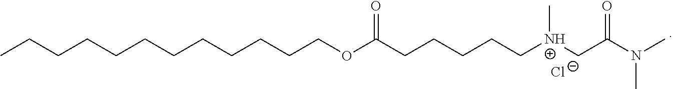 [SMILES: CCCCCCCCCCCCOC(=O)CCCCC[NH+](C)CC(=O)N(C)C ; [Cl-]]

Feature 33 is the compound of Feature 1, wherein the compound is sodium 4-((6-(dodecyloxy)-6-oxohexyl)(methyl)amino)butane-1-sulfonate:

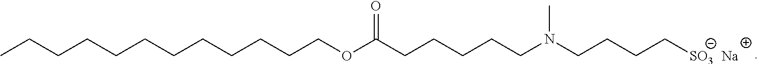

Feature 34 is the compound of Feature 1, wherein the compound is sodium 5-((6-(dodecyloxy)-6-oxohexyl)(methyl)amino)pentanoate:

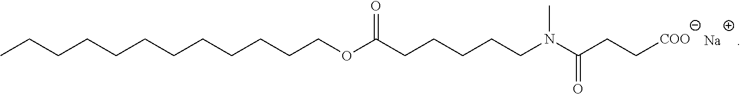

Feature 35 is the compound of Feature 1, wherein the compound is sodium 5-((6-(dodecyloxy)-6-oxohexyl)amino)pentanoate:

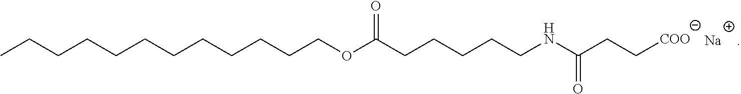

Feature 36 is the compound of Feature 1, wherein the compound is sodium 4-((6-(dodecyloxy)-6-oxohexyl)amino)butane-1-sulfonate:

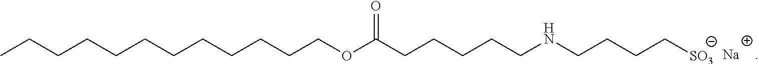

Feature 37 is the compound of Feature 1, wherein the compound is dodecyl 6-(2,3,4,5,6-pentahydroxy-N-methylhexanamido)hexanoate:

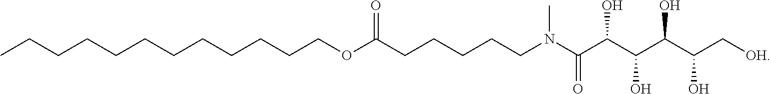 [SMILES: CCCCCCCCCCCCOC(=O)CCCCCN(C)C(=O)[C@H](O)[C@@H](O)[C@@H](O)[C@@H](O)CO]

Feature 38 is the surfactant of Formula (I) as defined in Feature 1.

Feature 39 is the use of a compound of Formula (I) as defined in Feature 1 as a surfactant.

Feature 40 is a compound of Formula II, wherein the compound of Formula II can be a salt, below:

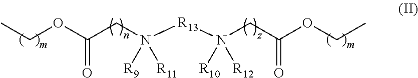

R₉, R₁₀, may be the same or different, and are selected from the group consisting of hydrogen and C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate, optionally, R₉ and R₁₀ are linked or otherwise connected together to form a 4 to 20 membered ring that may be substituted or unsubstituted, saturated or unsaturated; optionally the ring may include one or more of oxygen, nitrogen, or sulfur atoms or groups that include at least one of these atoms at any suitable position in the ring, and the ring may be optionally substituted with one or more substituents selected from the group consisting of any of C₁-C₆ alkyl, hydroxyl, amino, amido, sulfonyl, sulfonate, carbonyl, carboxyl, and carboxylate, wherein the ring formed from R₉ and R₁₀ is optionally linked and/or fused to a further ring;
R₁₁, R₁₂ are optionally present, and if present are the same or different, and are selected from the group consisting of hydrogen and one of C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate;
if R₁₁ and R₁₂ are present, a counter ion Z is selected from the group consisting of chloride, bromide, and iodide.
R₁₃ is selected from the group consisting of one of C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate;
Z is selected from the group consisting of chloride, bromide, and iodide.
n and z are, independently, an integer in the range of 2 to 5; and
m is an integer in the range of 9 to 20.

Feature 41 is compound of Feature 40, wherein R₉ and R₁₀ are linked or otherwise connected together to form a 4 to 20 membered ring that may be substituted or unsubstituted, saturated or unsaturated; optionally the ring may include one or more of oxygen, nitrogen, or sulfur atoms or groups that include at least one of these atoms at any suitable position on the ring, and the ring may be optionally substituted with one or more substituents selected from the group consisting of C₁-C₆ alkyl, hydroxyl, amino, amido, sulfonyl, sulfonate, carbonyl, carboxyl, and carboxylate, wherein the ring formed from R⁹ is optionally linked and/or fused to a further ring

Feature 42 is compound of Feature 40, wherein n is 5 and m is 11.

Feature 43 is compound of Feature 40, wherein Z is chloride or iodide.

Feature 44 is compound of Feature 40, wherein the compound is 1,4-bis(6-(dodecyloxy)-6-oxohexyl)piperazine-1,4-diium dichloride:

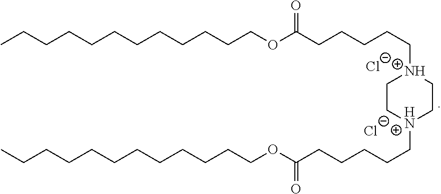

Feature 45 is compound of Feature 40, wherein the compound is didodecyl 6,6′-((2,3-dihydroxysuccinyl)bis(methylazanediyl))dihexanoate:

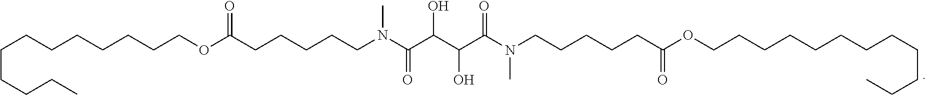 [SMILES: CCCCCCCCCCCCOC(=O)CCCCCN(C)C(=O)C(O)C(O)C(=O)N(C)CCCCCC(=O)OCCCCCCCCCCCC]

Feature 46 is compound of Feature 40, wherein the compound is 1,4-bis(6-(dodecyloxy)-6-oxohexyl)-1,4-dimethylpiperazine-1,4-diium diiodide:

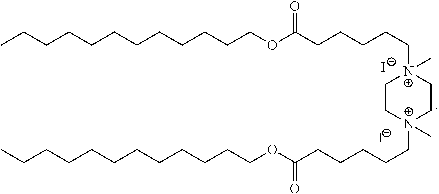 [SMILES: CCCCCCCCCCCCOC(=O)CCCCC[N+]1(C)CC[N+](C)(CCCCCC(=O)OCCCCCCCCCCCC)CC1 ; [I-] ; [I-]]

Feature 47 is a surfactant of Formula (II) as defined in Feature 40.

Feature 48 is the use of a compound of Formula (II) as defined in Feature 40 as a surfactant.

1. A compound of Formula I, wherein the compound of Formula I can be a salt, below: 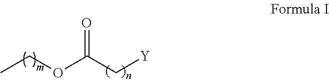
wherein Y is selected from formula (1-1), (1-2), or (1-3) 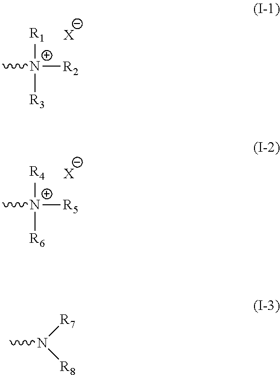
the wave-line denoting the connection to Formula I;
wherein
R₁ and R₂ form a ring that may be substituted or unsubstituted, saturated or unsaturated; optionally the ring may include one or more of oxygen, nitrogen, or sulfur atoms or groups that include at least one of these atoms at any suitable position on the ring, and the ring may be optionally substituted with one or more substituents selected from the group consisting of one of C₁-C₆ alkyl, hydroxyl, amino, amido, sulfonyl, sulfonate, carbonyl, carboxyl, and carboxylate;
wherein the ring formed from R₁ and R₂ is optionally linked and/or fused to a further ring;
R₃ is selected from the group consisting of hydrogen, one of C₁-C₆ alkyl, C₂-C₁₀ alkenyl, C₂-C₁₀ alkynyl, C₂-C₁₂ ester, hydroxyl, C₁-C₁₀ hydroxyl, aryl alkyl, C₂-C₁₂ alkoxy alkyl ether, alkyl phosphate, C₃-C₈ carboxylic acid, C₁-C₁₀ alkyl benzoic acid, oxygen, and sulfur;
R₄ and R₅ may be the same or different, and are selected from the group consisting of hydrogen and one of C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate;
R₆ is and is selected from the group consisting of C₂-C₁₀ alkenyl, C₂-C₁₀ alkynyl, C₂-C₁₂ ester, hydroxyl, aryl alkyl, C₂ C₁₂ alkoxy alkyl ether, alkyl phosphate, C₃-C₈ carboxylic acid, C₁-C₁₀ alkyl benzoic acid, amino, amido, and sulfur; wherein R⁶ may optionally be substituted with one or more substituents selected from the group consisting of aryl, heteroaryl, hydroxyl, amino, amido, sulfonyl, sulfonate, carbonyl, carboxyl, and carboxylate;
R₇ and R₈ may be the same or different, and are selected from the group consisting of hydrogen and one of C₁-C₆ alkyl, wherein the C₁-C₆ alkyl is substituted with one or more substituents selected from the group consisting of hydroxyl, amino, amido, sulfonyl, sulfonate, carbonyl, carboxyl, and carboxylate, or a salt thereof, wherein a counterion W associated with any of the hydroxyl, sulfonate, or carboxylate group is selected from Na, K or Ca;
X is selected from the group consisting of chloride, bromide, and iodide,
n is an integer in the range of 2 to 5; and
m is an integer in the range of 9 to 20.

2. The compound of claim 1, wherein Y is selected from piperidine, pyridine, pyrimidine, pyrrole, imidazole, pyrazole, piperazine, or morpholine.

3. The compound of claim 1, wherein Y is: 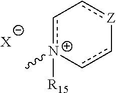
wherein:
R₁₅ is either not present, or an oxygen, hydrogen, one of C₁-C₆ alkyl, or one of C₁-C₆ heteroalkyl;
Z is 0, S, NR₁₆R₁₇, or CR₁₈R₁₉
n is an integer in the range of 2 to 5 and m is an integer in the range of 9 to 20;
X is selected from the group consisting of chloride, bromide, and iodide; and
R₁₆ and R₁₇ are each independently hydrogen or one of C₁-C₆ alkyl;
R₁₈ and R₁₉ are each independently either not present, or a hydrogen, or one of C₁-C₆ alkyl; [symbol]
are single bonds or double bonds; and
wherein the ring may be optionally substituted with one or more substituents selected from the group consisting of one of C₁-C₆ alkyl, hydroxyl, amino, amido, sulfonyl, sulfonate, carbonyl, carboxyl, and carboxylate.

4. The compound of claim 1, wherein R₃ and/or R₁₅ is oxygen.

5. The compound of claim 1, wherein R₃ and/or R₁₅ is C₁-C₆ alkyl or C₁-C₆ heteroalkyl.

6. The compound of claim 1, wherein R₃ and/or R₁₅ is C₁-C₆ alkyl substituted with hydroxy.

7. The compound of claim 3, wherein R₁₇ is methyl.

8. The compound of claim 3, wherein R₁₉ is hydrogen.

9. The compound of claim 1, wherein Y is: 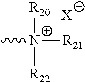
R₂₀ and R₂₁ are each independently methyl or a hydrogen;
n is an integer in the range of 2 to 5 and m is an integer in the range of 9 to 20;
X is selected from the group consisting of chloride, bromide, and iodide;
R₂₂ is selected from the group consisting of C₂-C₁₀alkenyl, C₂-C₁₀alkynyl, C₂-C₁₂ ester, hydroxyl, aryl C₁-C₆ alkyl, C₂-C₁₂ alkoxy alkyl ether, C₁-C₆ alkyl phosphate, C₃-C₈ carboxylic acid, C₁-C₁₀alkyl benzoic acid, amino, amido, and sulfur; wherein R₂₂ may optionally be substituted with one or more substituents selected from the group consisting of aryl, heteroaryl, hydroxyl, amino, amido, sulfonyl, sulfonate, carbonyl, carboxyl, and carboxylate.

10. The compound of claim 1, wherein Y is selected from:

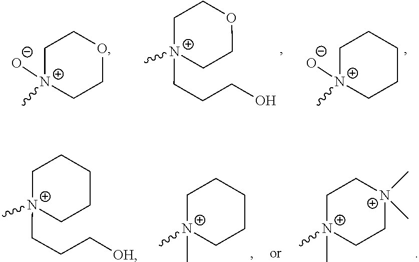

11. The compound of claim 1, wherein Y is selected from:

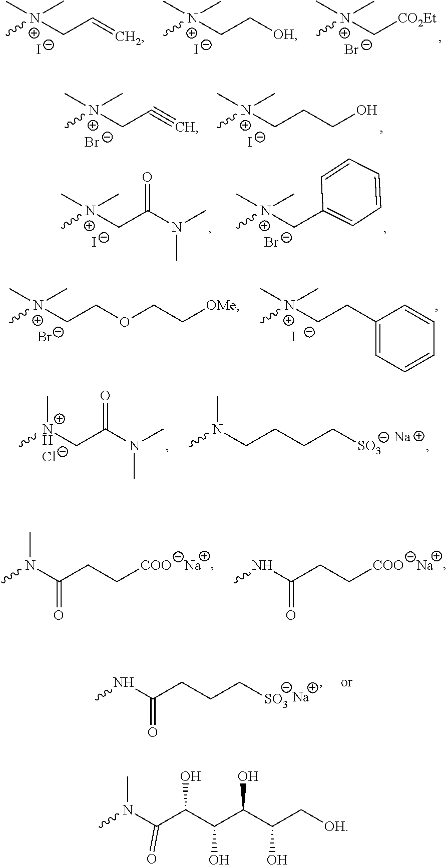

12. The compound of claim 1, wherein n is 3, 4, 5, 6, 7, 8, 9, 10, 11 or 12.

13. The compound of claim 1, wherein n is 5.

14. The compound of claim 1, wherein the counterion is present and is selected from the group consisting of chloride, bromide, and iodide.

15. The compound of claim 1, wherein the counterion is chloride.

16. The compound of claim 1, wherein the counterion is iodide.

17. The compound of claim 1, wherein the compound is selected from the group consisting of:
1-(6-(dodecyloxy)-6-oxohexyl)pyridin-1-ium bromide: 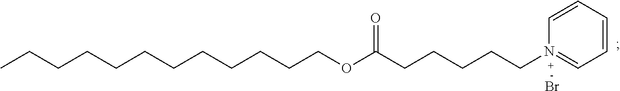
4-(6-(dodecyloxy)-6-oxohexyl)-4-(3-hydroxypropyl)morpholin-4-ium iodide: 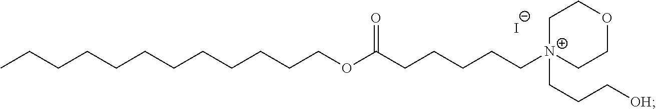 [SMILES: CCCCCCCCCCCCOC(=O)CCCCC[N+]1(CCCO)CCOCC1 ; [I-]]
1-(6-(dodecyloxy)-6-oxohexyl)piperidin-1-ium chloride: 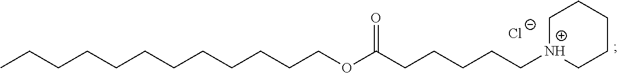 [SMILES: CCCCCCCCCCCCOC(=O)CCCCC[NH+]1CCCCC1 ; [Cl-]]
4-(6-(dodecyloxy)-6-oxohexyl)morpholin-4-ium chloride: 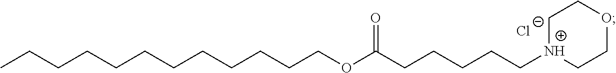 [SMILES: CCCCCCCCCCCCOC(=O)CCCCC[NH+]1CCOCC1 ; [Cl-]]
1-(6-(dodecyloxy)-6-oxohexyl)-4-methylpiperazine-1,4-diium dichloride: 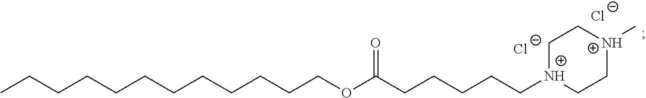 [SMILES: [Cl-] ; CCCCCCCCCCCCOC(=O)CCCCC[NH+]1CC[NH+](C)CC1 ; [Cl-]]
4-(6-(dodecyloxy)-6-oxohexyl)morpholine 4-oxide: 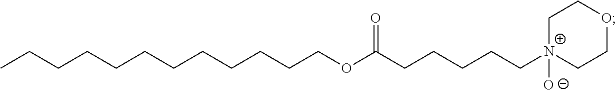 [SMILES: CCCCCCCCCCCCOC(=O)CCCCC[N+]1([O-])CCOCC1]
N-allyl-6-(dodecyloxy)-N,N-dimethyl-6-oxohexan-1-aminium iodide: 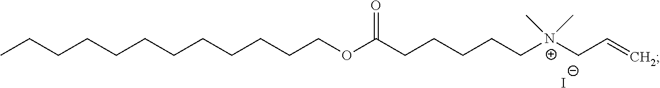 [SMILES: C=CC[N+](C)(C)CCCCCC(=O)OCCCCCCCCCCCC ; [I-]]
6-(dodecyloxy)-N-(2-hydroxyethyl)-N,N-dimethyl-6-oxohexan-1-aminium iodide: 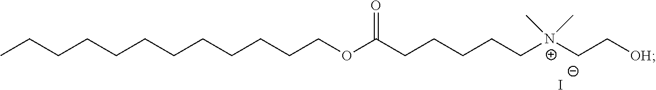 [SMILES: CCCCCCCCCCCCOC(=O)CCCCC[N+](C)(C)CCO ; [I-]]
6-(dodecyloxy)-N-(2-ethoxy-2-oxoethyl)-N,N-dimethyl-6-oxohexan-1-aminium bromide: 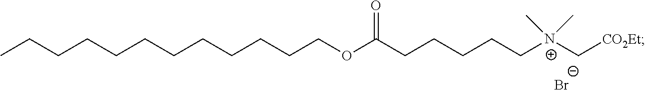 [SMILES: CCCCCCCCCCCCOC(=O)CCCCC[N+](C)(C)CC(=O)OCC ; [Br-]]
6-(dodecyloxy)-N,N-dimethyl-6-oxo-N-(prop-2-yn-1-yl)hexan-1-aminium bromide: 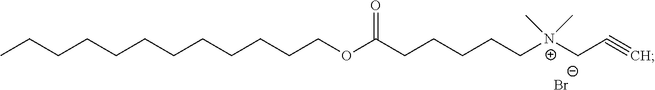 [SMILES: C#CC[N+](C)(C)CCCCCC(=O)OCCCCCCCCCCCC ; [Br-]]
6-(dodecyloxy)-N-(3-hydroxypropyl)-N,N-dimethyl-6-oxohexan-1-aminium iodide: 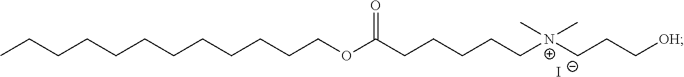 [SMILES: CCCCCCCCCCCCOC(=O)CCCCC[N+](C)(C)CCCO ; [I-]]
N-(2-(dimethylamino)-2-oxoethyl)-6-(dodecyloxy)-N,N-dimethyl-6-oxohexan-1-aminium iodide: 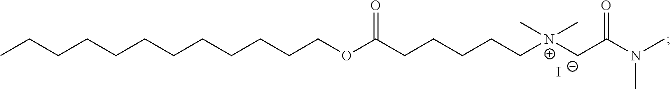 [SMILES: CCCCCCCCCCCCOC(=O)CCCCC[N+](C)(C)CC(=O)N(C)C ; [I-]]
N-benzyl-6-(dodecyloxy)-N,N-dimethyl-6-oxohexan-1-aminium bromide: 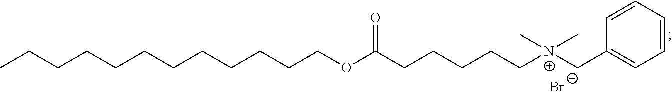 [SMILES: CCCCCCCCCCCCOC(=O)CCCCC[N+](C)(C)Cc1ccccc1 ; [Br-]]
6-(dodecyloxy)-N-(2-(2-methoxyethoxy)ethyl)-N,N-dimethyl-6-oxohexan-1-aminium bromide: 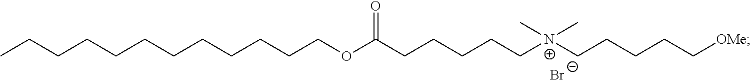 [SMILES: CCCCCCCCCCCCOC(=O)CCCCC[N+](C)(C)CCCCCOC ; [Br-]]
6-(dodecyloxy)-N,N-dimethyl-6-oxo-N-phenethylhexan-1-aminium iodide: 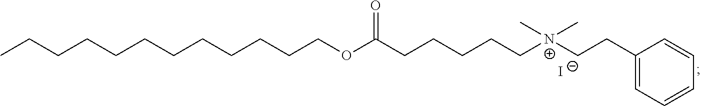 [SMILES: CCCCCCCCCCCCOC(=O)CCCCC[N+](C)(C)CCc1ccccc1 ; [I-]]
N-(2-(dimethylamino)-2-oxoethyl)-6-(dodecyloxy)-N-methyl-6-oxohexan-1-aminium chloride: 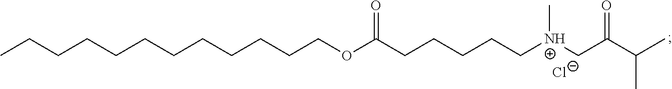 [SMILES: CCCCCCCCCCCCOC(=O)CCCCC[NH+](C)CC(=O)C(C)C ; [Cl-]]
sodium 4-((6-(dodecyloxy)-6-oxohexyl)(methyl)amino)butane-1-sulfonate: 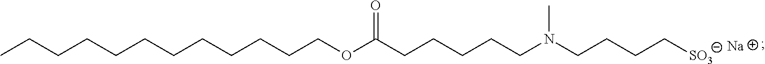
sodium 5-((6-(dodecyloxy)-6-oxohexyl)(methyl)amino)pentanoate: 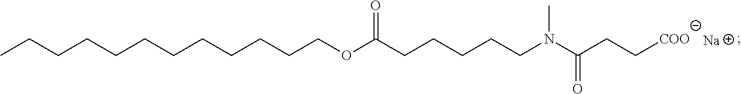
sodium 5-((6-(dodecyloxy)-6-oxohexyl)amino)pentanoate: 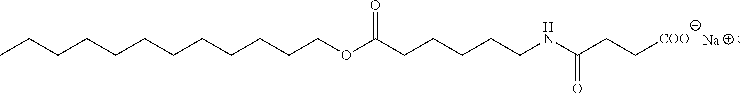
sodium 4-((6-(dodecyloxy)-6-oxohexyl)amino)butane-1-sulfonate: 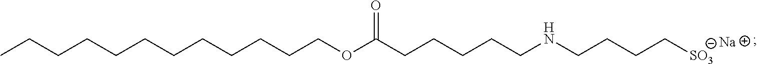
dodecyl 6-(2,3,4,5,6-pentahydroxy-N-methylhexanamido)hexanoate; and 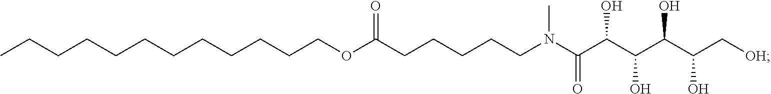 [SMILES: CCCCCCCCCCCCOC(=O)CCCCCN(C)C(=O)[C@H](O)[C@@H](O)[C@@H](O)[C@@H](O)CO]
combinations of the foregoing.

18. A compound of Formula II, wherein the compound of Formula II can be a salt, below: 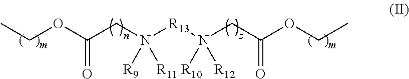
R₉, R₁₀, may be the same or different, and are selected from the group consisting of hydrogen and C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate, optionally, R₉ and R₁₀ are linked or otherwise connected together to form a 4 to 20 membered ring that may be substituted or unsubstituted, saturated or unsaturated; optionally the ring may include one or more of oxygen, nitrogen, or sulfur atoms or groups that include at least one of these atoms at any suitable position on the ring, and the ring may be optionally substituted with one or more substituents selected from the group consisting of any one of C₁-C₆alkyl, hydroxyl, amino, amido, sulfonyl, sulfonate, carbonyl, carboxyl, and carboxylate, wherein the ring formed from R⁹ is optionally linked and/or fused to a further ring;
R₁₁, R₁₂ are optionally present, and if present are the same or different, and are selected from the group consisting of hydrogen and C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate;
if R₁₁ and R₁₂ are present, a counter ion Z is selected from the group consisting of chloride, bromide, and iodide.
R₁₃ is selected from the group consisting of one of C₁-C₆ alkyl, wherein the C₁-C₆ alkyl may optionally be substituted with one or more substituents selected from the group consisting of hydroxyl, amino, amido, sulfonyl, sulfonate, carbonyl, carboxyl, and carboxylate;
Z is selected from the group consisting of chloride, bromide, and iodide.
n and z are, independently, an integer fin the range of 2 to 5; and
m is an integer in the range of 9 to 20.

19. The compound of claim 18, wherein R₉ and R₁₀ are linked or otherwise connected together to form a 4 to 20 membered ring that may be substituted or unsubstituted, saturated or unsaturated; optionally the ring may include one or more of oxygen, nitrogen, or sulfur atoms or groups that include at least one of these atoms at any suitable position on the ring, and the ring may be optionally substituted with one or more substituents selected from the group consisting of any one of C₁-C₆ alkyl, hydroxyl, amino, amido, sulfonyl, sulfonate, carbonyl, carboxyl, and carboxylate, wherein the ring formed from R⁹ is optionally linked and/or fused to a further ring

20. The compound of claim 18, wherein n is 5 and m is 11.

21. The compound of claim 18, wherein Z is chloride or iodide.

22. The compound of claim 18, wherein the compound is selected from:
1,4-bis(6-(dodecyloxy)-6-oxohexyl)piperazine-1,4-diium dichloride: 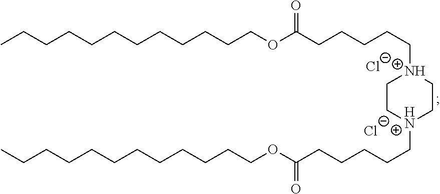
didodecyl 6,6′-((2,3-dihydroxysuccinyl)bis(methylazanediyl))dihexanoate: 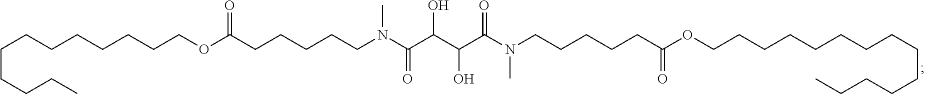 [SMILES: CCCCCCCCCCCCCCOC(=O)CCCCCN(C)C(=O)C(O)C(O)C(=O)N(C)CCCCCC(=O)OCCCCCCCCCCCC]
and 1,4-bis(6-(dodecyloxy)-6-oxohexyl)-1,4-dimethylpiperazine-1,4-diium diiodide: 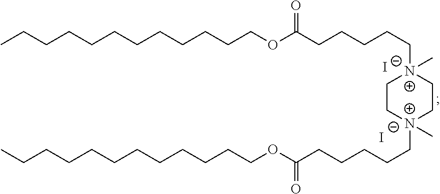 [SMILES: CCCCCCCCCCCCOC(=O)CCCCC[N+]1(C)CC[N+](C)(CCCCCC(=O)OCCCCCCCCCCCC)CC1 ; [I-] ; [I-]]
and combinations of the foregoing.
